(19) United States
(12) Patent Application Publication
(10) Pub. No.: US 2010/0222218 A1
(43) Pub. Date: Sep. 2, 2010

(54) INSECTICIDAL HETEROCYCLIC CARBOXYLIC ACID DERIVATIVES

(75) Inventors: Reiner Fischer, Monheim (DE); Herbert Gayer, Monheim (DE); Olaf Gebauer, Leverkusen (DE); Oliver Gaertzen, Koln (DE); Ulrich Heinemann, Leichlingen (DE); Stefan Herrmann, Langenfeld (DE); Michael Müller, Leverkusen (DE); Thomas Schenke, Bergisch Gladbach (DE); Welf-Burkhard Wiese, Langenfeld (DE); Heinz-Juergen Wroblowsky, Langenfeld (DE); Eva-Maria Franken, Leichlingen (DE); Olga Malsam, Rosrath (DE); Ulrich Görgens, Ratingen (DE); Christian Arnold, Langenfeld (DE)

Correspondence Address: Baker Donelson Bearman, Caldwell & Berkowitz, PC, 555 Eleventh Street, NW, Sixth Floor, Washington, DC, 20004, US

(73) Assignee: Bayer CropScience AG, Monheim (DE)

(21) Appl. No.: 12/377,566

(22) PCT Filed: Jun. 14, 2007
(86) PCT No.: PCT/EP07/05251

§ 371 Date: Apr. 23, 2009

(30) Foreign Application Priority Data
Aug. 17, 2006 (DE) 10 2006 038 617.5
Aug. 22, 2006 (DE) 10 2006 039 255.8

(51) Int. Cl. A01N 25/26; C07D 487/04; A01N 43/90; C07D 413/14; A01N 43/84; A01P 5/00; A01P 7/02; A01P 7/04; A01P 17/00

(52) U.S. Cl. 504/100; 544/281; 514/259.3; 544/117; 514/233.2; 504/241; 504/225

(57) ABSTRACT

The invention relates to the use of heterocyclic carboxylic acid derivatives of the formula (I) below

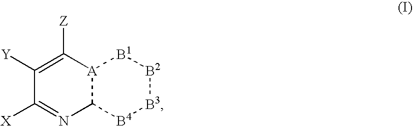

in which the symbols have the meanings given in the description, or their agrochemically active salts or of mixtures of these compounds and/or their agrochemically active salts with other active compounds for controlling animal pests, to methods and compositions for controlling animal pests in and/or on plants or in and/or on seed of plants, to processes for preparing such compositions and treated seed.

The present invention relates to the use of heterocyclic carboxylic acid derivatives of the formula (I) below

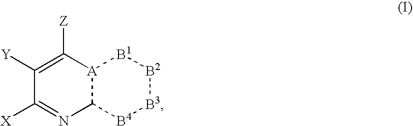

in which the symbols have the meanings given in the description, or to their agrochemically active salts or to mixtures of these compounds and/or their agrochemically active salts with other active compounds for controlling animal pests and/or phytopathogenic harmful fungi, to methods and compositions for controlling animal pests and/or phytopathogenic harmful fungi in and/or on plants or in and/or on seed of plants, to processes for preparing such compositions and to treated seed.

The present invention furthermore relates to heterocyclic carboxylic acid derivatives of the formula (I), to processes for their preparation and to their use for controlling pests in agriculture, horticulture and forestry, in the protection of materials and also in the domestic and hygiene field.

The present invention also relates to enhancing the activity of crop protection compositions comprising insecticidal heterocyclic carboxylic acid derivatives of the formula (I) by addition of ammonium or phosphonium salts and, if appropriate, penetrants, to the corresponding compositions, to processes for their preparation and to their use in crop protection, in particular as insecticides and/or acaricides.

It is already known that certain pyrazolopyrimidine derivatives have fungicidal properties, see, for example, WO 04/000844, WO 05/082907 and WO 06/087120. Furthermore, it is already known that certain pyrazolopyrimidine derivatives have insecticidal properties, see, for example, WO 04/000844.

However, since the environmental and economic requirements imposed on modern-day pesticides are continually increasing, with regard, for example, to the spectrum of action, toxicity, selectivity, application rate, formation of residues, and favourable preparability, and since, furthermore, there may be problems, for example, with resistances, a constant task is to develop novel pesticides which in some areas at least have advantages over their known counterparts.

This invention now provides novel heterocyclic carboxylic acid derivatives of the formula (I)

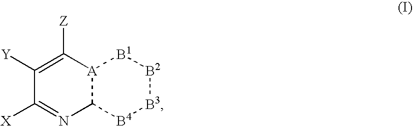

in which the symbols have the following meaning:
A represents a nitrogen atom, in which case the bond in the pyrimidine ring is a single bond, or represents a carbon atom, in which case the bond in the pyridine ring is a double bond,
B¹ represents a nitrogen atom —N═ or represents the fragment

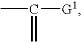

B² represents a nitrogen atom —N═ or represents the fragment

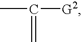

B³ represents a nitrogen atom —N═ or represents the fragment

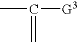

or represents a bond,
B⁴ represents the fragment

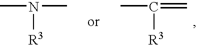

where the dashed bonds may be single bonds, double bonds or aromatic bonds;
X represents hydrogen, halogen, cyano, hydroxyl, optionally substituted alkyl, optionally substituted alkoxy, optionally substituted phenyl, optionally substituted alkylthio, optionally substituted alkylsulphinyl or optionally substituted alkylsulphonyl;
Y represents optionally substituted aryl, optionally substituted heterocyclyl, optionally substituted alkyl, optionally substituted alkenyl, optionally substituted alkynyl, optionally substituted cycloalkyl, optionally substituted cycloalkenyl, optionally substituted arylalkyl, halogen, an optionally substituted amino group, optionally substituted (C₁-C₈)-alkoxy, optionally substituted (C₁-C₈)-alkylthio, optionally substituted (C₆-C₁₀)-aryloxy, optionally substituted (C₆-C₁₀)-arylthio, optionally substituted heterocyclyloxy, optionally substituted (C₆-C₁₀)-aryl-(C₁-C₄)-alkoxy, optionally substituted (C₆-C₁₀)-aryl-(C₁-C₄)-alkylthio, optionally substituted heterocyclyl-(C₁-C₄)-alkoxy, optionally substituted heterocyclyl-(C₁-C₄)-alkylthio, C(S)OR⁸, C(O)SR⁸ or C(S)SR⁸;
Z represents hydroxyl, halogen or in each case optionally substituted alkoxy, alkylthio, alkylsulphonyl or the group

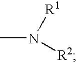

G¹, G² and G³ independently of one another represent hydrogen, halogen, optionally substituted alkyl or optionally substituted cycloalkyl, O—(C₁-C₄)-alkyl or S(O)₀₋₂(C₁-C₄)-alkyl;
L represents oxygen or sulphur;
R¹ represents hydrogen, optionally substituted alkyl, optionally substituted alkenyl, optionally substituted alkynyl, optionally substituted cycloalkyl or optionally substituted heterocyclyl, hydroxyl, optionally substituted alkoxy, amine, optionally substituted alkylamine or optionally substituted dialkylamine;
R² represents hydrogen or alkyl;
or
R¹ and R² together with the nitrogen atom to which they are attached represent an optionally substituted heterocyclic ring;
R³ represents

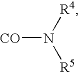

### CO—OR⁶, CO—SR⁶, CS—OR⁶ or CS—SR⁶;

R⁴ represents hydrogen, in each case optionally substituted alkyl, alkenyl or alkynyl, in each case optionally substituted saturated or unsaturated cycloalkyl which may optionally be interrupted by heteroatoms, in each case optionally substituted phenyl, heteroaryl, arylalkyl, heteroarylalkyl or a cation such as, for example, a mono- or divalent metal atom or an optionally alkyl- or arylalkyl-substituted ammonium ion;
R⁵ represents hydrogen, in each case optionally substituted alkyl, alkoxy, alkenyl, alkenyloxy or alkynyl, represents the groups COR⁷, S(O)₁₋₂R⁷, cyano, COOR⁷,

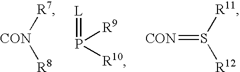

saturated, partially or fully unsaturated or aromatic, optionally substituted 5- or 6-membered heterocyclyl which optionally contains one or up to three further heteroatoms selected from the group consisting of nitrogen, sulphur and oxygen atoms, where oxygen atoms must not be adjacent to one another; or
R⁴ and R⁵ together with the nitrogen atom to which they are attached represent an optionally substituted, saturated, unsaturated or aromatic ring which may optionally be interrupted by further heteroatoms;
R⁶ represents hydrogen, a cation, for example an optionally alkyl- or arylalkyl-substituted ammonium ion, in each case optionally substituted alkyl, alkenyl, alkynyl, in each case optionally substituted cycloalkyl or cycloalkenyl which may optionally be interrupted by a heteroatom, optionally substituted cycloalkylalkyl or optionally substituted arylalkyl;
R⁷ represents hydrogen, in each case optionally substituted alkyl, alkenyl, alkynyl, represents in each case optionally substituted cycloalkylalkyl, cycloalkyl or cycloalkenyl, in each case optionally substituted aryl, heteroaryl, arylalkyl or heteroarylalkyl;
R⁸ represents hydrogen, in each case optionally substituted alkyl, alkenyl, alkynyl, alkoxy or NH—R⁴;
or
R⁴ and R⁷ together with the N—CO or N—S(O)₁₋₂ group to which they are attached form a 4- to 8-membered cycle which may contain one or more heteroatoms from the group consisting of sulphur, oxygen and nitrogen, where oxygen atoms must not be adjacent to one another;
or
R⁷ and R⁸ together with the nitrogen atom to which they are attached represent an optionally substituted saturated or unsaturated ring which may optionally be interrupted by further heteroatoms;
R⁹ and R¹⁰ independently of one another represent in each case optionally substituted alkyl, alkenyl, alkoxy, alkenyloxy, alkynyloxy, cycloalkoxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy or heteroarylalkylthio;
or
R⁹ and R¹⁰ together with the phosphorus atom to which they are attached represent an optionally substituted 5- to 7-membered cycle which may be interrupted by one or two oxygen and/or sulphur atoms; and
R¹¹ and R¹² independently of one another represent in each case optionally substituted alkyl, alkenyl, alkynyl, phenyl or phenylalkyl;
   and agrochemically active salts thereof. The radicals referred to above as optionally substituted, including groups and rings or cycles, can be unsubstituted or substituted, in particular by the same substituents as defined for the corresponding radicals in the further embodiments of the present invention described below. Here, the substituents of one embodiment may be combined as desired with those of another embodiment.

Heterocyclic carboxylic acid derivatives of the formula (I) according to the invention and their agrochemically active salts are highly suitable for use as pesticides, in particular for controlling animal pests, such as insects, parasites of the sub-class of the Acari (Acarina) (such as mites, spider mites and/or ticks) and/or nematodes. The compounds according to the invention mentioned above show in particular strong insecticidal and/or acaricidal and/or nematicidal activity and can be used both in crop protection, in the domestic and hygiene field and in the protection of materials.

The compounds of the formula (I) may be present either in pure form or as mixtures of various possible isomeric forms, in particular of stereoisomers,such as E and Z, threo and erythro, and also optical isomers, such as R and S isomers or atropisomers, and, if appropriate, also of tautomers. The invention comprises both the pure isomers and mixtures thereof.

If appropriate, the compounds of the formula (I) may be present in various polymorphic forms or as mixtures of differing polymorphic forms. Both the pure polymorphs and the polymorph mixtures are provided by the invention and can be used according to the invention.

Depending on the nature of the substituents defined above, the compounds of the formula (I) have acidic or basic properties and may form salts, if appropriate also inner salts. If the compounds of the formula (I) carry hydroxyl groups, carboxyl groups or other groups which induce acidic properties, these compounds can be converted with bases into salts. Suitable bases are, for example, hydroxides, carbonates, bicarbonates of the alkali metals and alkaline earth metals, in particular those of sodium, potassium, magnesium and calcium, furthermore ammonia, primary, secondary and tertiary amines having (C₁-C₄)-alkyl radicals, mono-, di- and trialkanolamines of (C₁-C₄)-alkanols, choline and also chlorocholine. If the compounds of the formula (I) carry amino groups, alkylamino groups or other groups which induce basic properties, these compounds can be converted with acids into salts. Suitable acids are, for example, mineral acids, such as hydrochloric acid, sulphuric acid and phosphoric acid, organic acids, such as acetic acid or oxalic acid, and acidic salts, such as NaHSO₄ and KHSO₄. The salts obtainable in this manner also have insecticidal properties.

The formula (I) provides a general definition of the compounds according to the invention, i.e. the heterocyclic carboxylic acid derivatives.

Accordingly, the present invention relates to
(I) the use of chemical compounds, i.e. heterocyclic carboxylic acid derivatives of the formula (I) below

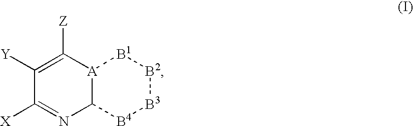

in which
A represents a nitrogen atom, in which case the bond in the pyrimidine ring is a single bond, or represents a carbon atom, in which case the bond in the pyridine ring is a double bond,
B¹ represents a nitrogen atom —N═ or represents the fragment

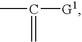

B² represents a nitrogen atom —N═ or represents the fragment

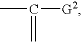

B³ represents a nitrogen atom —N═ or represents the fragment

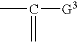

or represents a bond,
and
B⁴ represents the fragment

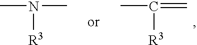

where the dashed bonds may be single bonds, double bonds or aromatic bonds;
X represents hydrogen, halogen, cyano, hydroxyl, alkyl, alkoxy, phenyl, alkylthio, alkylsulphinyl or alkylsulphonyl;
Y represents aryl, heterocyclyl, alkyl, alkenyl, alkynyl, cycloalkyl, cycloalkenyl, arylalkyl, halogen, an amino group, (C₁-C₈)-alkoxy, (C₁-C₈)-alkylthio, (C₆-C₁₀)-aryloxy, (C₆-C₁₀)-arylthio, heterocyclyloxy, (C₆-C₁₀)-aryl-(C₁-C₄)-alkoxy, (C₆-C₁₀)-aryl-(C₁-C₄)-alkylthio, heterocyclyl-(C₁-C₄)-alkoxy, heterocyclyl-(C₁-C₄)-alkylthio, C(S)OR⁸, C(O)SR⁸ or C(S)SR⁸;
Z represents hydroxyl, halogen or alkoxy, alkylthio, alkylsulphonyl or the group

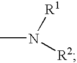

G¹, G² and G³ independently of one another represent hydrogen, halogen, alkyl or cycloalkyl, O—(C₁-C₄)-alkyl or S(O)₀₋₂(C₁-C₄)-alkyl;
L represents oxygen or sulphur;
R¹ represents hydrogen, alkyl, alkenyl, alkynyl, cycloalkyl or heterocyclyl, hydroxyl, alkoxy, amine, alkylamine or dialkylamine;
R² represents hydrogen or alkyl;
or
R¹ and R² together with the nitrogen atom to which they are attached represent a heterocyclic ring;
R³ represents

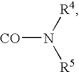

### CO—OR⁶, CO—SR⁶, CS—OR⁶ or CS—SR⁶;

R⁴ represents hydrogen, alkyl, alkenyl or alkynyl, in each case saturated or unsaturated cycloalkyl which may optionally be interrupted by heteroatoms, phenyl, heteroaryl, arylalkyl or heteroarylalkyl or a cation such as, for example, a mono- or divalent metal atom or an ammonium ion;
R⁵ represents hydrogen, alkyl, alkoxy, alkenyl, alkenyloxy or alkynyl, represents the groups COR⁷, S(O)₁₋₂R⁷, cyano, COOR⁷,

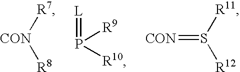

or saturated, partially or fully unsaturated or aromatic, 5- or 6-membered heterocyclyl which optionally contains one or up to three further heteroatoms selected from the group consisting of nitrogen, sulphur and oxygen atoms, where oxygen atoms must not be adjacent to one another;
or
R⁴ and R⁵ together with the nitrogen atom to which they are attached represent a saturated, unsaturated or aromatic ring which may optionally be interrupted by further heteroatoms;
R⁶ represents hydrogen, a cation, for example an ammonium ion, alkyl, alkenyl, alkynyl, cycloalkyl or cycloalkenyl which may optionally be interrupted by a heteroatom, cycloalkylalkyl or aryl, arylalkyl, heteroaryl or heteroarylalkyl;
R⁷ represents hydrogen, alkyl, alkenyl, alkynyl, represents cycloalkylalkyl, cycloalkyl or cycloalkenyl, aryl, heteroaryl, arylalkyl or heteroarylalkyl;
R⁸ represents hydrogen, alkyl, alkenyl, alkynyl, alkoxy or NH—R⁴;
or
R⁴ and R⁷ together with the N—CO or N—S(O)₁₋₂ group to which they are attached form a 4- to 8-membered cycle which may contain one or more heteroatoms from the group consisting of sulphur, oxygen and nitrogen, where oxygen atoms must not be adjacent to one another;
or
R⁷ and R⁸ together with the nitrogen atom to which they are attached represent an optionally substituted saturated or unsaturated ring which may optionally be interrupted by further heteroatoms;
R⁹ and R¹⁰ independently of one another represent alkyl, alkenyl, alkoxy, alkenyloxy, alkynyloxy, cycloalkoxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy or heteroarylalkylthio;
or
R⁹ and R¹⁰ together with the phosphorus atom to which they are attached represent an optionally substituted 5- to 7-membered cycle which may be interrupted by one or two oxygen and/or sulphur atoms;
and
R¹¹ and R¹² independently of one another represent alkyl, alkenyl, alkynyl, phenyl or phenylalkyl;
   or agrochemically active salts thereof for controlling animal pests;
(2) the use of a mixture comprising at least one of the compounds defined under (1) and/or at least one of the agrochemically active salts thereof and a further active compound selected from the group consisting of insecticides, attractants, sterilants, bactericides, acaricides, nematicides, fungicides, growth regulators, herbicides, safeners, fertilizers and semiochemicals for controlling animal pests;
(3) a composition for controlling animal pests in and/or on plants or in and/or on seed of plants, where the composition mentioned comprises at least one of the compounds defined under (1) and/or at least one of the agrochemically active salts thereof or a mixture as defined under (2) and agrochemically customary auxiliaries and/or additives;
(4) a method for controlling animal pests in and/or on plants or in and/or on seed of plants which comprises bringing the pests mentioned into direct or indirect contact with at least one of the compounds defined under (1) and/or at least one of the agrochemically active salts thereof or a mixture as defined under (2) or a composition as defined under (3);
(5) a process for preparing a composition as defined under (3) which comprises mixing at least one of the compounds defined under (1) and/or at least one of the agrochemically active salts thereof or a mixture as defined under (2) with agrochemically customary auxiliaries and/or additives; and
(6) seed treated with at least one of the compounds as defined under (1) and/or at least one of the agrochemically active salts thereof or a mixture as defined under (2) or a composition as defined under (3).

Hereinbelow, preferred substituents, fragments or ranges of the radicals listed in the formulae mentioned above and below are illustrated for a preferred embodiment of the present invention:
A preferably represents a nitrogen atom, in which case the bond in the pyrimidine ring is a single bond, or represents a carbon atom, in which case the bond in the pyridine ring is a double bond,
B¹ preferably represents a nitrogen atom —N═ or represents the fragment

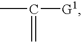

B² preferably represents a nitrogen atom —N═ or represents the fragment

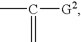

B³ preferably represents a nitrogen atom —N═ or represents the fragment

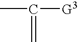

or represents a bond,
B⁴ preferably represents the fragment

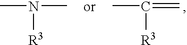

where the dashed bonds may be single bonds, double bonds or aromatic bonds;
X preferably represents hydrogen, fluorine, chlorine, bromine, cyano, hydroxyl, alkoxy having 1 to 4 carbon atoms or alkylthio having 1 to 4 carbon atoms, C₁-C₆-alkyl or C₁-C₄-haloalkyl;
Y preferably represents C₁-C₁₀-alkyl, C₂-C₁₀-alkenyl, C₂-C₁₀-alkynyl, C₃-C₈-cycloalkyl, phenyl-C₁-C₁₀-alkyl, where Y is unsubstituted or partially or fully halogenated and/or optionally carries one to three radicals R^{x}, or C₁-C₁₀-haloalkyl, which optionally carries one to three radicals R^{x}, where the R^{x} are identical or different and selected from the group consisting of cyano, nitro, hydroxyl, C₁-C₆-alkyl, C₁-C₆-haloalkyl, C₃-C₆-cycloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylamino, di-C₁-C₆-alkylamino, C₂-C₆-alkenyl, C₂-C₆-alkenyloxy, C₂-C₆-alkynyl, C₃-C₆-alkynyloxy and optionally halogenated oxy-C₁-C₄-alkyl-C₁-C₄-alkenoxy, oxy-C₁-C₄-alkenyl-C₁-C₄-alkoxy and oxy-C₁-C₄-alkyl-C₁-C₄-alkyloxy; or
Y preferably represents phenyl which may optionally be mono- to tetrasubstituted by identical or different substituents selected from the group consisting of

halogen, cyano, nitro, amino, hydroxyl, formyl, carboxy, carboxyalkyl, carbamoyl, thiocarbamoyl;

in each case straight-chain or branched alkyl, alkoxy, alkylthio, alkylsulphinyl or alkylsulphonyl having in each case 1 to 6 carbon atoms;

in each case straight-chain or branched alkenyl, alkynyl, alkynyloxy or alkenyloxy having in each case 2 to 6 carbon atoms;

in each case straight-chain or branched haloalkyl, haloalkoxy, haloalkylthio, haloalkylsulphinyl or haloalkylsulphonyl having in each case 1 to 6 carbon atoms and 1 to 13 identical or different halogen atoms;

in each case straight-chain or branched haloalkenyl or haloalkenyloxy having in each case 2 to 6 carbon atoms and 1 to 11 identical or different halogen atoms;

in each case straight-chain or branched alkylamino, dialkylamino, alkylcarbonyl, alkylcarbonyloxy, alkoxycarbonyl, alkylsulphonyloxy, hydroximinoalkyl or alkoximinoalkyl having in each case 1 to 6 carbon atoms in the individual alkyl moieties;

cycloalkyl having 3 to 8 carbon atoms which is optionally mono- or disubstituted by fluorine, chlorine, C₁-C₃-alkyl or C₁-C₃-alkoxy;

2,3- or 3,4-attached 1,3-propanediyl, 1,4-butanediyl, methylenedioxy (—O—CH₂—O—) or 1,2-ethylenedioxy (—O—CH₂—CH₂—O—), where these radicals may be mono- or polysubstituted by identical or different substituents from the group consisting of halogen, alkyl having 1 to 4 carbon atoms and haloalkyl having 1 to 4 carbon atoms and 1 to 9 identical or different halogen atoms; or
Y preferably represents saturated or fully or partially unsaturated or aromatic heterocyclyl having 3 to 8 ring members and 1 to 3 heteroatoms from the group consisting of nitrogen, oxygen and sulphur, where the heterocyclyl may be mono- or disubstituted by halogen, alkyl having 1 to 4 carbon atoms, alkoxy having 1 to 4 carbon atoms, alkylthio having 1 to 4 carbon atoms, haloalkoxy having 1 to 4 carbon atoms, haloalkylthio having 1 to 4 carbon atoms, hydroxyl, mercapto, cyano, nitro and/or cycloalkyl having 3 to 6 carbon atoms or/and carboxyalkyl;
Z preferably represents hydroxyl, chlorine, bromine, straight-chain or branched C₁-C₆-alkoxy, C₁-C₆-alkylthio or C₁-C₆-alkylsulphonyl, each of which is optionally substituted by 1 to 7 halogen atoms, or the group

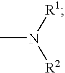

G¹, G² and G³ preferably independently of one another represent hydrogen, halogen, C₁-C₄-alkyl which is optionally substituted by one or more halogen atoms, C₃-C₆-cycloalkyl which is optionally substituted by one or more halogen atoms, SCH₃, SC₂H₅, SOCH₃, SOC₂H₅, SO₂CH₃, SO₂C₂H₅, OCH₃ or OC₂H₅;
L represents oxygen or sulphur;
R¹ preferably represents hydrogen, alkyl having 1 to 10 carbon atoms which is unsubstituted or mono- to pentasubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms; or
R¹ preferably represents alkenyl having 3 to 10 carbon atoms which is unsubstituted or mono- to trisubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms; or
R¹ preferably represents alkynyl having 3 to 10 carbon atoms which is unsubstituted or mono- to trisubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms; or
R¹ preferably represents cycloalkyl having 3 to 10 carbon atoms which is unsubstituted or mono- to trisubstituted by identical or different substituents from the group consisting of halogen and alkyl, haloalkyl having in each case 1 to 4 carbon atoms; or
R¹ preferably represents saturated or unsaturated heterocyclyl having 3 to 10 ring members and 1 to 3 heteroatoms, such as nitrogen, oxygen and/or sulphur, where the heterocyclyl is unsubstituted or mono- or polysubstituted by halogen, alkyl having 1 to 4 carbon atoms, alkoxy having 1 to 4 carbon atoms, cyano, nitro, cycloalkyl having 3 to 6 carbon atoms, hydroxyl, alkoxy, alkenyloxy, alkynyloxy having 1 to 6 carbon atoms or mercapto;
R² preferably represents hydrogen or alkyl having 1 to 6 carbon atoms; or
R¹ and R² together with the nitrogen atom to which they are attached preferably represent a saturated or unsaturated heterocyclic ring having 3 to 8 ring members, where the heterocycle optionally contains a further nitrogen, oxygen or sulphur atom as ring member and where the heterocycle may be unsubstituted or up to trisubstituted by fluorine, chlorine, bromine, alkyl having 1 to 4 carbon atoms, haloalkyl having 1 to 4 carbon atoms and 1 to 9 fluorine and/or chlorine atoms, hydroxyl, alkoxy having 1 to 4 carbon atoms, haloalkoxy having 1 to 4 carbon atoms and 1 to 9 fluorine and/or chlorine atoms, mercapto, thioalkyl having 1 to 4 carbon atoms and/or haloalkylthio having 1 to 4 carbon atoms and 1 to 9 fluorine and/or chlorine atoms;
R³ preferably represents

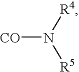

### CO—OR⁶, CO—SR⁶, CS—OR⁶ or CS—SR⁶;

R⁴ preferably represents hydrogen, cations from the group of the alkali metals or alkaline earth metals or ammonium ions NH₄, mono-(C₁-C₁₀)-alkylammonium, di-(C₁-C₁₀)-alkyl-ammonium, tri-(C₁-C₁₀)-alkylammonium, tetra-(C₁-C₁₀)-alkylammonium, where the alkyl radicals of the ammonium ions may be substituted by aryl or hydroxyl, or cholinium,
   represents C₁-C₁₀-alkyl, C₃-C₁₀-alkenyl or C₃-C₁₀-alkynyl, each of which is optionally mono- or polysubstituted by fluorine, chlorine, C₁-C₄-alkoxy, C₁-C₄-alkylthio, cyano, CO₂—H or CO—OC₁-C₄-alkyl, represents C₃-C₈-cycloalkyl or C₅-C₈-cycloalkenyl, each of which is optionally mono- or polysubstituted by fluorine, chlorine, C₁-C₄-alkyl, C₁-C₄-haloalkyl, C₁-C₄-alkoxy, CN, CO₂H or CO—OC—C₄-alkyl and each of which may optionally be interrupted by an oxygen or sulphur atom,
   represents phenyl, pyridyl, thiazolyl, pyrimidyl, benzyl, phenethyl, pyridinylmethyl or thiazolylmethyl, each of which is optionally mono- to trisubstituted by fluorine, chlorine, bromine, C₁-C₄-alkyl, C₁-C₄-haloalkyl, C₁-C₄-alkoxy, C₁-C₄-haloalkoxy, nitro or cyano;
R⁵ preferably represents hydrogen, represents C₁-C₁₀-alkyl, C₁-C₁₀-alkoxy, C₃-C₁₀-alkenyl, C₃-C₁₀-alkenyloxy or C₃-C₁₀-alkynyl, each of which is optionally mono- or polysubstituted by fluorine and/or chlorine; represents the groups CO—R⁷, S(O)₁₋₂R⁷, cyano, COOR⁷,

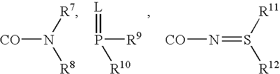

or represents pyridine, pyrimidine, triazine, thiazole or pyrazole, each of which is optionally mono- or disubstituted by fluorine, chlorine, bromine, C₁-C₄-alkyl, C₁-C₄-alkoxy, cyano or nitro; or
R⁴ and R⁵ together with the nitrogen atom to which they are attached preferably represent a saturated or unsaturated 5- or 6-membered ring which is optionally mono- or polysubstituted by C₁-C₄-alkyl and which may optionally be interrupted by oxygen or sulphur;
R⁶ preferably represents hydrogen, cations from the group of the alkali metals or alkaline earth metals or ammonium ions NH₄, mono-(C₁-C₁₀)-alkylammonium, di-(C₁-C₁₀)-alkyl-ammonium, tri-(C₁-C₁₀)-alkylammonium, tetra-(C₁-C₁₀)-alkylammonium, where the alkyl radicals of the ammonium ions may be substituted by aryl or hydroxyl, or cholinium,
   represents C₁-C₁₀-alkyl, C₃-C₁₀-alkenyl or C₃-C₁₀-alkynyl, each of which is optionally mono- or polysubstituted by fluorine, chlorine, C₁-C₄-alkoxy, C₁-C₄-alkylthio, cyano, CO₂—H or CO—OC₁-C₄-alkyl, represents C₃-C₈-cycloalkyl, C₃-C₆-cycloalkyl-C₁-C₂-alkyl or C₅-C₈-cycloalkenyl, each of which is optionally mono- or polysubstituted by fluorine, chlorine, C₁-C₄-alkyl, C₁-C₄-haloalkyl, C₁-C₄-alkoxy, CN, CO₂H or CO—OC₁-C₄-alkyl and each of which may optionally be interrupted by an oxygen or sulphur atom,
   represents phenyl, pyridyl, thiazolyl, pyrimidyl, benzyl, phenethyl, pyridinylmethyl or thiazolylmethyl, each of which is optionally mono- to trisubstituted by fluorine, chlorine, bromine, C₁-C₄-alkyl, C₁-C₄-haloalkyl, C₁-C₄-alkoxy, C₁-C₄-haloalkoxy, nitro or cyano;
R⁷ preferably represents hydrogen, represents C₁-C₁₀-alkyl, C₃-C₁₀-alkenyl or C₃-C₁₀-alkynyl, each of which is optionally mono- or polysubstituted by fluorine, chlorine, C₁-C₄-alkoxy, C₁-C₄-alkylthio, cyano, CO₂—H or CO—OC₁-C₄-alkyl, represents C₃-C₈-cycloalkyl, C₃-C₆-cycloalkyl-C₁-C₂-alkyl or C₅-C₈-cycloalkenyl, each of which is optionally mono- or polysubstituted by fluorine, chlorine, C₁-C₄-alkyl, C₁-C₄-haloalkyl, C₁-C₄-alkoxy, CN, CO₂H or CO—OC₁-C₄-alkyl and each of which may optionally be interrupted by an oxygen or sulphur atom,
   represents phenyl, pyridyl, thiazolyl, pyrimidyl, benzyl, phenethyl, pyridinylmethyl or thiazolylmethyl, each of which is optionally mono- to trisubstituted by fluorine, chlorine, bromine, C₁-C₄-alkyl, C₁-C₄-haloalkyl, C₁-C₄-alkoxy, C₁-C₄-haloalkoxy, nitro or cyano;
R⁸ preferably represents hydrogen, represents C₁-C₆-alkyl, C₃-C₆-alkenyl, C₃-C₆-alkynyl or C₁-C₆-alkoxy, each of which is optionally mono- or polysubstituted by fluorine and/or chlorine;
or
R⁴ and R⁷ together with the N—CO or N—S(O)₁₋₂ group to which they are attached preferably form a 4- to 8-membered cycle which may contain one or more heteroatoms from the group consisting of sulphur, oxygen and nitrogen, where oxygen atoms must not be adjacent to one another;
or
R⁷ and R⁸ together with the nitrogen atom to which they are attached preferably represent a saturated or unsaturated 5- or 6-membered ring which is optionally mono- or polysubstituted by C₁-C₄-alkyl and which may optionally be interrupted by oxygen or sulphur;
R⁹ and R¹⁰ preferably independently of one another represent C₁-C₈-alkyl, C₃-C₈-alkenyl, C₁-C₈-alkoxy, C₃-C₈-alkenyloxy, C₃-C₈-alkynyloxy, C₁-C₆-alkylthio, C₃-C₆-alkenylthio, each of which is optionally mono- or polysubstituted by fluorine and/or chlorine, C₃-C₈-cycloalkoxy, C₄-C₈-cycloalkenyloxy or C₃-C₈-cycloalkyl-C₁-C₂-alkoxy, each of which is optionally mono- or disubstituted by fluorine, chlorine, C₁-C₄-alkyl, C₁-C₄-haloalkyl, C₁-C₄-alkoxy, represent phenoxy, phenylthio, benzyloxy, benzylthio, pyridinyloxy, pyrazolyloxy, pyridinylthio, pyrimidylthio, thiazolylthio, pyridyl-C₁-C₂-alkoxy, pyrimidyl-C₁-C₂-alkyloxy, thiazolyl-C₁-C₂-alkyloxy, pyridyl-C₁-C₂-alkylthio, thiazolyl-C₁-C₂-alkylthio or pyrimidyl-C₁-C₂-alkylthio, each of which is optionally mono- to trisubstituted by fluorine, chlorine, bromine, C₁-C₆-alkyl-C₁-C₄-haloalkyl, C₁-C₆-alkoxy, C₁-C₄-haloalkoxy, C₁-C₆-alkylthio, cyano or nitro;
or
R⁹ and R¹⁰ together with the phosphorus atom to which they are attached preferably represent a five- to seven-membered cycle which is optionally mono- to trisubstituted by fluorine, chlorine, C₁-C₄-alkyl, C₁-C₄-alkoxy or C₁-C₄-haloalkyl and which may be interrupted by one or two oxygen and/or sulphur atoms;
R¹¹ and R¹² preferably independently of one another represent C₁-C₈-alkyl, C₃-C₈-alkenyl or C₃-C₈-alkynyl, each of which is optionally mono- to trisubstituted by fluorine or chlorine, represent phenyl or benzyl, each of which is optionally mono- or disubstituted by fluorine, chlorine, bromine, C₁-C₄-alkyl, C₁-C₄-haloalkyl, C₁-C₄-alkoxy, C₁-C₄-haloalkoxy, cyano or nitro.

The formula (I) provides a general definition of the compounds according to the invention. Hereinbelow, particularly preferred substituents, fragments or ranges of the radicals listed in the formulae mentioned above and below are illustrated for a particularly preferred embodiment of the present invention:
A particularly preferably represents a nitrogen atom, in which case the bond in the pyrimidine ring is a single bond, or represents a carbon atom, in which case the bond in the pyridine ring is a double bond,
B¹ particularly preferably represents a nitrogen atom —N═ or represents the fragment

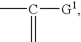

B² particularly preferably represents a nitrogen atom —N═ or represents the fragment

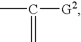

B³ particularly preferably represents a nitrogen atom —N═ or represents the fragment

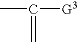

or represents a bond,
B⁴ particularly preferably represents the fragment

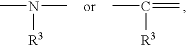

where the dashed bonds may be single bonds, double bonds or aromatic bonds;
X particularly preferably represents hydrogen, fluorine, chlorine, cyano, C₁-C₄-alkyl or C₁-C₂-haloalkyl which may be substituted by one to five fluorine and/or chlorine atoms;
Y particularly preferably represents C₁-C₈-alkyl, C₂-C₆-alkenyl, C₂-C₆-alkynyl or C₃-C₈-cycloalkyl, each of which is optionally substituted by one to five fluorine- and/or chlorine atoms;
or
Y particularly preferably represents phenyl which may optionally be mono- to trisubstituted by identical or different substituents from the group consisting of fluorine, chlorine, bromine, iodine, cyano, nitro, formyl, methyl, ethyl, n- or i-propyl, n-, s- or t-butyl, vinyl, 1-propenyl, ethynyl, 1-propynyl, allyl, propargyl, methoxy, ethoxy, n- or i-propoxy, methylthio, ethylthio, n- or i-propylthio, methylsulphinyl, ethylsulphinyl, methylsulphonyl, ethylsulphonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulphinyl, trifluoromethylsulphonyl, trichloroethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl, ethoximinoethyl, cyclopropyl, cyclobutyl, cyclopentyl and cyclohexyl, or by
   2,3- or 3,4-attached 1,3-propanediyl, 1,4-butanediyl, methylenedioxy (—O—CH₂—O—) or 1,2-ethylenedioxy (—O—CH₂—CH₂—O—), where these radicals may be mono- or polysubstituted by identical or different substituents from the group consisting of fluorine, chlorine, methyl, ethyl, n-propyl, i-propyl, trifluoromethyl, carboxyl and carboxymethyl; or
Y particularly preferably represents pyridyl which is attached in the 2- or 4-position and may be mono- to tetrasubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
   thiazolyl which is attached in the 2-, 4- or 5-position and may be mono- or disubstituted by identical or different substituents from the group consisting of fluorine, chlorine, bromine, cyano, nitro, hydroxyl, mercapto, methyl, ethyl, methoxy, methylthio, hydroximinomethyl, hydroximinoethyl, methoximinomethyl, methoximinoethyl, trifluoromethyl, carboxyl and carboxymethyl or
   pyrimidyl which is attached in the 2- or 4-position and may be mono- to trisubstituted by identical or different substituents from the group consisting of fluorine, chlorine, bromine, cyano, nitro, hydroxyl, mercapto, methyl, ethyl, methoxy, methylthio, hydroximinomethyl, hydroximinoethyl, methoximinomethyl, methoximinoethyl, trifluoromethyl, carboxyl and carboxymethyl or
   thienyl which is attached in the 2- or 4-position and may be mono- to trisubstituted by identical or different substituents from the group consisting of fluorine, chlorine, bromine, cyano, nitro, hydroxyl, mercapto, methyl, ethyl, methoxy, methylthio, hydroximinomethyl, hydroximinoethyl, methoximinomethyl, methoximinoethyl, trifluoromethyl, carboxyl and carboxymethyl;
Z particularly preferably represents chlorine, bromine, represents straight-chain or branched C₁-C₄-alkoxy, C₁-C₄-alkylthio, each of which is optionally substituted by 1 to 3 halogen atoms, or represents the group

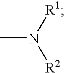

G¹, G² and G³ particularly preferably independently of one another represent hydrogen, halogen, (C₁-C₄)-alkyl which is optionally substituted by one or more halogen atoms, cyclopropyl which is optionally substituted by one of more halogen atoms; SCH₃, SC₂H₅, SOCH₃, SOC₂H₅, SO₂CH₃, SO₂C₂H₅, OCH₃ or OC₂H₅;
L particularly preferably represents oxygen or sulphur;
R¹ particularly preferably represents hydrogen, alkyl having 1 to 8 carbon atoms which is unsubstituted or mono- to trisubstituted by identical or different substituents from the group consisting of fluorine, chlorine, cyano, alkoxy having 1 to 3 carbon atoms, cycloalkyl having 3 to 6 carbon atoms, alkylthio having 1 to 3 carbon atoms; or
R¹ particularly preferably represents alkenyl having 3 to 8 carbon atoms which is unsubstituted or mono- to trisubstituted by identical or different substituents from the group consisting of fluorine, chlorine, cyano, alkoxy having 1 to 3 carbon atoms, cycloalkyl having 3 to 6 carbon atoms, alkylthio having 1 to 3 carbon atoms; or
R¹ particularly preferably represents alkynyl having 3 to 8 carbon atoms which is unsubstituted or mono- to trisubstituted by identical or different substituents from the group consisting of fluorine, chlorine, cyano, alkoxy having 1 to 3 carbon atoms, cycloalkyl having 3 to 6 carbon atoms, alkylthio having 1 to 3 carbon atoms; or
R¹ particularly preferably represents cycloalkyl having 3 to 8 carbon atoms which is unsubstituted or mono- to trisubstituted by identical or different substituents from the group consisting of fluorine, chlorine and alkyl, haloalkyl, alkoxy having in each case 1 to 2 carbon atoms; or
R¹ particularly preferably represents saturated or unsaturated heterocyclyl having 3 to 8 ring members and 1 to 2 heteroatoms, such as nitrogen, oxygen and/or sulphur, where the heterocyclyl is unsubstituted or mono- or disubstituted by hydroxyl, fluorine, chlorine, alkyl having 1 to 3 carbon atoms, alkoxy having 1 to 3 carbon atoms, cyano, nitro or cycloalkyl having 3 to 6 carbon atoms;
R² particularly preferably represents hydrogen or alkyl having 1 to 6 carbon atoms; or
R¹ and R² together with the nitrogen atom to which they are attached particularly preferably represent a saturated or unsaturated heterocyclic ring having 3 to 6 ring members, where the heterocycle optionally contains a further nitrogen, oxygen or sulphur atom as ring member and where the heterocycle may be unsubstituted or up to trisubstituted by fluorine, chlorine, bromine, alkyl having 1 to 3 carbon atoms, haloalkyl having 1 to 3 carbon atoms and 1 to 5 fluorine and/or chlorine atoms, alkoxy having 1 to 3 carbon atoms, haloalkoxy having 1 to 3 carbon atoms and 1 to 5 fluorine and/or chlorine atoms, thioalkyl having 1 to 3 carbon atoms and/or haloalkylthio having 1 to 3 carbon atoms and 1 to 5 fluorine and/or chlorine atoms;
R³ particularly preferably represents

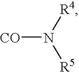

### CO—OR⁶, CO—SR⁶, CS—OR⁶ or CS—SR⁶;

R⁴ particularly preferably represents hydrogen, Na⁺, K⁺, ½Ca²⁺, ½Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺, N(CH₃)₃—H₂—C₆H₅⁺,

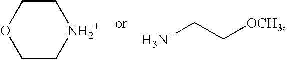

represents C₁-C₈-alkyl, C₃-C₈-alkenyl or C₃-C₈-alkynyl, each of which is optionally mono- to trisubstituted by fluorine, chlorine, C₁-C₃-alkoxy, C₁-C₃-alkylthio, cyano, CO₂H or CO—O—C₁-C₃-alkyl, represents C₃-C₆-cycloalkyl, C₃-C₆-cycloalkylmethyl or C₅-C₆-cycloalkenyl, each of which is optionally mono- to trisubstituted by fluorine, chlorine, C₁-C₃-alkyl, C₁-C₂-haloalkyl, C₁-C₃-alkoxy, CN, CO₂H or CO—O—C₁-C₃-alkyl and each of which may optionally be interrupted by an oxygen or sulphur atom,
   represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, C₁-C₃-alkyl, C₁-C₃-haloalkyl, C₁-C₃-alkoxy, C₁-C₃-haloalkoxy, nitro or cyano;
R⁵ particularly preferably represents hydrogen, represents C₁-C₈-alkyl, C₁-C₈alkoxy, C₃-C₈-alkenyl, C₃-C₈-alkenyloxy or C₃-C₈alkynyl, each of which is optionally mono- to trisubstituted by fluorine and/or chlorine; represents the groups COR⁷, S(O)₁₋₂R⁷, cyano, COOR⁷,

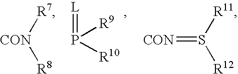

or represents pyridine, pyrimidine, triazine, thiazole, pyrazole, oxazole or triazole, each of which is optionally mono- or disubstituted by fluorine, chlorine, bromine, C₁-C₃-alkyl, C₁-C₃-alkoxy, cyano, hydroxyl or nitro; or
R⁴ and R⁵ together with the nitrogen atom to which they are attached particularly preferably represent a saturated or unsaturated 5- or 6-membered ring which is optionally mono- or disubstituted by C₁-C₂-alkyl and which may optionally be interrupted by oxygen or sulphur;
R⁶ particularly preferably represents hydrogen, Na⁺, K⁺, ½Ca²⁺, ½Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺ or N(CH₃)₃—H₂—C₆H₅⁺,
   represents C₁-C₈-alkyl, C₁-C₈-alkenyl or C₁-C₈-alkynyl, each of which is optionally mono- to trisubstituted by fluorine, chlorine, C₁-C₃-alkoxy, C₁-C₃-alkylthio, cyano, CO₂H or CO—O—C₁-C₃-alkyl, represents C₃-C₆-cycloalkyl, C₃-C₆-cycloalkylmethyl or C₅-C₆-cycloalkenyl, each of which is optionally mono- to trisubstituted by fluorine, chlorine, C₁-C₃-alkyl, C₁-C₃-haloalkyl, C₁-C₃-alkoxy, CN, CO₂H or CO—O—C₁-C₃-alkyl and each of which may optionally be interrupted by an oxygen or sulphur atom,
   represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, C₁-C₄-alkyl, C₁-C₄-haloalkyl, C₁-C₄-alkoxy, C₁-C₄-haloalkoxy, nitro or cyano;
R⁷ particularly preferably represents hydrogen, represents C₁-C₈-alkyl, C₃-C₈-alkenyl or C₃-C₈-alkynyl, each of which is optionally mono- to pentasubstituted by fluorine, chlorine, C₁-C₃-alkoxy, C₁-C₃-alkylthio, cyano, CO₂H or CO—O—C₁-C₃-alkyl, represents C₃-C₆-cycloalkyl, C₃-C₆-cycloalkylmethyl or C₅-C₆-cycloalkenyl, each of which is optionally mono- to trisubstituted by fluorine, chlorine, C₁-C₄-alkyl, C₁-C₂-haloalkyl, C₁-C₂-alkoxy, CN, CO₂H or CO—O—C₁-C₂-alkyl and each of which may optionally be interrupted by an oxygen or sulphur atom,
   represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, C₁-C₃-alkyl, C₁-C₃-haloalkyl, C₁-C₃-alkoxy, C₁-C₃-haloalkoxy, nitro or cyano;
R⁸ particularly preferably represents hydrogen, C₁-C₄-alkyl, C₃-C₄-alkenyl, C₃-C₄-alkynyl or C₁-C₄-alkoxy, each of which is optionally mono- to trisubstituted by fluorine and/or chlorine;
or
R⁴ and R⁷ together with the N—CO or N—S(O)₁₋₂ group to which they are attached particularly preferably form a 4- to 6-membered cycle which may contain one or more heteroatoms from the group consisting of sulphur, oxygen and nitrogen, where oxygen atoms must not be adjacent to one another; or
R⁷ and R⁸ together with the nitrogen atom to which they are attached particularly preferably represent a saturated or unsaturated 5- or 6-membered ring which is optionally mono- or disubstituted by C₁-C₂-alkyl and which may optionally be interrupted by oxygen or sulphur;
R⁹ and R¹⁰ independently of one another particularly preferably represent C₁-C₆-alkyl, C₃-C₆-alkenyl, C₁-C₆-alkoxy, C₃-C₆-alkenyloxy, C₃-C₆-alkynyloxy, C₁-C₄-alkylthio, C₃-C₄-alkenylthio, each of which is optionally mono- to trisubstituted by fluorine and/or chlorine, C₃-C₆-cycloalkoxy, C₄-C₆-cycloalkenyloxy or C₃-C₆-cycloalkyl-C₁-C₂-alkyloxy, each of which is optionally mono- or disubstituted by fluorine, chlorine, C₁-C₂-alkyl, C₁-C₂-haloalkyl, C₁-C₂-alkoxy, represent phenoxy, phenylthio, benzyloxy, benzylthio, pyridinyloxy, pyrazolyloxy, pyridinylthio, pyrimidylthio, thiazolylthio, pyridylmethyloxy, pyrimidylmethyloxy, thiazolylmethyloxy, pyridylmethylthio, thiazolylmethylthio or pyrimidylmethylthio, each of which is optionally mono- or disubstituted by fluorine, chlorine, bromine, C₁-C₄-alkyl-C₁-C₂-haloalkyl, C₁-C₄-alkoxy, C₁-C₂-haloalkoxy, C₁-C₄-alkylthio, cyano or nitro; or
R⁹ and R¹⁰ together with the phosphorus atom to which they are attached particularly preferably represent a five- or six-membered cycle which is optionally mono- or disubstituted by fluorine, chlorine, C₁-C₂-alkyl, C₁-C₂-alkoxy or C₁-C₂-haloalkyl and which may be interrupted by one or two oxygen and/or sulphur atoms; and
R¹¹ and R¹² independently of one another particularly preferably represent C₁-C₆-alkyl, C₃-C₆-alkenyl or C₃-C₆-alkynyl, each of which is optionally mono- to trisubstituted by fluorine or chlorine.

The formula (I) provides a general definition of the compounds according to the invention. Hereinbelow, very particularly preferred substituents, fragments or ranges of the radicals listed in the formulae mentioned above and below are illustrated for a very particularly preferred embodiment of the present invention; very particular preference is given here to the compounds of the formulae (I-1) to (I-3) which correspond to the formula (I), where the symbols A, B¹, B², B³ and B⁴ have the following very particularly preferred meanings:

For formula (I-1),

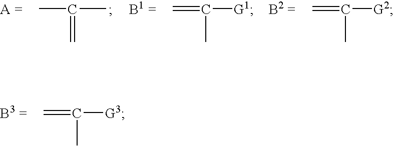

for formula (I-2)

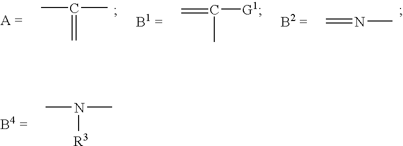

and B³ is a bond;

for formula (I-3),

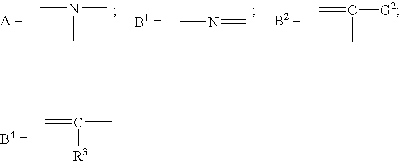

and B³ is a bond;

The other symbols in the formulae (I-1) to (I-3) have the very particularly preferred meanings below:
X very particularly preferably represents hydrogen, chlorine, methyl or trifluoromethyl;
Y very particularly preferably represents (C₁-C₆)-alkyl, (C₃-₆)-alkenyl, (C₃-C₆)-alkynyl, (C₃-C₆)-cycloalkyl, where Y is unsubstituted or substituted by one to three fluorine or chlorine atoms; or
Y very particularly preferably represents mono- to trisubstituted phenyl having substituents from the group consisting of fluorine, chlorine, bromine, cyano, nitro, methyl, ethyl, tert-butyl, methoxy, ethoxy, trifluoromethyl, difluoromethoxy and trifluoromethoxy; or
Y very particularly preferably represents pyridyl which is attached in the 2- or 4-position and which may be mono- or disubstituted by identical or different substituents from the group consisting of fluorine, chlorine, bromine, cyano, methyl, ethyl, methoxy, methylthio and trifluoromethyl; or
   pyrimidyl which is attached in the 4-position and which may be mono- to trisubstituted by identical or different substituents from the group consisting of fluorine, chlorine, bromine, cyano, methyl, ethyl, methoxy, methylthio and trifluoromethyl; or
   thienyl which is attached in the 2- or 3-position and which may be mono- or disubstituted by identical or different substituents from the group consisting of fluorine, chlorine, bromine, cyano, nitro, methyl, ethyl, methoxy and trifluoromethyl;
Z very particularly preferably represents chlorine, methoxy, ethoxy, methylthio, ethylthio or the group

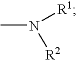

G¹, G² and G³ independently of one another very particularly preferably represent hydrogen, chlorine, bromine, methyl, ethyl, trifluoromethyl or cyclopropyl;
L very particularly preferably represents oxygen or sulphur;
R¹ very particularly preferably represents hydrogen, alkyl having 1 to 6 carbon atoms which is unsubstituted or mono- to trisubstituted by identical or different substituents from the group consisting of fluorine, chlorine, methoxy, ethoxy, cyclopropyl, cyclopentyl, cyclohexyl, methylthio and ethylthio; or
R¹ very particularly preferably represents alkenyl having 3 to 6 carbon atoms which is unsubstituted or mono- to trisubstituted by identical or different substituents from the group consisting of fluorine, chlorine, methoxy, ethoxy, methylthio and ethylthio; or
R¹ very particularly preferably represents alkynyl having 3 to 6 carbon atoms which is unsubstituted or mono- to trisubstituted by identical or different substituents from the group consisting of fluorine, chlorine, methoxy, ethoxy, methylthio and ethylthio; or
R¹ very particularly preferably represents cycloalkyl having 3 to 6 carbon atoms which is unsubstituted or mono- to trisubstituted by identical or different substituents from the group consisting of fluorine, chlorine, methyl, ethyl, methoxy and trifluoromethyl;
R² very particularly preferably represents hydrogen, methyl or ethyl; or
R¹ and R² together with the nitrogen atom to which they are attached very particularly preferably represent a saturated or unsaturated heterocyclic ring having 3 to 6 ring members, where the heterocycle optionally contains a further nitrogen, oxygen or sulphur atom as ring member and where the heterocycle may be unsubstituted or up to disubstituted by fluorine, chlorine, methyl, ethyl, trifluoromethyl, methoxy or ethoxy;
R³ very particularly preferably represents

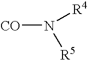

### or CO—OR⁶;

R⁴ very particularly preferably represents hydrogen, Na⁺, K⁺, ½Ca²⁺, ½Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺ or N(CH₃)₃H₂—C₆H₅⁺,
   represents C₁-C₆-alkyl, C₃-C₆-alkenyl or C₃-C₆-alkynyl, each of which is optionally mono- to trisubstituted by fluorine, chlorine, methoxy, ethoxy, methylthio, ethylthio, cyano, CO—O-methyl or CO—O-ethyl, represents C₃-C₆-cycloalkyl which is optionally mono- to trisubstituted by fluorine, chlorine, methyl, ethyl, trifluoromethyl, methoxy, ethoxy, cyano, CO—O-methyl or CO—O-ethyl;
R⁵ very particularly preferably represents hydrogen, represents C₁-C₆-alkyl, C₁-C₆-alkoxy, C₃-C₆-alkenyl, C₃-C₆-alkenyloxy or C₃-C₆-alkynyl, each of which is optionally mono- to trisubstituted by fluorine and/or chlorine, or represents the groups COR⁷, S(O)₁₋₂R⁷, cyano, COOR⁷,

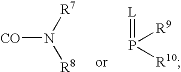

or
R⁴ and R⁵ together with the nitrogen atom to which they are attached very particularly preferably represent a saturated 5- or 6-membered ring which may optionally be interrupted by oxygen or sulphur;
R⁶ very particularly preferably represents hydrogen, Na⁺, K⁺, ½Ca²⁺, ½Mg²⁺, NH₄⁺, NH₃CH₃⁺, NH₂(CH₃)₂⁺, NH(CH₃)₃⁺, NH(C₂H₅₎₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺ or N(CH₃)₃—H₂—C₆H₅⁺,
   represents C₁-C₆-alkyl, C₃-C₆-alkenyl or C₃-C₆-alkynyl, each of which is optionally mono- to trisubstituted by fluorine, chlorine, .methoxy, ethoxy, methylthio, ethylthio, cyano, CO₂H, CO—O-methyl or CO—O-ethyl;
R⁷ very particularly preferably represents hydrogen, represents C₁-C₆-alkyl, C₃-C₆-alkenyl or C₃-C₆-alkynyl, each of which is optionally mono- to trisubstituted by fluorine, chlorine, methoxy, ethoxy, methylthio, ethylthio, cyano, CO—O-methyl or CO—O-ethyl, represents C₃-C₆-cycloalkyl, C₃-C₆-cycloalkylmethyl or C₅-C₆-cycloalkenyl, each of which is optionally monosubstituted by fluorine, chlorine, methyl, ethyl, methoxy, trifluoromethyl, cyano, CO₂H, CO—O-methyl or CO—O-ethyl and each of which may optionally be interrupted by an oxygen or sulphur atom, represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, methyl, trifluoromethyl, methoxy;
R⁸ very particularly preferably represents hydrogen, methyl, ethyl, propyl, isopropyl, allyl, propargyl, C₃-C₄-alkenyl, methoxy or ethoxy; or
R⁷ and R⁸ together with the nitrogen atom to which they are attached very particularly preferably represent a saturated 5- or 6-membered ring which may optionally be interrupted by oxygen or sulphur; and
R⁹ and R¹⁰ independently of one another very particularly preferably represent methyl, ethyl, methoxy, ethoxy, propoxy, i-propoxy, butoxy, alkyloxy, methylthio, ethylthio, propylthio, butylthio, i-propylthio, sec-butylthio, allylthio, each of which is optionally mono- to trisubstituted by fluorine and/or chlorine.

The formula (I) provides a general definition of the compounds according to the invention. Hereinbelow, especially very particularly preferred substituents, fragments or ranges of the radicals listed in the formulae mentioned above and below are illustrated for an especially very particularly preferred embodiment of the present invention; special very particular preference is given here to the compounds of the formula (I-3) which correspond to the formula (I), where the symbols A, B¹, B², B³ and B⁴ have the following especially very particularly preferred meanings:

For formula (I-3),

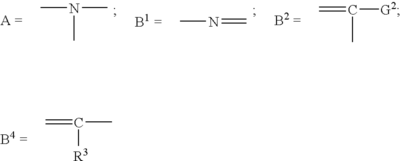

and B³ is a bond.

The other symbols in the formula (I-3) have the especially very particularly preferred meanings below:
X especially very particularly preferably represents hydrogen, chlorine, methyl or trifluoromethyl;
Y especially very particularly preferably represents (C₁-C₆)-alkyl, (C₃-₆)-alkenyl, (C₃-C₆)-alkynyl, (C₃-C₆)-cycloalkyl, where Y is unsubstituted or substituted by one to three fluorine or chlorine atoms; or
Y especially very particularly preferably represents mono- to trisubstituted phenyl having substituents from the group consisting of fluorine, chlorine, bromine, cyano, nitro, methyl, ethyl, tert-butyl, methoxy, ethoxy, trifluoromethyl, difluoromethoxy and trifluoromethoxy;
or
Y especially very particularly preferably represents pyridyl which is attached in the 2- or 4-position and may be mono- or disubstituted by identical or different substituents from the group consisting of fluorine, chlorine, bromine, cyano, methyl, ethyl, methoxy, methylthio and trifluoromethyl; or
   pyrimidyl which is attached in the 4-position and may be mono- to trisubstituted by identical or different substituents from the group consisting of fluorine, chlorine, bromine, cyano, methyl, ethyl, methoxy, methylthio and trifluoromethyl; or
   thienyl which is attached in the 2- or 3-position and may be mono- or disubstituted by identical or different substituents from the group consisting of fluorine, chlorine, bromine, cyano, nitro, methyl, ethyl, methoxy and trifluoromethyl;
Z especially very particularly preferably represents chlorine, methoxy, ethoxy, methylthio, ethylthio or the group

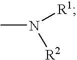

G¹, G² and G³ especially very particularly preferably independently of one another represent hydrogen, chlorine, bromine, methyl, ethyl, trifluoromethyl or cyclopropyl;
L especially very particularly preferably represents oxygen or sulphur;
R¹ especially very particularly preferably represents hydrogen, alkyl having 1 to 6 carbon atoms which is unsubstituted or mono- to trisubstituted by identical or different substituents from the group consisting of fluorine, chlorine, methoxy, ethoxy, cyclopropyl, cyclopentyl, cyclohexyl, methylthio and ethylthio; or
R¹ especially very particularly preferably represents alkenyl having 3 to 6 carbon atoms which is unsubstituted or mono- to trisubstituted by identical or different substituents from the group consisting of fluorine, chlorine, methoxy, ethoxy, methylthio and ethylthio; or
R¹ especially very particularly preferably represents alkynyl having 3 to 6 carbon atoms which is unsubstituted or mono- to trisubstituted by identical or different substituents from the group consisting of fluorine, chlorine, methoxy, ethoxy, methylthio and ethylthio; or
R¹ especially very particularly preferably represents cycloalkyl having 3 to 6 carbon atoms which is unsubstituted or mono- to trisubstituted by identical or different substituents from the group consisting of fluorine, chlorine, methyl, ethyl, methoxy and trifluoromethyl;
R² especially very particularly preferably represents hydrogen, methyl or ethyl; or
R¹ and R² especially very particularly preferably together with the nitrogen atom to which they are attached represent a saturated or unsaturated heterocyclic ring having 3 to 6 ring members, where the heterocycle optionally contains a further nitrogen, oxygen or sulphur atom as ring member and where the heterocycle may be unsubstituted or up to disubstituted by fluorine, chlorine, methyl, ethyl, trifluoromethyl, methoxy or ethoxy;
R³ especially very particularly preferably represents

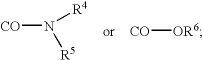

R⁴ especially very particularly preferably represents hydrogen,
   represents C₁-C₆-alkyl, C₃-C₆-alkenyl or C₃-C₆-alkynyl, each of which is optionally mono- to trisubstituted by fluorine, chlorine, methoxy, ethoxy, methylthio, ethylthio, cyano, CO—O-methyl or CO—O-ethyl, represents C₃-C₆-cycloalkyl which is optionally mono- to trisubstituted by fluorine, chlorine, methyl, ethyl, trifluoromethyl, methoxy, ethoxy, cyano, CO—O-methyl or CO—O-ethyl;
R⁵ especially very particularly preferably represents hydrogen, represents C₁-C₆-alkyl, C₁-C₆-alkoxy, C₃-C₆-alkenyl, C₃-C₆-alkenyloxy or C₃-C₆-alkynyl, each of which is optionally mono- to trisubstituted by fluorine and/or chlorine, or represents the groups COR⁷, S(O)₁₋₂R⁷, cyano, COOR⁷,

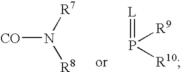

or
R⁴ and R⁵ together with the nitrogen atom to which they are attached especially very particularly preferably represent a saturated 5- or 6-membered ring which may optionally be interrupted by oxygen or sulphur;
R⁶ especially very particularly preferably represents hydrogen,
   represents C₁-C₆-alkyl, C₃-C₆-alkenyl or C₃-C₆-alkynyl, each of which is optionally mono- to trisubstituted by fluorine, chlorine, methoxy, ethoxy, methylthio, ethylthio, cyano, CO₂H, CO—O-methyl or CO—O-ethyl;
R⁷ especially very particularly preferably represents hydrogen, represents C₁-C₆-alkyl, C₃-C₆-alkenyl or C₃-C₆-alkynyl, each of which is optionally mono- to trisubstituted by fluorine, chlorine, methoxy, ethoxy, methylthio, ethylthio, cyano, CO—O-methyl or CO—O-ethyl, represents C₃-C₆-cycloalkyl, C₃-C₆-cycloalkylmethyl or C₅-C₆-cycloalkenyl, each of which is optionally monosubstituted by fluorine, chlorine, methyl, ethyl, methoxy, trifluoromethyl, cyano, CO₂H, CO—O-methyl or CO—O-ethyl and each of which may optionally be interrupted by an oxygen or sulphur atom, represents phenyl, pyridyl, thiazolyl, pyrimidyl, benzyl, phenethyl, pyridinylmethyl or thiazolylmethyl, each of which is optionally mono- to disubstituted by fluorine, chlorine, bromine, methyl, trifluoromethyl, methoxy;
R⁸ especially very particularly preferably represents hydrogen, methyl, ethyl, propyl, isopropyl, allyl, propargyl, C₃-C₄-alkenyl, methoxy or ethoxy; or
R⁷ and R⁸ together with the nitrogen atom to which they are attached especially very particularly preferably represent a saturated 5- or 6-membered ring which may optionally be interrupted by oxygen or sulphur; and
R⁹ and R¹⁰ independently of one another especially very particularly preferably represent methyl, ethyl, methoxy, ethoxy, propoxy, i-propoxy, butoxy, alkyloxy, methylthio, ethylthio, propylthio, butylthio, i-propylthio, sec-butylthio, allylthio, each of which is optionally mono- to trisubstituted by fluorine and/or chlorine.

The general or preferred radical definitions or illustrations listed above can be combined with one another as desired, i.e. including combinations between the respective ranges and preferred ranges. They apply to the end products and, correspondingly, to precursors and intermediates.

Preference according to the invention is given to the compounds of the formula (I) which contain a combination of the meanings listed above as being preferred (preferable).

Particular preference according to the invention is given to the compounds of the formula (I) which contain a combination of the meanings listed above as being particularly preferred.

Very particular preference according to the invention is given to the compounds of the formula (I) which contain a combination of the meanings listed above as being very particularly preferred.

Special particular preference according to the invention is given to the compounds of the formula (I) which contain a combination of the meanings listed above as being especially particularly preferred.

Special very particular preference according to the invention is given to the compounds of the formula (I) which contain a combination of the meanings listed above as being especially very particularly preferred.

Saturated or unsaturated hydrocarbon radicals, such as alkyl or alkenyl, can in each case be straight-chain or branched as far as this is possible, including in combination with heteroatoms, such as, for example, in alkoxy.

Unless indicated otherwise, optionally substituted radicals may be mono- or polysubstituted, where in the case of polysubstitution the substituents can be identical or different.

In the literature it has already been described how the action of various active compounds can be boosted by addition of ammonium salts. The salts in question, however, are detersive salts (for example WO 95/017817) or salts which have relatively long alkyl substituents and/or aryl substituents and which have a permeabilizing action or which increase the active compound's solubility (for example EP-A 0 453 086, EP-A 0 664 081, FR-A 2 600 494, U.S. Pat. No. 4,844,734, U.S. Pat. No. 5,462,912, U.S. Pat. No. 5,538,937, US-A 03/0224939, US-A 05/0009880, US-A 05/0096386). Moreover, the prior art describes the action only for particular active compounds and/or particular applications of the corresponding compositions. In other cases, they are salts of sulphonic acids where the acids for their part have paralysing action on the insects (U.S. Pat. No. 2,842,476). A boost of action by ammonium sulphate, for example, is described by way of example for the herbicides glyphosate and phosphinothricin (U.S. Pat. No. 6,645,914, EP-A2 0 036 106). However, this prior art neither discloses nor suggests a corresponding action for insecticides.

The use of ammonium sulphate as formulating assistant has also been described for certain active compounds and applications (WO 92/16108), but its purpose therein is to stabilize the formulation, not to boost the action.

It has now been found, entirely surprisingly, that the action of insecticides and/or acaricides from the class of the heterocyclic carboxylic acid derivatives (I) can be boosted significantly through the addition of ammonium salts or phosphonium salts to the application solution or through the incorporation of these salts into a formulation comprising heterocyclic carboxylic acid derivatives (I). The present invention therefore provides for the use of ammonium salts or phosphonium salts for boosting the action of crop protection compositions which comprise as their active compound insecticidal and/or acaricidal heterocyclic carboxylic acid derivatives (I). The invention likewise provides compositions which comprise insecticidal and/or acaricidal heterocyclic carboxylic acid derivatives (I) and action-boosting ammonium salts or phosphonium salts, including not only formulated active compounds but also ready-to-use compositions (spray liquors). The invention further provides, finally, for the use of these compositions for controlling harmful insects and/or spider mites.

Ammonium salts and phosphonium salts which inventively boost the activity of crop protection compositions comprising heterocyclic carboxylic acid derivatives (I) are defined by formula (II)

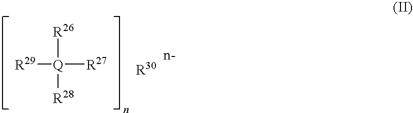

in which
Q represents nitrogen or phosphorus,
Q preferably represents nitrogen,
R²⁶, R²⁷, R²⁸ and R²⁹ independently of one another represent hydrogen or in each case optionally substituted C₁-C₈-alkyl or mono- or polyunsaturated, optionally substituted C₁-C₈-alkylene, the substituents being selectable from halogen, nitro and cyano,
R²⁶, R²⁷, R²⁸ and R²⁹ independently of one another preferably represent hydrogen or in each case optionally substituted C₁-C₄-alkyl, the substituents being selectable from halogen, nitro and cyano,
R²⁶, R²⁷, R²⁸ and R²⁹ independently of one another particularly preferably represent hydrogen, methyl, ethyl, n-propyl, isopropyl, n-butyl, isobutyl, sec-butyl or tert-butyl,
R²⁶, R²⁷, R²⁸ and R²⁹ very particularly preferably represent hydrogen,
R²⁶, R²⁷, R²⁸ and R²⁹ furthermore very particularly preferably all represent methyl or all represent ethyl,
n represents 1, 2, 3 or 4,
n preferably represents 1 or 2,
R³⁰ represents an organic or inorganic anion,
R³⁰ preferably represents bicarbonate, tetraborate, fluoride, bromide, iodide, chloride, monohydrogenphosphate, dihydrogenphosphate, hydrogensulphate, tartrate, sulphate, nitrate, thiosulphate, thiocyanate, formate, lactate, acetate, propionate, butyrate, pentanoate, citrate or oxalate,
R³⁰ furthermore preferably represents carbonate, pentaborate, sulphite, benzoate, hydrogenoxalate, hydrogencitrate, methylsulphate or tetrafluoroborate,
R³⁰ particularly preferably represents lactate, sulphate, nitrate, thiosulphate, thiocyanate, citrate, oxalate, acetate or formate,
R³⁰ moreover particularly preferably represents monohydrogenphosphate or dihydrogenphosphate and
R³⁰ very particularly preferably represents thiocyanate, dihydrogenphosphate, monohydrogenphosphate or sulphate.

The ammonium salts and phosphonium salts of the formula (II) can be used in a broad concentration range to boost the activity of crop protection compositions comprising heterocyclic carboxylic acid derivatives (I). In general the ammonium salts or phosphonium salts are used in the ready-to-use crop protection composition in a concentration of 0.5 to 80 mmol/l, preferably 0.75 to 37.5 mmol/1, more preferably 1.5 to 25 mmol/l. In the case of a formulated product the ammonium salt and/or phosphonium salt concentration in the formulation is chosen such that it is within these stated general, preferred or particularly preferred ranges after the formulation has been diluted to the desired active-ingredient concentration. The concentration of the salt in the formulation is typically 1%-50% by weight.

In one preferred embodiment of the invention the activity is boosted by adding to the crop protection compositions not only an ammonium salt and/or phosphonium salt but also, additionally, a penetrant. It is considered entirely surprising that even in these cases an even greater boost to activity is observed. The present invention therefore likewise provides for the use of a combination of penetrant and ammonium salts and/or phosphonium salts to boost the activity of crop protection compositions which comprise insecticidal and/or acaricidal heterocyclic carboxylic acid derivatives (I) as active compound. The invention likewise provides compositions which comprise insecticidal and/or acaricidal heterocyclic carboxylic acid derivatives (I), penetrants and ammonium salts and/or phosphonium salts, including specifically not only formulated active compounds but also ready-to-use compositions (spray liquors). The invention additionally provides, finally, for the use of these compositions for controlling harmful insects.

Suitable penetrants in the present context include all those substances which are typically used to enhance the penetration of active agrochemical compounds into plants. Penetrants are defined in this context by their ability to penetrate from the aqueous spray liquor and/or from the spray coating into the cuticle of the plant and thereby to increase the mobility of active compounds in the cuticle. The method described in the literature (Baur et al., 1997, Pesticide Science 51, 131-152) can be used in order to determine this property.

Examples of suitable penetrants include alkanol alkoxylates. Penetrants of the invention are alkanol alkoxylates of the formula

R—O—(-AO)ᵥ—R′ (III)

in which
R represents straight-chain or branched alkyl having 4 to 20 carbon atoms,
R′ represents hydrogen, methyl, ethyl, n-propyl, isopropyl, n-butyl, isobutyl, tert-butyl, n-pentyl or n-hexyl,
AO represents an ethylene oxide radical, a propylene oxide radical, a butylene oxide radical or represents mixtures of ethylene oxide and propylene oxide radicals or butylene oxide radicals, and
v represents numbers from 2 to 30.

One preferred group of penetrants are alkanol alkoxylates of the formula

R—O—(-EO—)ₙ—R′ (III-a)

in which
R has the meaning given above,
R′ has the meaning given above,
EO represents —CH₂—CH₂—O— and
n represents a number from 2 to 20.

A further preferred group of penetrants are alkanol alkoxylates of the formula

R—O—(-EO—)ₚ—(—PO—)_{q}—R′ (III-b)

in which
R has the meaning given above,
R′ has the meaning given above,
EO represents —CH₂—CH₂—O—,
PO represents

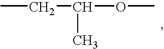

p represents a number from 1 to 10 and
q represents a number from 1 to 10.

A further preferred group of penetrants are alkanol alkoxylates of the formula

R—O—(—PO—)ᵣ-(EO—)ₛ—R′ (III-c)

in which
R has the meaning given above,
R′ has the meaning given above,
EO represents —CH₂—CH₂—O—,
PO represents

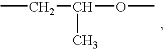

r represents a number from 1 to 10 and
s represents a number from 1 to 10.

A further preferred group of penetrants are alkanol alkoxylates of the formula

R—O—(-EO—)ₚ—(—BO—)_{q}—R′ (III-d)

in which
R and R′ have the meanings given above,
EO represents CH₂—CH₂—O—,
BO represents

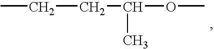

p represents a number from 1 to 10 and
q represents a number from 1 to 10.

A further preferred group of penetrants are alkanol alkoxylates of the formula

R—O—(—BO—)₄—(-EO—)ₛ—R′ (III-e)

in which
R and R′ have the meanings given above,
BO represents

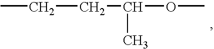

EO represents CH₂—CH₂—O—,
r represents a number from 1 to 10 and
s represents a number from 1 to 10.

A further preferred group of penetrants are alkanol alkoxylates of the formula

CH₃—(CH₂)ₜ—CH₂—O—(—CH₂—CH₂—O—)ᵤ—R′ (III-f)

in which
R′ has the meaning given above,
t represents a number from 8 to 13
u represents a number from 6 to 17.

In the formulae indicated above,

R preferably represents butyl, isobutyl, n-pentyl, isopentyl, neopentyl, n-hexyl, isohexyl, n-octyl, isooctyl, 2-ethylhexyl, nonyl, isononyl, decyl, n-dodecyl, isododecyl, lauryl, myristyl, isotridecyl, trimethylnonyl, palmityl, stearyl or eicosyl.

As an example of an alkanol alkoxylate of the formula (III-c) mention may be made of 2-ethylhexyl alkoxylate of the formula

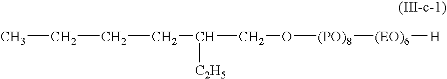

in which
EO represents —CH₂—CH₂—O—,
PO represents

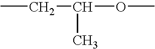

and the numbers 8 and 6 represent average values.

As an example of an alkanol alkoxylate of the formula (III-d) mention may be made of the formula

CH₃—(CH₂)₁₀—O—(-EO—)₆—(—BO—)₂—CH₃ (III-d-1)

in welcher
EO represents CH₂—CH₂—O—,
BO represents

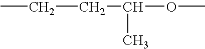

and the numbers 10, 6 and 2 represent average values.

Particularly preferred alkanol alkoxylates of the formula (III-f) are compounds of this formula in which
t represents a number from 9 to 12 and
u represents a number from 7 to 9.

Mention may be made with very particular preference of alkanol alkoxylate of the formula (III-f-1)

CH₃—(CH₂)ₜ—CH₂—O—(—CH₂—CH₂—O—)ᵤ—H (III-f-1)

in which
t stands for the average value 10.5 and
u stands for the average value 8.4.

A general definition of the alkanol alkoxylates is given by the formulae above. These substances are mixtures of compounds of the stated type with different chain lengths. The indices therefore have average values which may also deviate from whole numbers.

The alkanol alkoxylates of the formulae stated are known and in some cases are available commercially or can be prepared by known methods (cf. WO 98/35 553, WO 00/35 278 and EP-A 0 681 865).

Suitable penetrants also include, for example, substances which promote the availability of the compounds of the formula (I) in the spray coating. These include, for example, mineral or vegetable oils. Suitable oils are all mineral or vegetable oils—modified or otherwise—which can typically be used in agrochemical compositions. Mention may be made by way of example of sunflower oil, rapeseed oil, olive oil, castor oil, colza oil, maize seed oil, cotton seed oil and soybean oil, or the esters of said oils. Preference is given to rapeseed oil, sunflower oil and their methyl or ethyl esters.

The concentration of penetrant in the compositions according to invention can be varied within a wide range. In the case of a formulated crop protection composition it is in general 1% to 95%, preferably 1% to 55%, more preferably 15%-40% by weight. In the ready-to-use compositions (spray liquors) the concentrations are generally between 0.1 and 10 g/l, preferably between 0.5 and 5 g/l.

Crop protection compositions according to invention may also comprise further components, examples being surfactants and/or dispersing assistants or emulsifiers.

Suitable nonionic surfactants and/or dispersing assistants include all substances of this type that can typically be used in agrochemical compositions. Preferably mention may be made of polyethylene oxide-polypropylene oxide block copolymers, polyethylene glycol ethers of linear alcohols, reaction products of fatty acids with ethylene oxide and/or propylene oxide, and also polyvinyl alcohol, polyvinylpyrrolidone, copolymers of polyvinyl alcohol and polyvinylpyrrolidone, and copolymers of (meth)acrylic acid and (meth)acrylic esters, and additionally alkyl ethoxylates and alkylaryl ethoxylates, which optionally may be phosphated and optionally may be neutralized with bases, mention being made, by way of example, of sorbitol ethoxylates, and, as well, polyoxyalkylenamine derivatives.

Suitable anionic surfactants include all substances of this type that can typically be used in agrochemical compositions. Preference is given to alkali metal salts and alkaline earth metal salts of alkylsulphonic acids or alkylarylsulphonic acids.

A further preferred group of anionic surfactants and/or dispersing assistants are the following salts that are of low solubility in plant oil: salts of polystyrenesulphonic acids, salts of polyvinylsulphonic acids, salts of naphthalenesulphonic acid-formaldehyde condensation products, salts of condensation products of naphthalenesulphonic acid, phenolsulphonic acid and formaldehyde, and salts of lignosulphonic acid.

Suitable additives which may be included in the formulations according to invention are emulsifiers, foam inhibitors, preservatives, antioxidants, colorants and inert filling materials.

Preferred emulsifiers are ethoxylated nonylphenols, reaction products of alkylphenols with ethylene oxide and/or propylene oxide, ethoxylated arylalkylphenols, and also ethoxylated and propoxylated arylalkylphenols, and also sulphated or phosphated arylalkyl ethoxylates and/or arylalkyl ethoxypropoxylates, mention being made by way of example of sorbitan derivatives, such as polyethylene oxide-sorbitan fatty acid esters, and sorbitan fatty acid esters.

The active compounds according to the invention, in combination with good plant tolerance and favourable toxicity to warm-blooded animals and being tolerated well by the environment, are suitable for protecting plants and plant organs, for increasing the harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They may be preferably employed as plant protection agents. They are active against normally sensitive and resistant species and against all or some stages of development. The abovementioned pests include:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example, Arion spp., Biomphalaria spp., Bulinus spp., Deroceras spp., Galba spp., Lymnaea spp., Oncomelania spp., Succinea spp.

From the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control protozoa, such as Eimeria.

From the order of the heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis, Vespa spp.

From the order of the Isopoda, for example, Armadillidium vulgare, Oniscus asellus, Porcellio scaber.

From the order of the Isoptera, for example, Reticulitermes spp., Odontotermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Cheimatobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example, Scutigerella immaculata.

From the order of the Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example, Lepisma saccharina.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

The compounds of the formula (I) according to the invention are distinguished in particular by strong activity against insects, parasites from the subclass of the Acari (Acarina) (such as mites, spider mites and/or ticks) and/or nematodes.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as intermediates or precursors for the synthesis of other active compounds.

The active compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and microencapsulations in polymeric substances.

These formulations are produced in a known manner, for example by mixing the active compounds with extenders, that is liquid solvents and/or solid carriers, optionally with the use of surfactants, that is emulsifiers and/or dispersants and/or foam-formers. The formulations are prepared either in suitable plants or else before or during the application.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethyl sulphoxide, and also water.

Suitable solid carriers are:

for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE and/or -POP ethers, acid and/or POP-POE esters, alkylaryl and/or POP-POE ethers, fat- and/or POP-POE adducts, POE- and/or POP-polyol derivatives, POE- and/or POP-sorbitan- or -sugar adducts, alkyl or aryl sulphates, alkyl- or arylsulphonates and alkyl or aryl phosphates or the corresponding PO-ether adducts. Furthermore, suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

Other possible additives are perfumes, mineral or vegetable, optionally modified oils, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

The formulations generally comprise between 0.01 and 98% by weight of active compound, preferably between 0.5 and 90%.

The active compound according to the invention can be used in its commercially available formulations and in the use forms, prepared from these formulations, as a mixture with other active compounds, such as insecticides, attractants, sterilizing agents, bactericides, acaricides, nematicides, fungicides, growth-regulating substances, herbicides, safeners, fertilizers or semiochemicals.

Particularly. favourable mixing partners are, for example, the following components:

Fungicides:

Inhibitors of nucleic acid synthesis

benalaxyl, benalaxyl-M, bupirimate, chiralaxyl, clozylacon, dimethirimol, ethirimol, furalaxyl, hymexazol, mefenoxam, metalaxyl, metalaxyl-M, ofurace, oxadixyl, oxolinic acid

Inhibitors of mitosis and cell division

benomyl, carbendazim, diethofencarb, ethaboxam, fuberidazole, pencycuron, thiabendazole, thiophanate-methyl, zoxamide

Inhibitors of respiratory chain complex I

diflumetorim

Inhibitors of respiratory chain complex II

boscalid, carboxin, fenfuram, flutolanil, furametpyr, furmecyclox, mepronil, oxycarboxin, penthiopyrad, thifluzamide

Inhibitors of respiratory chain complex III

azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin, trifloxystrobin

Decouplers

dinocap, fluazinam

Inhibitors of ATP production

fentin acetate, fentin chloride, fentin hydroxide, silthiofam

Inhibitors of amino acid biosynthesis and protein biosynthesis

andoprim, blasticidiN—S, cyprodinil, kasugamycin, kasugamycin hydrochloride hydrate, mepanipyrim, pyrimethanil

Inhibitors of signal transduction

fenpiclonil, fludioxonil, quinoxyfen

Inhibitors of lipid and membrane synthesis

chlozolinate, iprodione, procymidone, vinclozolin

ampropylfos, potassium-ampropylfos, edifenphos, etridiazole, iprobenfos (IBP), isoprothiolane, pyrazophos

tolclofos-methyl, biphenyl

iodocarb, propamocarb, propamocarb hydrochloride, propamocarb-fosetylate

Inhibitors of ergosterol biosynthesis

fenhexamid,

azaconazole, bitertanol, bromuconazole, cyproconazole, diclobutrazole, difenoconazole, diniconazole, diniconazole-M, epoxiconazole, etaconazole, fenarimol, fenbuconazole, fluquinconazole, flurprimidole, flusilazole, flutriafol, furconazole, furconazole-cis, hexaconazole, imazalil, imazalil sulphate, imibenconazole, ipconazole, metconazole, myclobutanil, nuarimol, oxpoconazole, paclobutrazole, penconazole, pefurazoate, prochloraz, propiconazole, prothioconazole, pyrifenox, simeconazole, tebuconazole, tetraconazole, triadimefon, triadimenol, triflumizole, triforine, triticonazole, uniconazole, voriconazole, viniconazole,

aldimorph, dodemorph, dodemorph acetate, fenpropidin, fenpropimorph, spiroxamine, tridemorph,

naftifine, pyributicarb, terbinafine

Inhibitors of cell wall synthesis

benthiavalicarb, bialaphos, dimethomorph, flumorph, iprovalicarb, mandipropamid, polyoxins, polyoxorim, validamycin A

Inhibitors of melanin biosynthesis

capropamid, diclocymet, fenoxanil, phthalide, pyroquilon, tricyclazole

Resistance Inductors

acibenzolar-S-methyl, probenazole, tiadinil

Multisite

captafol, captan, chlorothalonil, copper salts such as: copper hydroxide, copper naphthenate, copper oxychloride, copper sulphate, copper oxide, oxine-copper and Bordeaux mixture, dichlofluanid, dithianon, dodine, dodine free base, ferbam, folpet, fluorofolpet, guazatine, guazatine acetate, iminoctadine, iminoctadine albesilate, iminoctadine triacetate, mancopper, mancozeb, maneb, metiram, metiram zinc, propineb, sulphur and sulphur preparations containing calcium polysulphide, thiram, tolylfluanid, zineb, ziram

Further Fungicides

amibromdol, benthiazole, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat metilsulphate, diphenylamine, ferimzone, flumetover, flusulfamide, fluopicolide, fluoroimide, fosetyl-aluminium, fosetyl-calcium, fosetyl-sodium, hexachlorobenzene, 8-hydroxy-quinoline sulphate, irumamycin, methasulfocarb, metrafenone, methyl isothiocyanate, mildiomycin, natamycin, nickel dimethyl dithiocarbamate, nitrothal-isopropyl, octhilinone, oxamocarb, oxyfenthiin, pentachlorophenol and salts, 2-phenylphenol and salts, piperalin, propanosine-sodium, proquinazid, pyribencarb, pyrrolnitrin, quintozene, tecloftalam, tecnazene, triazoxide, trichlamide, valiphenal, zarilamid,
2-(2-{[6-(3-chloro-2-methylphenoxy)-5-fluoropyrimidin-4-yl]oxy}phenyl)-2-(methoxyimino)-N-methylacetamide,
2-[[[[1-[3-(1-fluoro-2-phenylethyl)oxy]phenyl]ethylidene]amino]oxy]methyl]-alpha-(methoxyimino)-N-methyl-alpha-benzacetamide,
cis-1-(4-chlorophenyl)-2-(1H-1,2,4-triazol-1-yl)cycloheptanol,
1-[(4-methoxyphenoxy)methyl]-2,2-dimethylpropyl-1H-imidazole-1-carboxylic acid,
2,3,5,6-tetrachloro-4-(methylsulphonyl)pyridine,
2-butoxy-6-iodo-3-propylbenzopyranon-4-one,
2-chloro-N-(2,3-dihydro-1,1,3-dimethyl-1H-inden-4-yl)-3-pyridinecarboxamide,
3,4,5-trichloro-2,6-pyridinedicarbonitrile,
3,4-dichloro-N-(2-cyanophenyl)isothiazole-5-carboxamide (isotianil)
3-[5-(4-chlorophenyl)-2,3-dimethylisoxazolidin-3-yl]pyridine,
5-chloro-6-(2,4,6-trifluorophenyl)-N-[(1R)-1,2,2-trimethylpropyl][1,2,4]triazolo[1,5-a[pyrimidine-7-amine,
5-chloro-7-(4-methylpiperidin-1-yl)-6-(2,4,6-trifluorophenyl)[1,2,4]triazolo[1,5-a]pyrimidine,
5-chloro-N-[(1R)-1,2-dimethylpropyl]-6-(2,4,6-trifluorophenyl)[1,2,4]triazolo[1,5-a]pyrimidine-7-amine,
methyl 2-[[[cyclopropyl[(4-methoxyphenyl)imino]methyl]thio]methyl]-alpha-(methoxymethylene)benzacetate,
methyl 1-(2,3-dihydro-2,2-dimethyl-1H-inden-1-yl)-1H-imidazole-5-carboxylate,
N-(3′,4′-dichloro-5-fluorobiphenyl-2-yl)-3-(difluoromethyl)-1-methyl-1H-pyrazole-4-carboxamide,
N-(3-ethyl-3,5,5-trimethylcyclohexyl)-3-formylamino-2-hydroxybenzamide,
N-(4-chloro-2-nitrophenyl)-N-ethyl-4-methylbenzenesulphonamide,
N-(4-chlorobenzyl)-3-[3-methoxy-4-(prop-2-yn-1-yloxy)phenyl]propanamide,
N-[(4-chlorophenyl)(cyano)methyl]-3-[3-methoxy-4-(prop-2-yn-1-yloxy)phenyl]propanamide,
N-(5-bromo-3-chloropyridin-2-yl)methyl-2,4-dichloronicotinamide,
N-[1-(5-bromo-3-chloropyridin-2-yl)ethyl]-2,4-dichloronicotinamide,
(2S)-N-[2-[4-[[3-(4-chlorophenyl)-2-propynyl]oxy]-3-methoxyphenyl]ethyl]-3-methyl-2-[(methylsulphonyl)amino]butanamide,
N-{(Z)-[(cyclopropylmethoxy)imino][6-(difluoromethoxy)-2,3-difluorophenyl]methyl}-2-benzacetamide,
N-{2-[1,1′-bi(cyclopropyl)-2-yl]phenyl}-3-(difluoromethyl)-1-methyl-1H-pyrazole-4-carboxamide,
N-{2-[3-chloro-5-(trifluoromethyl)pyridin-2-yl]ethyl}-2-(trifluoromethyl)benzamide,
N-ethyl-N-methyl-N′-{2-methyl-5-(trifluoromethyl)-4-[3-(trimethylsilyl)propoxy]phenyl}-imidoformamide,
O-[1-[(4-methoxyphenoxy)methyl]-2,2-dimethylpropyl]-1H-imidazole-1-carbothioic acid,
2-amino-4-methyl-N-phenyl-5-thiazolecarboxamide,
2,4-dihydro-5-methoxy-2-methyl-4-[[[[1-[3-(trifluoromethyl)phenyl]ethylidene]amino]oxy]methyl]phenyl]-3H-1,2,4-triazol-3-one (CAS No. 185336-79-2),
N-(6-methoxy-3-pyridinyl)cyclopropanecarboxamide,

Bactericides:

bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

Insecticides/Acaricides/Nematicides:

Acetylcholine esterase (AChE) inhibitors

Carbamates,

for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb, triazamate

Organophosphates,

for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion

Sodium channel modulators/voltage-dependent sodium channel blockers

Pyrethroids,

for example acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans-isomer), prallethrin, profluthrin, protrifenbute, pyresmethrin, resmethrin, RU 15525, silafluofen, tau-fluvalinate, tefluthrin, terallethrin, tetramethrin (IR isomer), tralomethrin, transfluthrin, ZXI 8901, pyrethrins (pyrethrum)

DDT

oxadiazines,

for example indoxacarb

semicarbazones,

for example metaflumizone (BAS3201)

Acetylcholine receptor agonists/antagonists

chloronicotinyls,

for example acetamiprid, clothianidin, dinotefuran, imidacloprid, nitenpyram, nithiazine, thiacloprid, thiamethoxam

nicotine, bensultap, cartap

Acetylcholine receptor modulators

spinosyns,

for example spinosad

GABA-controlled chloride channel antagonists

organochlorines,

for example camphechlor, chlordane, endosulphan, gamma-HCH, HCH, heptachlor, lindane, methoxychlor

fiproles,

for example acetoprole, ethiprole, fipronil, pyrafluprole, pyriprole, vaniliprole

Chloride channel activators

mectins,

for example abamectin, emamectin, emamectin-benzoate, ivermectin, lepimectin, milbemycin

Juvenile hormone mimetics,

for example diofenolan, epofenonane, fenoxycarb, hydroprene, kinoprene, methoprene, pyriproxifen, triprene

Ecdysone agonists/disruptors

diacylhydrazines,

for example chromafenozide, halofenozide, methoxyfenozide, tebufenozide

Chitin biosynthesis inhibitors

benzoylureas,

for example bistrifluron, chlofluazuron, diflubenzuron, fluazuron, flucycloxuron, flufenoxuron, hexaflumuron, lufenuron, novaluron, noviflumuron, penfluron, teflubenzuron, triflumuron

buprofezin

cyromazine

Oxidative phosphorylation inhibitors, ATP disruptors

diafenthiuron

organotin compounds,

for example azocyclotin, cyhexatin, fenbutatin-oxide

Oxidative phosphorylation decouplers acting by interrupting the H-proton gradient

pyrroles,

for example chlorfenapyr

dinitrophenols,

for example binapacyrl, dinobuton, dinocap, DNOC, meptyldinocap

Site-I electron transport inhibitors

METIs,

for example fenazaquin, fenpyroximate, pyrimidifen, pyridaben, tebufenpyrad, tolfenpyrad

hydramethylnon

dicofol

Site-II electron transport inhibitors

cyenopyrafen, cyflumetofen, rotenone

Site-III electron transport inhibitors

Acequinocyl, fluacrypyrim

Microbial disruptors of the insect gut membrane

Bacillus thuringiensis strains

Lipid synthesis inhibitors

tetronic acids,

for example spirodiclofen, spiromesifen,

tetramic acids,

for example spirotetramat, cis-3-(2,5-dimethylphenyl)-4-hydroxy-8-methoxy-1-azaspiro[4.5]dec-3-en-2-one

Carboxamides,

for example flonicamid

Octopaminergic agonists,

for example amitraz

Inhibitors of magnesium-stimulated ATPase,

propargite

nereistoxin analogues,

for example thiocyclam hydrogen oxalate, thiosultap-sodium

Ryanodine receptor agonists,

benzenedicarboxamides,

for example flubendiamide

anthranilamides,

for example Rynaxypyr (3-bromo-N-{4-chloro-2-methyl-6-[(methylamino)carbonyl]phenyl}-1-(3-chloropyridin-2-yl)-1H-pyrazole-5-carboxamide)

Biologicals, hormones or pheromones

azadirachtin, Bacillus spec., Beauveria spec., Codlemone, Metarrhizium spec., Paecilomyces spec., Thuringiensin, Verticillium spec.

Active compounds with unknown or unspecific mechanisms of action
fumigants,
for example aluminium phosphide, methyl bromide, sulphuryl fluoride
antifeedants,
for example cryolite, flonicamid, pymetrozine
mite growth inhibitors,
for example clofentezine, etoxazole, hexythiazox
amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyridalyl, sulfluramid, tetradifon, tetrasul, triarathene, verbutin.

A mixture with other known active compounds, such as herbicides, fertilizers, growth regulators, safeners, semiochemicals, or else with agents for improving the plant properties, is also possible.

When used as insecticides, the active compounds according to the invention can furthermore be present in their commercially available formulations and in the use forms, prepared from these formulations, as a mixture with synergistic agents. Synergistic agents are compounds which increase the action of the active compounds, without it being necessary for the synergistic agent added to be active itself.

When used as insecticides, the active compounds according to the invention can furthermore be present in their commercially available formulations and in the use forms, prepared from these formulations, as a mixture with inhibitors which reduce degradation of the active compound after use in the environment of the plant, on the surface of parts of plants or in plant tissues.

The active compound content of the use forms prepared from the commercially available formulations can vary within wide limits. The active compound concentration of the use forms can be from 0.00000001 to 95% by weight of active compound, preferably between 0.00001 and 1% by weight.

The compounds are employed in a customary manner appropriate for the use forms.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on the surroundings, habitat or storage space by the customary treatment methods, for example by immersion, watering, spraying, evaporation, fogging, scattering, painting on, injection and, in the case of propagation material, in particular in the case of seeds, also by applying one or more coats.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts”, “parts of plants” and “plant parts” have been explained above.

Particularly preferably, plants of the plant cultivars which are in each case commercially available or in use are treated according to the invention. Plant cultivars are to be understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particular advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, sugar beet, tomatoes, peas and other vegetable varieties, cotton, tobacco, oilseed rape and also fruit plants (with the fruits apples, pears, citrus fruits and grapes), and particular emphasis is given to maize, soya beans, potatoes, cotton, tobacco, rice, canola and oilseed rape. Traits that are emphasized are in particular increased defence of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defence of plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLinkφ (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The plants listed can be treated according to the invention in a particularly advantageous manner with the compounds of the general formula I and/or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The active compounds according to the invention act not only against plant, hygiene and stored product pests, but also in the veterinary medicine sector against animal parasites (ecto- and endoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp., Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.

From the order of the Siphonapterida, for example, Pulex spp., Ctenocephalides spp., Xenopsylla spp., Ceratophyllus spp.

From the order of the heteropterida, for example, Cimex spp., Triatoma spp., Rhodnius spp., Panstrongylus spp.

From the order of the Blattarida, for example, Blatta orientalis, Periplaneta americana, Blattela germanica, Supella spp.

From the subclass of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reductions in productivity (for meat, milk, wool, hides, eggs, honey etc.) should be diminished, so that more economic and easier animal husbandry is possible by use of the active compounds according to the invention.

The active compounds according to the invention are used in the veterinary sector and in animal husbandry in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100- to 10000-fold dilution, or they can be used as a chemical bath.

It has furthermore been found that the compounds according to the invention also have a strong insecticidal action against insects which destroy industrial materials.

The following insects may be mentioned as examples and as preferred—but without any limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, heterobostrychus brunneus, Sinoxylon spec. Dinoderus minutus;

Hymenopterons, such as Sirex juvencus, Urocerus gigas, Urocerus gigas taignus, Urocerus augur;

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;

Bristletails, such as Lepisma saccharina.

Industrial materials in the present connection are to be understood as meaning non-living materials, such as, preferably, plastics, adhesives, sizes, papers and cardboards, leather, wood and processed wood products and coating compositions.

The ready-to-use compositions may, if appropriate, comprise further insecticides and, if appropriate, one or more fungicides.

With respect to possible additional additives, reference may be made to the insecticides and fungicides mentioned above.

The compounds according to the invention can likewise be employed for protecting objects which come into contact with saltwater or brackish water, in particular hulls, screens, nets, buildings, moorings and signalling systems, against fouling.

Furthermore, the compounds according to the invention, alone or in combinations with other active compounds, may be employed as antifouling agents.

In domestic, hygiene and stored-product protection, the active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Omithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp. From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Pemphigus spp., Phylloera vastatrix, Phthirus pubis.

From the order of the heteroptera, for example, Cimex hemipterus, Cimex lectularius, Rhodinus prolixus, Triatoma infestans.

In the field of household insecticides, they are used alone or in combination with other suitable active compounds, such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The compounds of the formula I-3 listed in the table below are known from WO 05/056556, WO 05/082907 and the international patent application having the application number PCT/EP 2006/001064, or they can be prepared by the processes described therein.

TABLE

| | | | | | (I-3) |
|---|---|---|---|---|---|
| 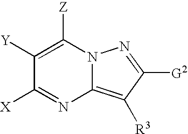 | | | | | |
| Ex. No. | X | Y | Z | R³ | G² |
| I-3-1 | Cl | 2-Cl, 4-F-Ph | NH—(R)—CHCH₃—C(CH₃)₃ | CO—NH—SO₂—CH₃ | H |
| I-3-2 | Cl | 2,4-Cl₂-Ph | NH—(R)—CH(CH₃)(CF₃) | CO—NH—SO₂—CH₃ | H |
| I-3-3 | Cl | 2-Cl, 4-F-Ph | NH—(R)—CHCH₃—C(CH₃)₃ | CO—NH—SO₂—CF₃ | H |
| I-3-4 | Cl | 2,4-Cl₂-Ph | NH—(R)—CHCH₃—C(CH₃)₃ | CO—NH—SO₂—CF₃ | H |
| I-3-5 | Cl | 2,4,6-F₃-Ph | NH—(R)—CHCH₃—CH(CH₃)₂ | CO—NH—SO₂—CH₃ | H |
| I-3-6 | Cl | 2,6-F₂, 4-Cl-Ph | NH—(R)—CHCH₃—C(CH₃)₃ | CONH—SO₂—CH₃ | H |
| I-3-7 | Cl | 2,4,6-F₃-Ph | NH—CH₂—CH(CH₃)₂ | CONH—SO₂—CH₃ | H |
| I-3-8 | Cl | 2,6-F₂, 4-Cl-Ph | NH—(R)—CHCH₃—CH(CH₃)₂ | CO—NH—SO₂—CH₃ | H |
| I-3-9 | Cl | 2,6-F₂, 4-Cl-Ph | NH—CH₂—CH(CH₃)₂ | CO—NH—SO₂—CH₃ | H |
| I-3-10 | Cl | 2,6-F₂, 4-Cl-Ph | NH—CH₂—C(CH₃)₃ | CO—NH—SO₂—CH₃ | H |
| I-3-11 | Cl | 2-Cl-Ph | NH—(R)—CHCH₃—CH(CH₃)₂ | CO—NH—SO₂—CH₃ | H |
| I-3-12 | Cl | 2-Cl-Ph | NH—(R)—CHCH₃—C(CH₃)₃ | CO—NH—SO₂—CH₃ | H |
| I-3-13 | Cl | 2-Cl, 6-F-Ph | NH—(R)—CHCH₃—CH(CH₃)₂ | CO—NH—SO₂—CH₃ | H |
| I-3-14 | Cl | 2-Cl, 6-F-Ph | NH—(R)—CHCH₃—C(CH₃)₃ | CO—NH—SO₂—CH₃ | H |
| I-3-15 | Cl | 2-Cl, 6-F-Ph | N—(CH₂)₂—CHCH₃—(CH₂)₂— | CO—NH—SO₂—CH₃ | H |
| I-3-16 | Cl | 2-Cl, 4,6-F₂-Ph | NH—(R)—CHCH₃—CH(CH₃)₂ | CO—NH—SO₂—CH₃ | H |
| I-3-17 | Cl | 2-Cl, 4,6-F₂-Ph | N—(CH₂)₂—CHCH₃—(CH₂)₂— | CO—NH—SO₂—CH₃ | H |
| I-3-18 | Cl | 2,4-Cl₂-Ph | NH—CH₂—C(CH₃)₃ | CO—NH—SO₂—CH₃ | H |
| I-3-19 | Cl | 2-Cl-Ph | NH—CH₂—C(CH₃)₃ | CO—NH—SO₂—CH₃ | H |
| I-3-20 | Cl | 2-Cl, 4,6-F₂-Ph | NH—(R)—CH(CF₃)(CH₃) | CO—NH—SO₂—CH₃ | H |
| I-3-21 | Cl | 2-Cl, 2,6-F₂-Ph | NH—CH(CH₃)₂ | CO—NH—SO₂—CH₃ | H |
| I-3-22 | Cl | 2-Cl, 6-F-Ph | NH—CH₂—C(CH₃)₃ | CO—NH—SO₂—CH₃ | H |
| I-3-23 | Cl | 2-Cl, 4-F-Ph | NH—CH₂—C(CH₃)₃ | CO—NH—SO₂—CH₃ | H |
| I-3-24 | Cl | 2,4-Cl₂-Ph | NH—(R)—CHCH₃—CH(CH₃)₂ | CO—NH—SO₂—CH₃ | H |
| I-3-25 | Cl | 2,4-Cl₂-Ph | NH—(R)—CHCH₃—C(CH₃)₃ | CO—NH—SO₂—CH₃ | H |
| I-3-26 | Cl | 2,6-F₂-4-CH₃-Ph | NH—(R)—CHCH₃—CH(CH₃)₂ | CO—NH—SO₂—CH₃ | H |
| I-3-27 | Cl | 2,4-Cl₂-Ph | NH—(R)—CHCH₃—CH(CH₃)₂ | CONH—SO₂C₂H₅ | H |
| I-3-28 | Cl | 2,4,6-F₃-Ph | NH—(R)—CHCH₃—C(CH₃)₃ | CO—NH—SO₂—CH₃ | H |
| I-3-29 | Cl | 2,4-Cl₂-Ph | NH—CH(CH₃)₂ | CO—NH—SO₂—CH₃ | H |
| I-3-30 | Cl | 2-Cl, 4-F-Ph | NH—CH(CH₃)₂ | CO—NH—SO₂—CH₃ | H |
| I-3-31 | Cl | 2-Cl-Ph | NH—CH(CH₃)₂ | CO—NH—SO₂—CH₃ | H |
| I-3-32 | Cl | 2,4,6-F₃-Ph | NH—CH(CH₃)₂ | CO—NH—SO₂—CH₃ | H |
| I-3-33 | Cl | 2,6-F₂-4-CH₃-Ph | NH—(R)—CHCH₃—C(CH₃)₃ | CO—NH—SO₂—CH₃ | H |
| I-3-35 | Cl | 2,4-F₂-Ph | NH—(R)—CHCH₃—CH(CH₃)₂ | CO—NH—SO₂—CH₃ | H |
| I-3-36 | Cl | 2-Cl-4-CH₃-Ph | NH—(R)—CHCH₃—C(CH₃)₃ | CO—NH—SO₂—CH₃ | H |
| I-3-37 | Cl | 2,4,5-F₃-Ph | NH—(R)—CHCH₃—C(CH₃)₃ | CO—NH—SO₂—CH₃ | H |
| I-3-38 | Cl | 2-Cl-Ph | NH—(R)—CHCH₃—C(CH₃)₃ | CO—NH—SO₂—CH₃ | H |
| I-3-39 | Cl | 2,4,6-F₃-Ph | 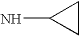 | CO—NH—SO₂—CH₃ | H |
| I-3-40 | Cl | 2,4,6-F₃-Ph | NH—CHCH₃-4-Cl-Ph | CO—NH—SO₂—CH₃ | H |
| I-3-41 | Cl | Ph | NH—CHCH₃—C(CH₃)₃ | 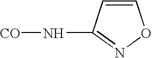 | H |
| I-3-42 | Cl | 2,4,6-F₃-Ph | NH—(S)—CH—(CF₃)—CH₃ | 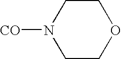 | H |
| I-3-43 | Cl | 2,4,6-F₃-Ph | NH—(S)—CH—(CF₃)—CH₃ | CO—N(CH₃)₂ | H |
| I-3-44 | Cl | 2,4,6-F₃-Ph | NH—CH₂—CH(CH₃)₂ | CO—O—CHCH₃—CO₂CH₃ | H |
| I-3-45 | Cl | 4-Cl, 2,6-F₂-Ph | NH—(R)—CHCH₃—CH(CH₃)₂ | 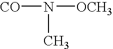 | H |
| I-3-46 | Cl | 2-Cl, 4,6-F₂-Ph | NH—(R)—CHCH₃—CH(CH₃)₂ | 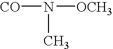 | H |
| I-3-47 | Cl | 2-Cl, 4,6-F₂-Ph | NH—(S)—CHCF₃CH₃ | 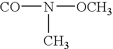 | H |
| I-3-48 | Cl | 2,4,5-F₃-Ph | NH—CHCH₃—C(CH₃)₃ | 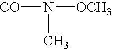 | H |
| I-3-49 | Cl | 2-Cl, 4-F-Ph | NH—(R)—CHCH₃—CH(CH₃)₂ | CO₂—(CH₂)₂—OCH₃ | H |
| I-3-50 | Cl | 2,4,6-F₃-Ph | NH—(R)—CHCH₃—C(CH₃)₃ | CO₂CH₃ | H |
| I-3-51 | Cl | 2,4,6-F₃-Ph | NH—(R)—CHCH₃—CH(CH₃)₂ | CO₂CH₃ | H |
| I-3-52 | Cl | 2,4,6-F₃-Ph | NH—(S)—CH—(CF₃)—CH₃ | CO₂CH₃ | H |
| I-3-53 | Cl | 2-Cl, 4-F-Ph | NH—(R)—CHCH₃—C(CH₃)₃ | CO₂—C₂H₅ | H |
| I-3-54 | Cl | 2-Cl, 6-F-Ph | NH—(S)—CH—(CF₃)—CH₃ | CO₂—(CH₂)₂—OCH₃ | H |
| I-3-55 | Cl | 2-Cl, 6-F-Ph | NH—(S)—CH—(CF₃)—CH₃ | CO₂—CH₂—CF₃ | H |
| I-3-56 | Cl | 2,4,6-F₃-Ph | NH—(R)—CHCH₃—C(CH₃)₃ | 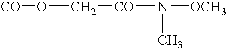 | H |
| I-3-57 | Cl | 2-Cl, 4,6-F₂-Ph | NH—(R)—CHCH₃—CH(CH₃)₂ | CO₂CH₃ | H |
| I-3-58 | Cl | 2-Cl, 4,6-F₂-Ph | NH—(R)—CHCH₃—C(CH₃)₃ | CO₂CH₃ | H |
| I-3-59 | Cl | 2,4,6-F₃-Ph | NH—(R)—CHCH₃—CH(CH₃)₂ | CO—(O—CH₂—CH₂)₂—OCH₃ | H |
| I-3-60 | Cl | 2,4,6-F₃-Ph | NH—(R)—CHCH₃—CH(CH₃)₂ | CO—CHCH₃—CO₂CH₃ | H |
| I-3-61 | Cl | 2-Cl, 4,6-F₂-Ph | NH—(R)—CHCH₃—CH(CH₃)₂ | 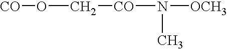 | H |
| I-3-62 | Cl | 2-Cl, 4,6-F₂-Ph | NH—(R)—CHCH₃—C(CH₃)₃ | 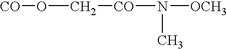 | H |
| I-3-63 | Cl | 2,4,6-F₃-Ph | NH—(R)—CHCH₃—CH(CH₃)₂ | CO—O—CH₂—CO₂CH₃ | H |
| I-3-64 | Cl | 2,4,6-F₃-Ph | NH—(R)—CHCH₃—CH(CH₃)₂ | CO—(O—CH₂—CH₂)₂—OCH₃ | H |
| I-3-65 | Cl | 2,4,6-F₃-Ph | NH—(R)—CHCH₃—CH(CH₃)₂ | CO—O—CHCH₃—CO₂CH₃ | H |
| I-3-66 | Cl | 2,4,6-F₃-Ph | NH—(R)—CHCH₃—CH(CH₃)₂ | CO—O—(CH₂)₂—OCH₃ | H |
| I-3-67 | Cl | 2,4,6-F₃-Ph | NH—(R)—CHCH₃—C(CH₃)₃ | CO—O—CHCH₃—CO₂CH₃ | H |
| I-3-68 | Cl | 2,4,6-F₃-Ph | NH—(R)—CHCH₃—C(CH₃)₃ | CO—(O—CH₂—CH₂)₂—OCH₃ | H |
| I-3-69 | Cl | 2,4,6-F₃-Ph | NH—(S)—CHCH₃—C(CH₃)₃ | CO—O—(CH₂)₂—OCH₃ | H |
| I-3-70 | Cl | 4-Cl, 2,6-F₂-Ph | NH—(R)—CHCH₃—C(CH₃)₃ | CO—O—CHCH₃—CO₂CH₃ | H |
| I-3-71 | Cl | 4-Cl, 2,6-F₂-Ph | NH—(R)—CHCH₃—C(CH₃)₃ | CO—(O—CH₂—CH₂)₂—OCH₃ | H |
| I-3-72 | Cl | 4-Cl, 2,6-F₂-Ph | NH—(R)—CHCH₃—C(CH₃)₃ | CO—O—CH₂—CO₂CH₃ | H |
| I-3-73 | Cl | 4-Cl, 2,6-F₂-Ph | NH—(R)—CHCH₃—C(CH₃)₃ | CO—O—CHCH₃CO—NHCH₃ | H |
| I-3-74 | Cl | 2,4,6-F₃-Ph | NH—(R)—CH₂—CH(CH₃)₂ | CO—O—CHCH₃—CO₂CH₃ | H |
| I-3-75 | Cl | 2,4,6-F₃-Ph | NH—(R)—CHCH₃—CH(CH₃)₂ | CO—O—CH₂—CO₂CH₃ | H |
| I-3-76 | Cl | 4-Cl, 2,6-F₂-Ph | NH—(R)—CHCH₃—C(CH₃)₃ | CO—(CH₂)₂—OCH₃ | H |
| I-3-77 | Cl | 2,4,6-F₃-Ph | NH—CH₂—CH(CH₃)₂ | CO—O—CHCH₃—CO₂CH₃ | H |
| I-3-78 | Cl | 4-Cl, 2,6-F₂-Ph | NH—(R)—CHCH₃—CH(CH₃)₂ | CO—O—CHCH₃—CO₂CH₃ | H |
| I-3-79 | Cl | 4-Cl, 2,6-F₂-Ph | NH—(R)—CHCH₃—CH(CH₃)₂ | CO—(O—CH₂—CH₂)₂—OCH₃ | H |
| I-3-80 | Cl | 4-Cl, 2,6-F₂-Ph | NH—(R)—CHCH₃—CH(CH₃)₂ | CO—O—CH₂—CO₂CH₃ | H |
| I-3-81 | Cl | 4-Cl, 2,6-F₂-Ph | NH—(R)—CHCH₃—CH(CH₃)₂ | CO—O—(CH₂)₂—OCH₃ | H |
| I-3-82 | Cl | 2,4-Cl₂-Ph | NH—CH(CH₃)₂ | CO—(O—CH₂—CH₂)₂—OCH₃ | H |
| I-3-83 | Cl | 2,4,6-F₃-Ph | NH—CH(CH₃)₂ | CO—O—CH₂—CO₂CH₃ | H |
| I-3-84 | Cl | 2-Cl, 4,6-F₂-Ph | NH—(S)—CH(CF₃)(CH₃) | CO—O—CHCH₃—CO₂CH₃ | H |
| I-3-85 | Cl | 4-Cl, 2,6-F₂-Ph | NH—CH(CH₃)₂ | CO—O—CHCH₃—CO₂CH₃ | H |
| I-3-86 | Cl | 2-Cl, 4,6-F₂-Ph | NH—(S)—CH(CF₃)(CH₃) | CO—(O—CH₂—CH₂)₂—OCH₃ | H |
| I-3-87 | Cl | 2-Cl, 4,6-F₂-Ph | NH—(S)—CH(CF₃)(CH₃) | CO—O—CH₂—CO₂CH₃ | H |
| I-3-88 | Cl | 2-Cl, 4,6-F₂-Ph | NH—(R)—CHCH₃—C(CH₃)₃ | CO—O—(CH₂)₂—OCH₃ | H |
| I-3-89 | Cl | 2-Cl, 4,6-F₂-Ph | NH—(S)—CH(CF₃)(CH₃) | CO—O—(CH₂)₂—OCH₃ | H |
| I-3-90 | Cl | 4-Cl, 2,6-F₂-Ph | NH—CH(CH₃)₂ | CO—O—(CH₂)₂—OCH₃ | H |
| I-3-91 | Cl | 4-CN, 2-Cl-Ph | NH—(S)—CH(CF₃)(CH₃) | CO₂CH₃ | H |
| I-3-92 | Cl | 2-Cl, 4-F-Ph | NH—(R)—CHCH₃—C(CH₃)₃ | 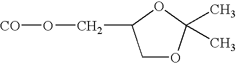 | H |
| I-3-93 | Cl | 2,4,5-F₃-Ph | NH—(R)—CHCH₃—C(CH₃)₃ | CO—O—CH₂—CO₂—CH₃ | H |
| I-3-94 | Cl | 4-Cl, 2,6-F₂-Ph | NH—CH(CH₃)₂ | CO—O—CH₂—CO₂CH₃ | H |
| I-3-95 | Cl | 2,4,5-F₃-Ph | NH—(R)—CHCH₃—C(CH₃)₃ | CO—(O—CH₂—CH₂)₂—OCH₃ | H |
| I-3-96 | Cl | 2,4,5-F₃-Ph | NH—(R)—CHCH₃—C(CH₃)₃ | CO₂CH₃ | H |
| I-3-97 | Cl | 2,5-F₂-Ph | NH—(S)—CH(CF₃)(CH₃) | CO₂CH₃ | H |
| I-3-98 | Cl | 3,4-O—CH₂—O-Ph | NH—CH(CH₃)₂ | CO₂CH₃ | H |
| I-3-99 | Cl | 2,5-F₂-Ph | NH—(R)—CHCH₃—C(CH₃)₃ | 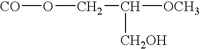 | H |
| I-3-100 | Cl | 2,5-F₂-Ph | NH—(R)—CHCH₃—C(CH₃)₃ | CO—O—(CH₂)₂—N(C₂H₅)₂ | H |
| I-3-101 | Cl | 2,5-F₂-Ph | NH—(R)—CHCH₃—C(CH₃)₃ | 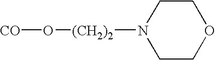 | H |
| I-3-102 | Cl | 2-Br, 4-F-Ph | NH—(R)—CHCH₃—C(CH₃)₃ | CO₂CH₃ | H |
| I-3-103 | Cl | 2,5-F₂-Ph | NH—(R)—CHCH₃—C(CH₃)₃ | CO—O—(CH₂)₂—N(C₂H₅)₂ | H |
| I-3-104 | Cl | 2,5-F₂-Ph | NH—(R)—CHCH₃—C(CH₃)₃ | 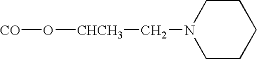 | H |
| I-3-105 | Cl | 2-Br, 5-F-Ph | NH—(R)—CHCH₃—C(CH₃)₃ | CO—O—CHCH₂F—CH₂—OCH₃ | H |
| I-3-106 | Cl | 2-Br, 4-F-Ph | NH—(R)—CHCH₃—C(CH₃)₃ | 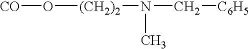 | H |
| I-3-107 | Cl | 2-Br, 4-F-Ph | NH—(R)—CHCH₃—C(CH₃)₃ | 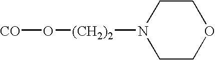 | H |
| I-3-108 | Cl | 2-Br, 4-F-Ph | NH—(R)—CHCH₃—C(CH₃)₃ | CO—O—(CH₂)₃—N(C₂H₅)₂ | H |
| I-3-109 | Cl | 2,4-F₂-Ph | NH—(R)—CHCH₃—CH(CH₃)₂ | CO—O—(CH₂)₂—O—(CH₂)₂—N(CH₃)₂ | H |
| I-3-110 | Cl | 2-Br-Ph | NH—CH(CH₃)₂ | CO₂CH₃ | H |
| I-3-111 | Cl | 2,4,6-F₃-Ph | NH—CH(CH₃)₂ | CO₂CH₃ | H |
| I-3-112 | Cl | 2-Cl, 4-F-Ph | NH—(R)—CHCH₃—CH(CH₃)₂ | CO₂H | H |
| I-3-113 | Cl | 2-Cl-Ph | NH—(R)—CHCH₃—CH(CH₃)₂ | CO₂H | H |
| I-3-114 | Cl | 2,4,6-F₃-Ph | NH—(R)—CHCH₃—C(CH₃)₃ | CO₂H | H |
| I-3-115 | Cl | 2,4,6-F₃-Ph | NH—(S)—CH(CF₃)—(CH₃) | CO₂H | H |
| I-3-116 | Cl | 2,4,6-F₃-Ph | NH—(R)—CHCH₃—C(CH₃)₃ | CO₂⁻ K⁺ | H |
| I-3-117 | Cl | 2,4,6-F₃-Ph | NH—(R)—CHCH₃—CH(CH₃)₂ | CO₂⁻ K⁺ | H |
| I-3-118 | Cl | 2,4,6-F₃-Ph | NH—(R)—CHCH₃—CH(CH₃)₂ | CO₂H | H |
| I-3-119 | Cl | 2-Cl, 4,6-F₂-Ph | NH—(R)—CHCH₃—CH(CH₃)₂ | CO₂H | H |
| I-3-120 | Cl | 2-Cl, 4,6-F₂-Ph | NH—(R)—CHCH₃—C(CH₃)₃ | CO₂H | H |
| I-3-121 | Cl | 2,4-Cl₂-Ph | NH—(R)—CHCH₃—CH(CH₃)₂ | CO₂H | H |
| I-3-122 | Cl | 4-Cl, 2,6-F₂-Ph | NH—(R)—CHCH₃—C(CH₃)₃ | CO₂H | H |
| I-3-123 | Cl | 2-CH₃, 4-F-Ph | NH—(R)—CHCH₃—CH(CH₃)₂ | CO₂⁻ K⁺ | H |
| I-3-124 | Cl | 2-Cl, 4,6-F₂-Ph | NH—(S)—CH(CF₃)—(CH₃) | CO₂H | H |
| I-3-125 | Cl | 2,5-F₂-Ph | NH—(R)—CHCH₃—C(CH₃)₃ | CO₂H | H |
| I-3-126 | Cl | 2,5-F₂-Ph | NH—(R)—CHCH₃—C(CH₃)₃ | CO₂⁻ Cs⁺ | H |
| I-3-127 | Cl | 2,4,5-F₃-Ph | NH—(R)—CHCH₃—C(CH₃)₃ | CO₂H | H |
| I-3-128 | Cl | 2,4,5-F₃-Ph | NH—(R)—CHCH₃—CH(CH₃)₂ | CO₂H | H |
| I-3-129 | Cl | 4-Cl, 2-F-Ph | NH—(R)—CHCH₃—C(CH₃)₃ | CO₂⁻ Li⁺ | H |
| I-3-130 | Cl | 4-Cl, 2-F-Ph | NH—(R)—CHCH₃—C(CH₃)₃ | CO₂⁻ Li⁺ | H |
| I-3-131 | Cl | 4-Cl, 2-F-Ph | NH—(R)—CHCH₃—CH(CH₃)₂ | CO₂⁻ K⁺ | H |
| I-3-132 | Cl | 4-Cl, 2-F-Ph | NH—(R)—CHCH₃—CH(CH₃)₂ | CO₂⁻ NH₄⁺ | H |
| I-3-133 | Cl | 4-Cl, 2-F-Ph | NH—(R)—CHCH₃—CH(CH₃)₂ | CO₂⁻ NH₂(CH(CH₃)₂)₂⁺ | H |
| I-3-134 | Cl | 4-Cl, 2-F-Ph | NH—(R)—CHCH₃—CH(CH₃)₂ | CO₂⁻ 1/2 Ca²⁺ | H |
| I-3-135 | Cl | 4-Cl, 2-F-Ph | NH—(R)—CHCH₃—CH(CH₃)₂ | CO₂⁻ NH₃—CH₂—CH(CH₃)₂⁺ | H |
| I-3-136 | Cl | 4-Cl, 2-F-Ph | NH—(R)—CHCH₃—CH(CH₃)₂ | CO₂⁻ NH₃—C₄H₉⁺ | H |
| I-3-137 | Cl | 4-Cl, 2-F-Ph | NH—(R)—CHCH₃—CH(CH₃)₂ | CO₂⁻ NH₃—CH(CH₃)₂⁺ | H |
| I-3-138 | Cl | 4-Cl, 2-F-Ph | NH—(R)—CHCH₃—CH(CH₃)₂ | CO₂⁻ NH₃—C₃H₇⁺ | H |
| I-3-139 | Cl | 4-Cl, 2-F-Ph | NH—(R)—CHCH₃—CH(CH₃)₂ | 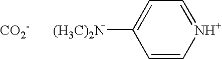 | H |
| I-3-140 | Cl | 2-Br, 4-F-Ph | NH—(R)—CHCH₃—CH(CH₃)₂ | CO₂⁻ NH(C₂H₅)₃⁺ | H |
| I-3-141 | Cl | 2-Br, 4-F-Ph | NH—(R)—CHCH₃—CH(CH₃)₂ | 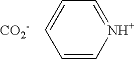 | H |
| I-3-142 | Cl | 2-Br, 4-F-Ph | NH—(R)—CHCH₃—CH(CH₃)₂ | 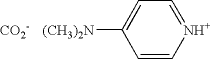 | H |
| I-3-143 | Cl | 2-Br, 4-F-Ph | NH—(R)—CHCH₃—CH(CH₃)₂ | CO₂⁻ Na⁺ | H |
| I-3-144 | Cl | 2-Br, 4-F-Ph | NH—(R)—CHCH₃—CH(CH₃)₂ | CO₂⁻ K⁺ | H |
| I-3-145 | Cl | 2-Br, 4-F-Ph | NH—(R)—CHCH₃—CH(CH₃)₂ | CO₂⁻ NH₄⁺ | H |
| I-3-146 | Cl | 2-Br, 4-F-Ph | NH—(R)—CHCH₃—CH(CH₃)₂ | CO₂⁻ Li⁺ | H |
| I-3-147 | Cl | 2-Br, 4-F-Ph | NH—(R)—CHCH₃—CH(CH₃)₂ | CO₂ ^{1/2} Mg²⁺ | H |
| I-3-148 | Cl | 2-Br, 4-F-Ph | NH—(R)—CHCH₃—CH(CH₃)₂ | CO₂⁻ Cs⁺ | H |
| I-3-149 | Cl | 2-Br, 4-F-Ph | NH—(R)—CHCH₃—C(CH₃)₃ | CO₂⁻ K⁺ | H |
| I-3-150 | Cl | 2-Br, 4-F-Ph | NH—(R)—CHCH₃—C(CH₃)₃ | CO₂⁻ NH₄⁺ | H |
| I-3-151 | Cl | 2-Cl, 5-F-Ph | NH—(R)—CHCH₃—CH(CH₃)₂ | 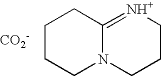 | H |
| I-3-152 | Cl | 2-Cl, 5-F-Ph | NH—(R)—CHCH₃—CH(CH₃)₂ | CO₂⁻ NH₂(CH(CH₃)₂)₂⁺ | H |
| I-3-153 | Cl | 2-Cl, 5-F-Ph | NH—(R)—CHCH₃—CH(CH₃)₂ | CO₂⁻ NH₂(C₂H₅)₂⁺ | H |
| I-3-154 | Cl | 2,5-F₂-Ph | NH—(R)—CHCH₃—C(CH₃)₃ | CO₂^{- 1/2} Zn²⁺ | H |
| I-3-155 | Cl | 2-Cl, 5-F-Ph | NH—(R)—CHCH₃—CH(CH₃)₂ | CO₂⁻ Cs⁺ | H |
| I-3-156 | Cl | 2-Cl, 5-F-Ph | NH—(R)—CHCH₃—CH(CH₃)₂ | CO₂^{- 1/2} Mg²⁺ | H |
| I-3-157 | Cl | 2,5-F₂-Ph | NH—(R)—CHCH₃—C(CH₃)₃ | CO₂^{- 1/2} Fe²⁺ | H |
| I-3-158 | Cl | 2,5-F₂-Ph | NH—(R)—CHCH₃—C(CH₃)₃ | CO₂^{- 1/3} Fe³⁺ | H |
| I-3-159 | Cl | 2,5-F₂-Ph | NH—(R)—CHCH₃—C(CH₃)₃ | CO₂^{- 1/2} Mg²⁺ | H |
| I-3-160 | Cl | 2,5-F₂-Ph | NH—(R)—CHCH₃—CH(CH₃)₂ | CO₂⁻ Cs⁺ | H |
| I-3-161 | Cl | 2,5-F₂-Ph | NH—(R)—CHCH₃—C(CH₃)₃ | CO₂^{-1/2} Cu²⁺ | H |
| I-3-162 | Cl | 2,5-F₂-Ph | NH—(S)—CHCH₃—CH(CH₃)₂ | CO₂^{- 1/2} Cu²⁺ | H |
| I-3-163 | Cl | 2-CH₃-Ph | 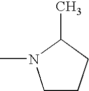 | CO₂H | H |
| I-3-164 | Cl | 2-Cl, 4-F-Ph | NH—(R)—CHCH₃—CH(CH₃)₂ | 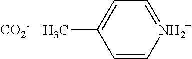 | H |
| I-3-165 | Cl | 2-Cl, 4-F-Ph | NH—(R)—CHCH₃—CH(CH₃)₂ | 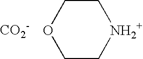 | H |
| I-3-166 | Cl | 2-Cl, 4-F-Ph | NH—(R)—CHCH₃—CH(CH₃)₂ | 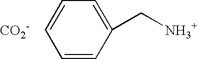 | H |
| I-3-167 | Cl | 2-Cl-Thienyl | NH—(R)—CHCH₃—C(CH₃)₃ | CO₂⁻ Na⁺ | H |
| I-3-168 | Cl | 2-F-Ph | NH—(R)—CHCH₃—CH(CH₃)₂ | CO₂⁻ Na⁺ | H |
| I-3-169 | Cl | 2-Br-Ph | NH—(R)—CHCH₃—CH(CH₃)₂ | CO₂H | H |
| I-3-170 | Cl | 2-Br-Ph | 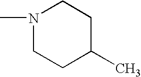 | CO₂H | H |
| I-3-171 | Cl | 2-Cl, 4-CH₃-Ph | NH—(R)—CHCH₃—C(CH₃)₃ | CO₂⁻ Na⁺ | H |
| I-3-172 | Cl | 2-Cl, 4-CH₃-Ph | NH—CHCH₃—C(CH₃)₃ | CO₂⁻K⁺ | H |
| I-3-173 | Cl | 2,4,6-F₃-Ph | NH—(S)—CH(CF₃)(CH₃) | CO₂H | H |
| I-3-174 | Cl | 2,4,6-F₃-Ph | NH—CH(CH₃)₂ | CO₂H | H |
| I-3-175 | Cl | 2,4,6-F₃-Ph | 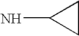 | CO₂H | H |
| I-3-176 | Cl | 2,4,6-F₃-Ph | NH—(R)—CHCH₃—C(CH₃)₃ | CO₂⁻ Na⁺ | H |
| I-3-177 | Cl | 2-Cl, 6-F-Ph | NH—(R)—CHCH₃—CH(CH₃)₂ | CO₂H | H |
| I-3-178 | Cl | 2-Cl, 6-F-Ph | NH—(R)—CHCH₃—C(CH₃)₃ | CO₂H | H |
| I-3-179 | Cl | 2,6-F₂, 4-CH₃-Ph | NH—(R)—CHCH₃—CH(CH₃)₂ | CO₂H | H |
| I-3-180 | Cl | 2,6-F₂, 4-CH₃-Ph | NH—(R)—CHCH₃—C(CH₃)₃ | CO₂H | H |
| I-3-181 | Cl | 2,4-Cl₂, 5-F-Ph | NH—(R)—CHCH₃—CH(CH₃)₂ | CO₂H | H |
| I-3-182 | Cl | 2,4-Cl₂, 5-F-Ph | NH—(R)—CHCH₃—C(CH₃)₃ | CO₂H | H |
| I-3-183 | Cl | 2,4,5-Cl₃-Ph | NH—(R)—CHCH₃—C(CH₃)₃ | CO₂H | H |
| I-3-184 | Cl | 2,4,5-F₃-Ph | NH—(S)—CH—(CF₃)—CH₃ | CO₂H | H |
| I-3-185 | Cl | 2-F, 4-CH₃-Ph | NH—(R)—CHCH₃—CH(CH₃)₂ | CO₂H | H |
| I-3-186 | Cl | 2-F, 4-CH₃-Ph | NH—(R)—CHCH₃—C(CH₃)₃ | CO₂H | H |
| I-3-187 | Cl | 2,5-F₂, 4-CH₃-Ph | NH—(R)—CHCH₃—CH(CH₃)₂ | CO₂H | H |
| I-3-188 | Cl | 2,5-F₂, 4-CH₃-Ph | NH—(R)—CHCH₃—C(CH₃)₃ | CO₂H | H |
| I-3-189 | Cl | 2,5-F₂, 4-CH₃-Ph | NH—CHCH₃—CH₂CH₃ | CO₂H | H |
| I-3-190 | Cl | 2-Cl-Ph | NH—(R)—CHCH₃—CH(CH₃)₂ | CO₂H | Cl |
| I-3-191 | Cl | 2-Br, 4-F-Ph | NH—(R)—CHCH₃—C(CH₃)₃ | CO₂H | H |
| I-3-192 | Cl | 2-Br, 4-F-Ph | NH—(R)—CHCH₃—CH(CH₃)₂ | CO₂H | H |
| I-3-193 | Cl | 2,5-F₂-Ph | NH—(R)—CHCH₃—CH₂CH₃ | CO₂H | H |
| I-3-194 | Cl | 2,5-F₂-Ph | 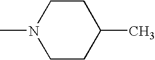 | CO₂H | H |
| I-3-195 | Cl | 2,4-F₂-Ph | NH—(R)—CHCH₃—C(CH₃)₃ | CO₂H | H |
| I-3-196 | Cl | 2,4-F₂-Ph | 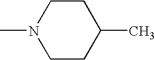 | CO₂H | H |
| I-3-197 | Cl | 2,4-F₂-Ph | NH—CH₂—CH(CH₃)₂ | CO₂H | H |
| I-3-198 | Cl | 2-Cl, 4-CH₃-Ph | NH—(R)—CHCH₃—C(CH₃)₃ | CO₂H | H |
| I-3-199 | Cl | 2-F-Ph | NH—(R)—CHCH₃—C(CH₃)₃ | CO₂H | H |
| I-3-200 | Cl | 2-F-Ph | NH—(R)—CHCH₃—CH(CH₃)₂ | CO₂H | H |
| I-3-201 | Cl | 2-Cl, 6-F-Ph | NH—CH(CH₃)₂ | CO₂H | H |
| I-3-202 | Cl | 2-Cl, 4-F-Ph | NH—(R)—CHCH₃—C(CH₃)₃ | CO₂H | H |
| I-3-203 | Cl | 2-Cl, 4-F-Ph | NH—(S)—CH—(CF₃)—CH₃ | CO₂H | H |
| I-3-204 | Cl | 2,4-Cl₂-Ph | NH—(S)—CH—(CF₃)—CH₃ | CO₂H | H |
| I-3-205 | Cl | 2-Cl, 4,6-F₂-Ph | 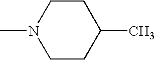 | CO₂H | H |
| I-3-206 | Cl | 2,4-Cl₂-Ph | NH—(R)—CHCH₃—C(CH₃)₃ | CO₂H | H |
| I-3-207 | Cl | 2,4,6-F₃-Ph | NH—CH₂—CH(CH₃)₂ | CO₂H | H |
| I-3-208 | Cl | 2,4,6-F₃-Ph | NH—CH₂—C(CH₃)₃ | CO₂H | H |
| I-3-209 | Cl | 2,4-Cl₂-Ph | NH—CH(CH₃)₂ | CO₂H | H |
| I-3-210 | Cl | 2,4-Cl₂-Ph | NH—CH₂—CH(CH₃)₂ | CO₂H | H |
| I-3-211 | Cl | 2-Cl, 6-F-Ph | NH—CH₂—C(CH₃)₃ | CO₂H | H |
| I-3-212 | Cl | 4-Cl, 2,6-F₂-Ph | NH—CH₂—CH(CH₃)₂ | CO₂H | H |
| I-3-213 | Cl | 4-Cl, 2,6-F₂-Ph | NH—CH₂—C(CH₃)₃ | CO₂H | H |
| I-3-214 | Cl | 2-Cl-Ph | NH—(R)—CHCH₃—C(CH₃)₃ | CO₂H | Cl |
| I-3-215 | Cl | 2,5-F₂-Ph | NH—(R)—CHCH₃—CH(CH₃)₂ | CO₂H | H |
| I-3-216 | Cl | 2-Cl, 5-F-Ph | NH—(R)—CHCH₃—C(CH₃)₃ | CO₂H | H |
| I-3-217 | Cl | 2-Cl, 5-F-Ph | NH—(R)—CHCH₃—CH(CH₃)₂ | CO₂H | H |
| I-3-218 | Cl | 4-Cl, 2-F-Ph | NH—(R)—CHCH₃—C(CH₃)₃ | CO₂H | H |
| I-3-219 | Cl | 4-Cl, 2-F-Ph | NH—CHCH₃—CH₂CH₃ | CO₂H | H |
| I-3-220 | Cl | 4-Cl, 2-F-Ph | NH—(R)—CHCH₃—CH(CH₃)₂ | CO₂H | H |
| I-3-221 | Cl | 2-Cl, 5-F-Ph | NH—CH₂—C(CH₃)₃ | CO₂H | H |
| I-3-222 | Cl | 3,4-O—CH₂—O-Ph | NH—(R)—CHCH₃—CH(CH₃)₂ | CO₂H | H |
| I-3-223 | Cl | 2,4-Cl₂, 5-CH₃-Ph | NH—(R)—CHCH₃—CH(CH₃)₂ | CO₂H | H |
| I-3-224 | Cl | 3,4-O—CH₂—O-Ph | NH—(R)—CHCH₃—C(CH₃)₃ | CO₂H | H |
| I-3-225 | Cl | 2,4-Cl₂, 5-CH₃-Ph | NH—(R)—CHCH₃—C(CH₃)₃ | CO₂H | H |
| I-3-226 | Cl | 5-Br, 2-Cl-Ph | NH—(R)—CHCH₃—C(CH₃)₃ | CO₂H | H |
| I-3-227 | Cl | 5-Br, 2-Cl-Ph | NH—(R)—CHCH₃—CH(CH₃)₂ | CO₂H | H |
| I-3-228 | Cl | 2,5-Cl₂-Ph | NH—(R)—CHCH₃—CH(CH₃)₂ | CO₂H | H |
| I-3-229 | Cl | 2,5-Cl₂-Ph | NH—(R)—CHCH₃—C(CH₃)₃ | CO₂H | H |
| I-3-230 | Cl | 4-Cl, 2-F-Ph | NH—(R)—CHCH₃—CH₂CH₃ | CO₂H | H |
| I-3-231 | Cl | 2-Cl, 5-CH₃O-Ph | NH—(R)—CHCH₃—C(CH₃)₃ | CO₂H | H |
| I-3-232 | Cl | 4-Cl, 2-F-Ph | N—(CH₃)—CH(CH₃)₂ | CO₂H | H |
| I-3-233 | Cl | 2,5-F₂-Ph | N—(CH₃)—CH(CH₃)₂ | CO₂H | H |
| I-3-234 | Cl | 2,6-F₂-Ph | NH—CHCH₃—C(CH₃)₃ | CO₂H | H |
| I-3-235 | Cl | 2,4-F₂-Ph | NH—(R)—CHCH₃—CH(CH₃)₂ | CO₂H | H |
| I-3-236 | Cl | 2-Cl, 4-CH₃-Ph | NH—(R)—CHCH₃—CH(CH₃)₂ | CO₂H | H |
| I-3-237 | Cl | 2-Cl, 4-CH₃-Ph | NH—(S)—CH—(CF₃)—CH₃ | CO₂H | H |
| I-3-238 | Cl | 2-Cl, 4-CH₃-Ph | NH—(S)—CHCH₃—C(CH₃)₃ | CO₂H | H |
| I-3-239 | Cl | 2-Cl, 4-CH₃-Ph | NH—CHCH₃—C(CH₃)₃ | CO₂H | H |
| I-3-240 | Cl | 2-Br-Ph | NH—CH(CH₃)₂ | CO₂H | H |
| I-3-241 | Cl | 2-CH₃-Ph | NH—CH(CH₃)₂ | CO₂H | H |
| I-3-242 | Cl | 2-Cl, 4-F-Ph | NH—CH(CH₃)₂ | CO₂H | H |
| I-3-243 | Cl | 2-Cl, 4-CH₃-Ph | NH—CH(CH₃)₂ | CO₂H | H |
| I-3-244 | Cl | 5-Br, 2-Cl-Ph | NH—CH(CH₃)₂ | CO₂H | H |
| I-3-245 | Cl | 2-Cl, 6-F-Ph | NH—(R)—CHCH₃—C(CH₃)₃ | CO₂CH₃ | H |
| I-3-246 | Cl | 2-Cl, 4-F-Ph | NH—(R)—CHCH₃—C(CH₃)₃ | CO—O—CH₂—CH═CH₂ | H |
| I-3-247 | Cl | 2-Cl, 4-F-Ph | NH—(R)—CHCH₃—C(CH₃)₃ | CO—(O—CH₂—CH₂)₃—OCH₃ | H |
| I-3-248 | Cl | 2,4-Cl₂-Ph | NH—(R)—CHCH₃—C(CH₃)₃ | CO—O—CHCH₃—CO₂CH₃ | H |
| I-3-249 | Cl | 2,4-Cl₂-Ph | NH—(R)—CHCH₃—C(CH₃)₃ | CO—(O—CH₂—CH₂)₃—OCH₃ | H |
| I-3-250 | Cl | 2,6-F₂, 4-CH₃-Ph | NH—(R)—CHCH₃—C(CH₃)₃ | CO₂CH₃ | H |
| I-3-251 | Cl | 2,6-F₂, 4-CH₃-Ph | NH—(R)—CHCH₃—C(CH₃)₃ | CO—O—CH₂—CO₂CH₃ | H |
| I-3-252 | Cl | 2,4,5-Cl₃-Ph | NH—(R)—CHCH₃—C(CH₃)₃ | CO₂CH₃ | H |
| I-3-253 | Cl | 2,4,5-F₃-Ph | NH—CH(CF₃)(CH₃) | CO₂(CH₂)₂OCH₃ | H |
| I-3-254 | Cl | 2,4,6-F₃-Ph | NH—CH(CH₃)₂ | CO—(O—CH₂—CH₂)₂—OCH₃ | H |
| I-3-255 | Cl | 2,4-Cl₂-Ph | NH—(R)—CHCH₃—C(CH₃)₃ | CO—(O—CH₂—CH₂)₂—OCH₃ | H |
| I-3-256 | Cl | 2,4-Cl₂-Ph | NH—(R)—CHCH₃—C(CH₃)₃ | CO₂—O—CH₂—CO₂CH₃ | H |
| I-3-257 | Cl | 2-Cl, 4-F-Ph | NH—CH(CH₃)₂ | CO—(O—CH₂—CH₂)₂—OCH₃ | H |
| I-3-258 | Cl | 2-Cl-Ph | NH—CH(CH₃)₂ | CO—O—CH₂—CO₂CH₃ | H |
| I-3-259 | Cl | 2-Cl, 4-F-Ph | NH—(R)—CHCH₃—C(CH₃)₃ | CO—O—CH₂—CHOH—CH₂—OH | H |
| I-3-260 | Cl | 2,4,5-F₃-Ph | NH—(R)—CHCH₃—C(CH₃)₃ | CO—O—CH₂—CO—NCH₃—OCH₃ | H |
| I-3-261 | Cl | 2,4,5-F₃-Ph | NH—(S)—CH(CF₃)(CH₃) | CO₂CH₃ | H |
| I-3-262 | Cl | 2-F, 4-CH₃-Ph | NH—(R)—CHCH₃—CH(CH₃)₂ | CO—O—(CH₂)₂—OCH₃ | H |
| I-3-263 | Cl | 2,5-F₂-Ph | NH—(R)—CHCH₃—CH(CH₃)₂ | CO₂CH₃ | H |
| I-3-264 | Cl | 2-Br, 4-F-Ph | NH—(S)—CHCH₃—CH(CH₃)₂ | 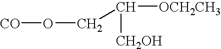 | H |
| I-3-265 | Cl | 2-Br, 4-F-Ph | NH—(S)—CHCH₃—CH(CH₃)₂ | CO—O—(CH₂)₃—N(C₂H₅)₂ | H |
| I-3-266 | Cl | 2-Br, 4-F-Ph | NH—(S)—CHCH₃—CH(CH₃)₂ | CO—O—CH₂—CO₂CH₃ | H |
| I-3-267 | Cl | 2-Br, 4-F-Ph | NH—(S)—CHCH₃—CH(CH₃)₂ | 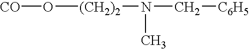 | H |
| I-3-268 | Cl | 2-Br, 4-F-Ph | NH—(S)—CHCH₃—C(CH₃)₃ | CO—O—CH(C₂H₅)—CH₂—OCH₃ | H |
| I-3-269 | Cl | 2,5-F₂-Ph | NH—(R)—CHCH₃—CH(CH₃)₂ | CO—O—CH(CF₃)—CO₂C₂H₅ | H |
| I-3-270 | Cl | 2,5-F₂-Ph | NH—(R)—CHCH₃—CH(CH₃)₂ | CO—O—CHCH₃—CH₂—N(CH₃)₂ | H |
| I-3-271 | Cl | 2,5-F₂-Ph | NH—(R)—CHCH₃—C(CH₃)₃ | CO—O—CHCH₃—CH₂—N(C₂H₅)₂ | H |
| I-3-272 | Cl | 2-Cl, 5-F-Ph | NH—(R)—CHCH₃—C(CH₃)₃ | CO—O—CH(CH₂CH₃)—CH₂—OCH₃ | H |
| I-3-273 | Cl | 2-Cl, 5-F-Ph | NH—(R)—CHCH₃—C(CH₃)₃ | 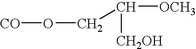 | H |
| I-3-274 | Cl | 2-Cl, 5-F-Ph | NH—(R)—CHCH₃—C(CH₃)₃ | 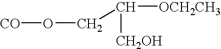 | H |
| I-3-275 | Cl | 2-Cl, 5-F-Ph | NH—(R)—CHCH₃—C(CH₃)₃ | CO—O—CH(CH₂OCH₃)₂ | H |
| I-3-276 | Cl | 2-Cl, 5-F-Ph | NH—(R)—CHCH₃—C(CH₃)₃ | CO—O—CH(CH₂OCH₂CH₃)₂ | H |
| I-3-277 | Cl | 2-Cl, 5-F-Ph | NH—(R)—CHCH₃—C(CH₃)₃ | CO—O—CHCH₃—CH₂OCH₂CH₃ | H |
| I-3-278 | Cl | 2-Cl, 5-F-Ph | NH—(R)—CHCH₃—C(CH₃)₃ | 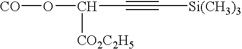 | H |
| I-3-279 | Cl | 2,5-F₂-Ph | NH—(R)—CHCH₃—CH(CH₃)₂ | CO—O—(CH₂)₃—N(CH₃)₂ | H |
| I-3-280 | Cl | 2,5-F₂-Ph | NH—(R)—CHCH₃—CH(CH₃)₂ | 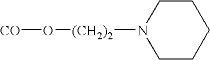 | H |
| I-3-281 | Cl | 2-Br, 4-F-Ph | NH—(S)—CHCH₃—C(CH₃)₃ | CO—O—CH₂-(S)-CHCH₃—OCH₃ | H |
| I-3-282 | Cl | 2-Br, 4-F-Ph | NH—(S)—CHCH₃—C(CH₃)₃ | 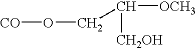 | H |
| I-3-283 | Cl | 2-Br, 4-F-Ph | NH—(S)—CHCH₃—C(CH₃)₃ | 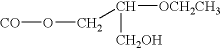 | H |
| I-3-284 | Cl | 2-Br, 4-F-Ph | NH—(S)—CHCH₃—C(CH₃)₃ | 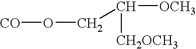 | H |
| I-3-285 | Cl | 2-Br, 4-F-Ph | NH—(S)—CHCH₃—C(CH₃)₃ | 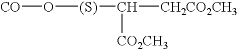 | H |
| I-3-286 | Cl | 2-Br, 4-F-Ph | NH—(S)—CHCH₃—C(CH₃)₃ | 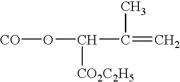 | H |
| I-3-287 | Cl | 2-Br, 4-F-Ph | NH—(S)—CHCH₃—C(CH₃)₃ | 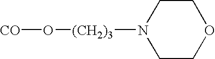 | H |
| I-3-288 | Cl | 2-Br, 4-F-Ph | NH—(S)—CHCH₃—C(CH₃)₃ | CO—O—(CH₂)₃—N(CH₃)₂ | H |
| I-3-289 | Cl | 2-Br, 4-F-PH | NH—(S)—CHCH₃—CH(CH₃)₂ | 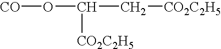 | H |
| I-3-290 | Cl | 2-Br, 4-F-Ph | NH—(S)—CHCH₃—C(CH₃)₃ | CO—O—CHCH₃—CH₂—N(C₂H₅)₂ | H |
| I-3-291 | Cl | 2-Br, 4-F-Ph | NH—(S)—CHCH₃C(CH₃)₃ | CO—O—CHCH₃—CH₂—N(CH₃)₂ | H |
| I-3-292 | Cl | 2-Br, 4-F-Ph | NH—(S)—CHCH₃—C(CH₃)₃ | 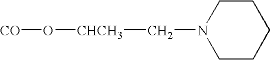 | H |
| I-3-293 | Cl | 2-Br, 4-F-Ph | NH—(S)—CHCH₃—CH(CH₃)₂ | 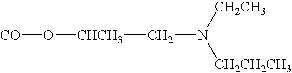 | H |
| I-3-294 | Cl | 2-Cl, 5-F-Ph | NH—(S)—CHCH₃—CH₂CH₃ | CO₂CH₃ | H |
| I-3-295 | Cl | 2,5-F₂-Ph | NH—(R)—CHCH₃—CH₂CH₃ | CO₂CH₃ | H |
| I-3-296 | Cl | 2,5-F₂Ph | NH—(S)—CHCH₃—CH₂CH₃ | CO₂CH₃ | H |
| I-3-297 | Cl | 2-Br, 4-F-Ph | NH—(R)—CHCH₃—CH₂CH₃ | 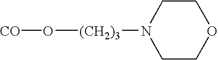 | H |
| I-3-298 | Cl | 2-Cl, 5-F-Ph | NH—(R)—CHCH₃—C(CH₃)₃ | CO—O—(CH₂)₃—N(C₂H₅)₂ | H |
| I-3-299 | Cl | 2-Cl, 5-F-Ph | NH—(R)—CHCH₃—C(CH₃)₃ | 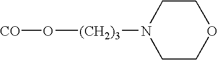 | H |
| I-3-300 | Cl | 2-Cl, 5-F-Ph | NH—(R)—CHCH₃—C(CH₃)₃ | CO—O—CHCH₃—CH₂—N(C₂H₅)₂ | H |
| I-3-301 | Cl | 2-Cl, 5-F-Ph | NH—(R)—CHCH₃—C(CH₃)₃ | CO—O—(CH₂)₂—N(CH₃)₂ | H |
| I-3-302 | Cl | 4-Cl, 2-F-Ph | NH—(R)—CHCH₃—C(CH₃)₃ | 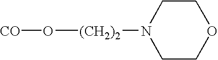 | H |
| I-3-303 | Cl | 4-Cl, 2-F-Ph | NH—(R)—CHCH₃—C(CH₃)₃ | 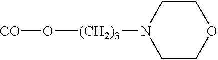 | H |
| I-3-304 | Cl | 4-Cl, 2-F-Ph | NH—(R)—CHCH₃—C(CH₃)₃ | CO—O—(CH₂)₂—N(CH₃)₂ | H |
| I-3-305 | Cl | 2-Cl, 5-CH₃-Ph | NH—(R)—CHCH₃—C(CH₃)₃ | 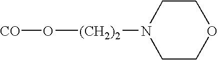 | H |
| I-3-306 | Cl | 2-Cl, 4-F-Ph | NH—(R)—CHCH₃—C(CH₃)₃ | CO₂CH₃ | CH₃ |
| I-3-307 | Cl | 2,4-F₂-Ph | NH—(R)—CHCH₃—CH(CH₃)₂ | CO—O—CH₂—CO—NCH₃—OCH₃ | H |
| I-3-308 | Cl | 2,4-F₂-Ph | NH—(R)—CHCH₃—C(CH₃)₃ | CO—(O—CH₂—CH₂)₃—OCH₃ | H |
| I-3-309 | Cl | 2,4-F₂-Ph | NH—(R)—CHCH₃—C(CH₃)₃ | CO—(O—CH₂—CH₂)₂—OCH₃ | H |
| I-3-310 | Cl | 2-Br-Ph | NH—(S)—CH(CF₃)(CH₃) | CO₂CH₃ | H |
| I-3-311 | Cl | 2-F-Ph | NH—(R)—CHCH₃—CH(CH₃)₂ | CO₂CH₃ | H |
| I-3-312 | Cl | 2-Cl, 4-F-Ph | NH—(R)—CHCH₃—C(CH₃)₃ | CO₂CH₃ | H |
| I-3-313 | Cl | 2-Cl-Ph | NH—(S)—CH(CF₃)(CH₃) | CO₂CH₃ | H |
| I-3-314 | Cl | 2-Cl, 6-F-Ph | NH—(S)—CH(CF₃)(CH₃) | CO₂CH₃ | H |
| I-3-315 | Cl | 2-Cl, 6-F-Ph | NH—(R)—CHCH₃—C(CH₃)₃ | CO₂CH₂CH₃ | H |
| I-3-316 | Cl | 2-Cl, 6-F-Ph | NH—(R)—CHCH₃—C(CH₃)₃ | CO₂CH₂CH═CH₂ | H |
| I-3-317 | Cl | 2-Cl, 6-F-Ph | NH—(R)—CHCH₃—C(CH₃)₃ | CO₂(CH₂)₂OCH₃ | H |
| I-3-318 | Cl | 2-Cl, 4-F-Ph | NH—(R)—CHCH₃—C(CH₃)₃ | CO₂(CH₂)₂OCH₃ | H |
| I-3-319 | Cl | 2,4-Cl₂-Ph | NH—(S)—CH(CF₃)(CH₃) | CO₂CH₃ | H |
| I-3-320 | Cl | 2-Cl, 4-F-Ph | NH—(R)—CHCH₃—C(CH₃)₃ | CO—O—CH₂—CO—NCH₃—OCH₃ | H |
| I-3-321 | Cl | 2-Cl-Ph | NH—(S)—CH(CF₃)(CH₃) | CO₂(CH₂)₂OCH₃ | H |
| I-3-322 | Cl | 2-Cl, 4-F-Ph | NH—(S)—CH(CF₃)(CH₃) | CO₂CH₂CH₃ | H |
| I-3-323 | Cl | 2,6-F₂-Ph | NH—CH(CH₃)₂ | CO—NH—SO₂—CH₃ | H |
| I-3-324 | Cl | 2-Cl, 4-F-Ph | NH—(S)—CH(CF₃)(CH₃) | CO₂(CH₂)₂OCH₃ | H |
| I-3-325 | Cl | 2-Cl, 4-F-Ph | NH—(S)—CH(CF₃)(CH₃) | CO₂CH₂CH═CH₂ | H |
| I-3-326 | Cl | 2-Cl-Ph | NH—(R)—CHCH₃—CH(CH₃)₂ | CO₂CH₂CH₃ | H |
| I-3-327 | Cl | 2-Cl-Ph | NH—(R)—CHCH₃—CH(CH₃)₂ | CO₂CH₂CH═CH₂ | H |
| I-3-328 | Cl | 2,4-Cl₂-Ph | NH—(R)—CHCH₃—C(CH₃)₃ | CO₂CH₃ | H |
| I-3-329 | Cl | 2-Cl, 4-F-Ph | NH—(R)—CHCH₃—C(CH₃)₃ | CO—O—CHCH₃—CO₂CH₃ | H |
| I-3-330 | Cl | 2-Cl, 4-F-Ph | NH—(R)—CHCH₃—C(CH₃)₃ | CO—O—CH₂—CO₂CH₃ | H |
| I-3-331 | Cl | 4-Cl, 2,6-F₂-Ph | NH—(R)—CHCH₃—C(CH₃)₃ | CO₂CH₃ | H |
| I-3-332 | Cl | 2-Cl, 6-F-Ph | NH—CH(CH₃)₂ | CO₂CH₃ | H |
| I-3-333 | Cl | 2-Cl-Ph | NH—CH(CH₃)₂ | CO₂CH₃ | H |
| I-3-334 | Cl | 4-Cl, 2,6-F₂-Ph | NH—(R)—CHCH₃—CH(CH₃)₂ | CO₂CH₃ | H |
| I-3-335 | Cl | 4-Cl, 2,6-F₂-Ph | NH—CH(CH₃)₂ | CO₂CH₃ | H |
| I-3-336 | Cl | 2,4,6-F₃-Ph | NH—(R)—CHCH₃—CH(CH₃)₂ | CO—O—CH₂—CO—NCH₃—OCH₃ | H |
| I-3-337 | Cl | 2,4-Cl₂-Ph | NH—(R)—CHCH₃—CH(CH₃)₂ | CO—O—CHCH₃—CO₂CH₃ | H |
| I-3-338 | Cl | 2,4-Cl₂-Ph | NH—(R)—CHCH₃—CH(CH₃)₂ | CO—O-(S)-CHCH₃—CO—NHCH₃ | H |
| I-3-339 | Cl | 2,4,6-F₃-Ph | NH—(R)—CHCH₃—C(CH₃)₃ | CO—O—CH₂—CO₂CH₃ | H |
| I-3-340 | Cl | 2,4,6-F₃-Ph | NH—CH(CH₃)₂ | CO—O—CHCH₃—CO₂CH₃ | H |
| I-3-341 | Cl | 2-Cl-Ph | NH—(R)—CHCH₃—C(CH₃)₃ | CO—O—CHCH₃—CO₂CH₃ | H |
| I-3-342 | Cl | 2-Cl, 6-F-Ph | NH—(R)—CHCH₃—CH(CH₃)₂ | CO—O—CHCH₃—CO₂CH₃ | H |
| I-3-343 | Cl | 2-Cl, 6-F-Ph | NH—(R)—CHCH₃—C(CH₃)₃ | CO—(O—CH₂—CH₂)₂—OCH₃ | H |
| I-3-344 | Cl | 2-Cl-Ph | NH—(R)—CHCH₃—C(CH₃)₃ | CO—O—CH₂—CO₂CH₃ | H |
| I-3-345 | Cl | 2-Cl, 6-F-Ph | NH—(R)—CHCH₃—CH(CH₃)₂ | CO—O—CH₂—CO₂CH₃ | H |
| I-3-346 | Cl | 2-Cl, 6-F-Ph | NH—(R)—CHCH₃—C(CH₃)₃ | CO—O—CH₂—CO₂CH₃ | H |
| I-3-347 | Cl | 2-Cl, 4-F-Ph | NH—(R)—CHCH₃—C(CH₃)₃ | CO₂CH₃ | Cl |
| I-3-348 | Cl | 2,5-F₂-Ph | NH—(R)—CHCH₃—C(CH₃)₃ | CO₂CH₃ | H |
| I-3-349 | Cl | 2,5-F₂-Ph | NH—(R)—CHCH₃—CH(CH₃)₂ | CO—O—CH₂—CH═CH₂ | H |
| I-3-359 | Cl | 2,5-F₂-Ph | NH—(R)—CHCH₃—CH(CH₃)₂ | CO₂CH₂CH₃ | H |
| I-3-360 | Cl | 2,5-F₂-Ph | NH—CH(CH₃)₂ | CO₂CH₃ | H |
| I-3-361 | Cl | 2-Cl, 5-F-Ph | NH—(S)—CH—(CF₃)—CH₃ | CO₂CH₃ | H |
| I-3-362 | Cl | 2-Cl, 5-F-Ph | NH—(R)—CHCH₃—CH(CH₃)₂ | CO—O—(CH₂)₃—N(C₂H₅)₂ | H |
| I-3-363 | Cl | 2-Cl, 5-F-Ph | NH—(R)—CHCH₃—CH(CH₃)₂ | 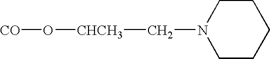 | H |
| I-3-364 | Cl | 4-Cl, 2-F-Ph | NH—(R)—CHCH₃—C(CH₃)₃ | CO—O—CHCH₃—CH₂—N(C₂H₅)₂ | H |
| I-3-365 | Cl | 4-Cl, 2-F-Ph | NH—(R)—CHCH₃—C(CH₃)₃ | CO—O—(CH₂)₃—N(C₂H₅)₂ | H |
| I-3-366 | Cl | 2,5-F₂-Ph | NH—(R)—CHCH₃—CH₂CH₃ | CO—O—(CH₂)₃—N(C₂H₅)₂ | H |
| I-3-367 | Cl | 4-Cl, 2-F-Ph | NH—(R)—CHCH₃—CH₂CH₃ | CO—O—(CH₂)₃—N(C₂H₅)₂ | H |
| I-3-368 | Cl | 2-Br, 4-F-Ph | NH—(R)—CHCH₃—CH₂CH₃ | CO—O—(CH₂)₃—N(C₂H₅)₂ | H |
| I-3-369 | Cl | 2-Cl, 5-F-Ph | NH—(R)—CHCH₃—CH₂CH₃ | CO—O—(CH₂)₃—N(C₂H₅)₂ | H |
| I-3-370 | Cl | 2,3,6-F₃-Ph | NH—(R)—CHCH₃—C(CH₃)₃ | CO₂CH₃ | Cl |
| I-3-371 | Cl | 2,4-F₂-Ph | NH—(R)—CHCH₃—C(CH₃)₃ | CO₂CH₂CH₃ | H |
| I-3-372 | Cl | 2,4-F₂-Ph | NH—(R)—CHCH₃—C(CH₃)₃ | CO₂CH₂C≡CH | H |
| I-3-373 | Cl | 2-Cl, 5-F-Ph | NH—CH(CH₃)₂ | CO₂CH₃ | H |
| I-3-374 | Cl | 2,5-F₂-Ph | NH—(R)—CHCH₃—CH(CH₃)₂ | CO—NH—CH₂—CO₂CH₃ | H |
| I-3-375 | Cl | 2-Cl, 5-F-Ph | NH—(R)—CHCH₃—C(CH₃)₃ | CO—NH—CH₂—CO₂CH₂CH₂CH₃ | H |
| I-3-376 | Cl | 2-Cl, 5-F-Ph | NH—(R)—CHCH₃—C(CH₃)₃ | CO—NH—CHCH₃—CO₂CH₂CH₃ | H |
| I-3-377 | Cl | 2,5-Fl₂, 4-CH₃-Ph | NH—(R)—CHCH₃—CH(CH₃)₂ | CO—NH—CH(CH₃)₂ | H |
| I-3-378 | Cl | 2,4,5-F₃-Ph | NH—(R)—CHCH₃—CH(CH₃)₂ | CO—NH—SO₂—CH₃ | H |
| I-3-379 | Cl | 2,4,5-F₃-Ph | NH—(S)—CH—(CF₃)—CH₃ | CO—NH—SO₂—CH₃ | H |
| I-3-380 | Cl | 2-Cl-Ph | NH—CH₂—CH(CH₃)₂ | CO—NH—SO₂—CH₃ | H |
| I-3-381 | Cl | 2-Cl, 4-F-Ph | NH—(R)—CHCH₃—CH(CH₃)₂ | CO—NH—SO₂—CH₃ | Cl |
| I-3-382 | Cl | 2-Cl, 4-F-Ph | NH—(R)—CHCH₃—C(CH₃)₃ | CO—NH—SO₂—CH₃ | Cl |
| I-3-383 | Cl | 2-Cl, 4-F-Ph | NH—(R)—CHCH₃—C(CH₃)₃ | CO—NH—SO₂—CH(CH₃)₂ | CF₃ |
| I-3-384 | Cl | 2-Br-Ph | NH—CH(CH₃)₂ | CO—NH—SO₂—CH₃ | H |
| I-3-385 | Cl | 2-F-Ph | NH—CH(CH₃)₂ | CO—NH—SO₂—CH₃ | H |
| I-3-386 | Cl | Ph | NH—CH(CH₃)₂ | CO—NH—SO₂—CH₃ | H |
| I-3-387 | Cl | 4-Cl-Ph | NH—CH(CH₃)₂ | CO—NH—SO₂—CH₃ | H |
| I-3-388 | Cl | 4-Cl-Ph | NH—CH(CH₃)₂ | CO—NH—SO₂—CH₂CH₃ | H |
| I-3-389 | Cl | 2,3-Cl-Ph | NH—CH(CH₃)₂ | CO—NH—SO₂—CH₃ | H |
| I-3-390 | Cl | 2,4,6-F₃-Ph | NH—CH(CH₃)₂ | CO—NH—SO₂—CH₂CH₃ | H |
| I-3-391 | Cl | 2,4,6-F₃-Ph | NH—CH(CH₃)₂ | CO—NH—SO₂—CH(CH₃)₂ | H |
| I-3-392 | Cl | 2,4,6-F₃-Ph | NH—CH(CH₃)₂ | CO—NH—SO₂—CH₂CF₃ | H |
| I-3-393 | Cl | 2,4,6-F₃-Ph | NH—CH(CH₃)₂ | CO—NH—SO₂—CH₂Ph | H |
| I-3-394 | Cl | 2,4,6-F₃-Ph | NH—CH(CH₃)₂ | CO—NH—SO₂—C(CH₃)₃ | H |
| I-3-395 | Cl | 2,4,6-F₃-Ph | NH—CH(CH₃)₂ | 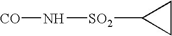 | H |
| I-3-396 | Cl | 2,4,6-F₃-Ph | 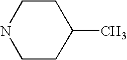 | CO—NH—SO₂—CH₃ | H |
| I-3-397 | Cl | 2,4-Cl₂, 5-F-Ph | NH—(R)—CHCH₃—CH(CH₃)₂ | CO—NCH₃—OCH₃ | H |
| I-3-398 | Cl | 2,4-Cl₂, 5-F-Ph | NH—(R)—CHCH₃—C(CH₃)₂ | CO—NCH₃—OCH₃ | H |
| I-3-399 | Cl | 2,4,6-F₃-Ph | NH—CH₂—CH(CH₃)₂ | CO—NCH₃—OCH₃ | H |
| I-3-400 | Cl | 4-Cl, 2-F-Ph | NH—(S)—CHCH₃—CH(CH₃)₂ | CO—NH—OCH₂CH₂Cl | H |
| I-3-401 | Cl | 2-Cl, 5-F-Ph | NH—(R)—CHCH₃—C(CH₃)₃ | CO—NCH₃—OCH₃ | H |
| I-3-402 | Cl | 2-Br, 4-F-Ph | NH—(R)—CHCH₃—C(CH₃)₃ | CO—NCH₃—OCH₃ | H |
| I-3-403 | Cl | 2,5-F₂-Ph | NH—(R)—CHCH₃—CH₂CH₃ | CO—NCH₃—OCH₃ | H |
| I-3-404 | Cl | 2,4,6-F₃-Ph | NH—(R)—CHCH₃—C(CH₃)₃ | CO—NCH₃—OCH₃ | H |
| I-3-405 | Cl | 2,4-Cl₂-Ph | NH—CH(CH₃)₂ | CO—NCH₃—OCH₃ | H |
| I-3-406 | Cl | 4-Cl, 2,6-F₂-Ph | NH—CH(CH₃)₂ | CO—NCH₃—OCH₃ | H |
| I-3-407 | Cl | 2,5-F₂-Ph | NH—(R)—CHCH₃—C(CH₃)₃ | CO—NCH₃—OCH₃ | H |
| I-3-408 | Cl | 2,4-Cl₂-Ph | NH—(S)—CH(CF₃)(CH₃) | CO—NH—SO₂—CH₃ | H |
| I-3-409 | Cl | 4-Cl, 2,6-F₂-Ph | NH—(S)—CH(CF₃)(CH₃) | CO—NH—SO₂—CH₃ | H |
| I-3-410 | Cl | 2,4,6-F₃-Ph | 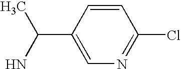 | CO—NH—SO₂—CH₃ | H |

*Ph = phenyl

### EXAMPLE 1

Phaedon test (spray treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Discs of chinese cabbage leaves (Brassica pekinensis) are sprayed with an active compound preparation of the desired concentration and, after drying, populated with larvae of the mustard beetle (Phaedon cochleariae).

After the desired period of time, the effect in % is determined. 100% means that all beetle larvae have been killed, 0% means that none of the beetle larvae have been killed.

In this test, for example, the following compounds of the preparation examples show, at an application rate of 500 g/ha, an efficacy of ≧80%: I-3-1, I-3-3, I-3-4, I-3-5, I-3-6, I-3-7, I-3-9, I-3-10, I-3-11, I-3-12, I-3-13, I-3-14, I-3-15, I-3-16, I-3-17, I-3-19, I-3-21, I-3-22, I-3-25, I-3-26, I-3-27, I-3-28, I-3-29, I-3-30, I-3-31, I-3-32, I-3-35, I-3-37, I-3-38, I-3-39, I-3-42, I-3-47, I-3-48, I-3-49, I-3-50, I-3-52, I-3-53, I-3-54, I-3-55, I-3-56, I-3-57, I-3-58, I-3-59, I-3-60, I-3-61, I-3-62, I-3-63, I-3-64, I-3-65, I-3-66, I-3-67, I-3-68, I-3-70, I-3-71, I-3-72, I-3-73, I-3-74, I-3-75, I-3-76, I-3-77, I-3-78, I-3-79, I-3-80, I-3-81, I-3-82, I-3-83, I-3-85, I-3-86, I-3-87, I-3-88, I-3-89, I-3-90, I-3-91, I-3-92, I-3-93, I-3-94, I-3-95, I-3-96, I-3-98, I-3-99, I-3-101, I-3-102, I-3-104, I-3-109, I-3-110, I-3-111, I-3-112, I-3-113, I-3-114, I-3-115, I-3-116, I-3-117, I-3-118, I-3-119, I-3-120, I-3-121, I-3-122, I-3-123, I-3-124, I-3-125, I-3-127, I-3-128, I-3-129, I-3-131, I-3-132, I-3-133, I-3-135, I-3-136, I-3-137, I-3-138, I-3-139, I-3-140, I-3-141, I-3-142, I-3-144, I-3-145, I-3-146, I3-147, I-3-148, I-3-149, I-3-150, I-3-152, I-3-154, I-3-155, I-3-156, I-3-157, I-3-158, I-3-159, I-3-160, I-3-161, I-3-163, I-3-164, I-3-165, I-3-166, I-3-167, I-3-168, I-3-169, I-3-170, I-3-172, I-3-173, I-3-174, I-3-175, I-3-176, I-3-177, I-3-179, I-3-180, I-3-181, I-3-182, I-3-183, I-3-184, I-3-185, I-3-186, I-3-187, I-3-188, I-3-189, I-3-190, I-3-191, I-3-192, I-3-193, I-3-194, I-3-195, I-3-196, I-3-197, I-3-198, I-3-199, I-3-200, I-3-201, I-3-202, I-3-203, I-3-204, I-3-205, I-3-206, I-3-207, I-3-208, I-3-209, I-3-210, I-3-211, I-3-212, I-3-213, I-3-214, I-3-215, I-3-216, I-3-217, I-3-218, I-3-219, I-3-220, I-3-221, I-3-222, I-3-223, I-3-224, I-3-225, I-3-226, I-3-227, I-3-228, I-3-229, I-3-230, I-3-231, I-3-232, I-3-233, I-3-234, I-3-235, I-3-236, I-3-237, I-3-238, I-3-239, I-3-240, I-3-241, I-3-242, I-3-243, I-3-244, I-3-245, I-3-246, I-3-247, I-3-248, I-3-249, I-3-251, I-3-252, I-3-253, I-3-254, I-3-255, I-3-256, I-3-257, I-3-258, I-3-259, I-3-260, I-263, I-3-264, I-3-265, I-3-266, I-3-267, I-3-268, I-3-269, I-3-270, I-3-272, I-3-273, I-3-274, I-3-275, I-3-276, I-3-277, I-3-278, I-3-279, I-3-280, I-3-281, I-3-282, I-3-283, I-3-284, I-3-285, I-3-286, I-3-287, I-3-288, I-3-289, I-3-290, I-3-291, I-3-292, I-3-293, I-3-294, I-3-294, I-3-295, I-3-296, I-3-297, I-3-298, I-3-299, I-3-300, I-3-301, I-3-302, I-3-303, I-3-304, I-3-305, I-3-307, I-3-308, I-3-309, I-3-310, I-3-313, I-3-314, I-3-315, I-3-316, I-3-317, I-3-318, I-3-319, I-3-320, I-3-321, I-3-322, I-3-323, I-3-324, I-3-325, I-3-326, I-3-327, I-3-328, I-3-329, I-3-330, I-3-331, I-3-332, I-3-333, I-3-335, I-3-336, I-3-337, I-3-338, I-3-339, I-3-340, I-3-342, I-3-343, I-3-344, I-3-345, I-3-346, I-3-348, I-3-349, I-3-359, I-3-360, I-3-361, I-3-362, I-3-363, I-3-364, I-3-365, I-3-366, I-3-367, I-3-368, I-3-369, I-3-370, I-3-371, I-3-372, I-3-373, I-3-374, I-3-375, I-3-376, I-3-377, I-3-378, I-3-379, I-3-380, I-3-381, I-3-382, I-3-383, I-3-384, I-3-385, I-3-386, I-3-387, I-3-388, I-3-389, I-3-390, I-3-391, I-3-392, I-3-393, I-3-394, I-3-395, I-3-396, I-3-397, I-3-398, I-3-399, I-3-401, I-402, I-3-403, I-3-404, I-3-406, I-3-407, I-3-408, I-3-409-I-3-410.

### Example 2

Myzus test (spray treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Discs of chinese cabbage leaves (Brassica pekinensis) which are infested by all stages of the green peach aphid (Myzus persicae) are sprayed with an active compound preparation of the desired concentration.

After the desired period of time, the effect in % is determined. 100% means that all aphids have been killed; 0% means that none of the aphids have been killed.

In this test, for example, the following compounds of the preparation examples show, at an application rate of 500 g/ha, an efficacy of ≧80%: I-3-5, I-3-6, I-3-7, I-3-8, I-3-9, I-3-11, I-3-13, I-3-16, I-3-31, I-3-32, I-3-35, I-3-43, I-3-72, I-3-84, I-3-87, I-3-124, I-3-184, I-3-201, I-3-260, I-3-268, I-3-323, I-3-349, I-3-378, I-3-379, I-3-384, I-3-385, I-3-386, I-3-389, I-3-390, I-3-395, I-3-408, I-3-409, I-3-410.

### Example 3

Spodoptera frugiperda test (spray treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Discs of maize leaves (Zea mays) are sprayed with an active compound preparation of the desired concentration and, after drying, populated with caterpillars of the armyworm (Spodoptera frugiperda).

After the desired period of time, the effect in % is determined. 100% means that all caterpillars have been killed; 0% means that none of the caterpillars have been killed.

In this test, for example, the following compounds of the preparation examples show, at an application rate of 500 g/ha, an efficacy of >80%: I-3-1, I-3-5, I-3-6, I-3-8, I-3-9,1-3-10, I-3-11, I-3-12, I-3-13, I-3-14, I-3-15, I-3-16, I-3-17, I-3-21, I-3-23, I-3-24, I-3-25, I-3-27, I-3-28, I-3-30, I-3-31, I-3-32, I-3-33, I-3-35, I-3-36, I-3-37, I-3-38, I-3-45, I-3-46, I-3-47, I-3-48, I-3-49, I-3-50, I-3-51, I-3-58, I-3-59, I-3-60, I-3-61, I-3-62, I-3-64, I-3-66, I-3-67, I-3-68, I-3-71, I-3-72, I-3-76, I-3-79, I-3-81, I-3-84, I-3-86, I-3-87, I-3-88, I-3-89, I-3-91, I-3-92, I-3-94, I-3-96, I-3-101, I-3-102, I-3-109, I-3-112, I-3-113, I-3-114, I-3-115, I-3-116, I-3-117, I-3-118, I-3-119, I-3-121, I-3-122, I-3-123, I-3-124, I-3-125, I-3-127, I-3-128, I-3-129, I-3-140, I-3-141, I-3-142, I-3-143, I-3-144, I-3-145, I-3-146, I-3-147, I-3-149, I-3-150, I-3-152, I-3-154, I-3-155, I-3-156, I-3-157, I-3-158, I-3-159, I-3-160, I-3-161, I-3-164, I-3-165, I-3-174, I-3-176, I-3-181, I-3-182, I-3-183, I-3-186, I-3-187, I-3-188, I-3-191, I-3-192, I-3-193, I-3-198, I-3-202, I-3-203, I-3-205, I-3-206, I-3-207, I-3-214, I-3-215, I-3-216, I-3-217, I-3-218, I-3-220, I-3-221, I-3-224, I-3-225, I-3-236, I-3-242, I-3-245, I-3-246, I-3-247, I-3-248, I-3-249, I-3-250, I-3-251, I-3-254, I-3-255, I-3-256, I-3-257, I-3-259, I-3-260, I-3-262, I-3-263, I-3-280, I-3-281, I-3-287, I-3-298, I-3-299, I-3-300, I-3-302, I-3-304, I-3-306, I-3-308, I-3-309, I-3-312, I-3-317, I-3-318, I-3-319, I-3-320, I-3-322, I-3-325, I-3-327, I-3-328, I-3-329, I-3-330, I-3-331, I-3-334, I-3-336, I-3-337, I-3-341, I-3-342, I-3-346, I-3-347, I-3-348, I-3-349, I-3-361, I-3-365, I-3-370, I-3-371, I-3-372, I-3-378, I-3-381, I-3-382, I-3-397, I-3-400, I-3-401, I-3-402, I-3-404, I-3-408, I-3-409.

### Example 4

Tetranychus test, OP-resistant (spray treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Discs of bean leaves (Phaseolus vulgaris) infested by all stages of the greenhouse red spider mite (Tetranychus urticae) are sprayed with an active compound preparation of the desired concentration.

After the desired period of time, the effect in % is determined. 100% means that all spider mites have been killed; 0% means that none of the spider mites have been killed.

In this test, for example, the following compounds of the preparation examples show, at an application rate of 100 g/ha, an efficacy of ≧80%: I-3-58, I-3-76, I-3-315, I-3-316, I-3-318, I-3-329, I-3-330, I-3-346, I-3-348.

In this test, for example, the following compounds of the preparation examples show, at an application rate of 500 g/ha, an efficacy of ≧80%: I-3-6, I-3-11, I-3-14, I-3-17, I-3-27, I-3-38, I-3-68, I-3-79, I-3-81, I-3-92, I-3-125, I-3-246, I-3-248, I-3-251, I-3-260, I-3-262, I-3-283, I-3-301, I-3-302, I-3-309, I-3-388, I-3-389, I-3-320, I-3-334, I-3-337, I-3-340, I-3-348, I-3-359, I-3-362, I-3-365, I-3-382, I-3-401, I-3-407, I-3-410.

### Example 5

Lucilia cuprina test

| | |
|---|---|
| Solvent: | dimethyl sulphoxide |

To produce a suitable preparation of active compound, 1 part by weight of active compound is mixed with the stated amount of water, and the concentrate is diluted with water to the desired concentration.

Vessels containing horse meat which had been treated with the preparation of active compound of the desired concentration are populated with Lucilia cuprina larvae.

After the desired period of time, the kill in % is determined. 100% means that all larvae have been killed; 0% means that none of the larvae have been killed.

In this test, for example, the following compounds of the preparation examples show, at a test concentration of 100 ppm, an efficacy of ≧80%: I-3-1, I-3-6, I-3-7, I-3-8, I-3-9, I-3-10, I-3-11, I-3-12, I-3-13, I-3-14, I-3-15, I-3-16, I-3-17, I-3-18, I-3-19, I-3-21, I-3-22, I-3-23, I-3-24, I-3-25, I-3-26, I-3-27, I-3-28, I-3-30, I-3-31, I-3-32, I-3-33, I-3-35, I-3-36, I-3-37, I-3-38, I-3-63, I-3-67, I-3-76, I-3-127, I-3-129, I-3-149, I-3-188, I-3-205, I-3-214, I-3-225, I-3-246, I-3-248, I-3-251, I-3-260, I-3-323, I-3-339, I-3-345, I-3-346, I-3-378, I-3-379, I-3-381, I-3-382, I-3-384, I-3-385, I-3-386, I-3-387, I-3-389, I-3-390, I-3-392, I-3-408, I-3-409.

### Example 6

Boophilus microplus test

| | |
|---|---|
| Solvent: | dimethyl sulphoxide |

To produce a suitable preparation of active compound, 1 part by weight of active compound is mixed with the stated amount of solvent, and the concentrate is diluted with solvent to the desired concentration.

The solution of active compound is injected into the abdomen (Boophilus microplus), the animals are transferred into dishes and stored in a climatized room.

After the desired period of time, the effect in % is determined. 100% means that none of the ticks has laid any fertile eggs.

In this test, for example, the following compounds show, at an application rate of 20 μg/animal, an effect of ≧80%: I-3-1, I-3-4, I-3-11, I-3-12, I-3-17, I-3-18, I-3-27, I-3-28, I-3-31, I-3-33, I-3-35, I-3-36, I-3-37, I-3-38, I-3-46, I-3-58, I-3-67, I-3-74, I-3-76, I-3-88, I-3-92, I-3-114, I-3-119, I-3-122, I-3-123, I-3-127, I-3-128, I-3-129, I-3-147, I-3-149, I-3-159, I-3-172, I-3-176, I-3-185, I-3-187, I-3-188, I-3-202, I-3-205, I-3-214, I-3-225, I-3-227, I-3-228, I-3-229, I-3-231, I-3-246, I-3-248, I-3-251, I-3-259, I-3-260, I-3-302, I-3-312, I-3-326, I-3-328, I-3-337, I-3-339, I-3-346, I-3-378, I-3-381, I-3-382, I-3-389.

### Example 7

Musca domestica test

| | |
|---|---|
| Solvent: | dimethyl sulphoxide |

To produce a suitable preparation of active compound, 1 part by weight of active compound is mixed with the stated amount of water, and the concentrate is diluted with water to the desired concentration.

Vessels containing a sponge treated with the active compound preparation of the desired concentration are populated with (Musca domestica) adults.

After the desired period of time, the kill in % is determined. 100% means that all flies have been killed; 0% means that none of the flies have been killed.

In this test, for example, the following compound of the preparation examples shows, at a test concentration of 100 ppm, an efficacy of ≧80%: I-3-16.

### Example 8

Meloidogne test (MELGIN spray treatment)

| | |
|---|---|
| Solvent: | 80 parts by weight of acetone |

To produce a suitable preparation of active compound, 1 part by weight of active compound is mixed with the stated amount of water, and the concentrate is diluted with water to the desired concentration.

Vessels are filled with sand, solution of active compound, Meloidogyne incognita egg/larvae suspension and lettuce seeds. The lettuce seeds germinate and the plants develop. On the roots, galls are formed.

After the desired period of time, the nematicidal effect is determined in % by the gall formation. 100% means that no galls were found; 0% means that the number of galls on the treated plants corresponds to that of the untreated control.

In this test, for example, the following compounds of the preparation examples show, at a concentration of 20 ppm, an efficacy of ≧80%: I-3-7, I-3-41, I-3-42, I-3-46, I-3-59, I-3-61, I-3-64, I-3-65, I-3-66, I-3-70, I-3-71, I-3-78, I-3-80, I-3-82, I-3-96, I-3-101, I-3-115, I-3-116, I-3-117, I-3-118, I-3-120, I-3-127, I-3-131, I-3-133, I-3-135, I-3-136, I-3-137, I-3-138, I-3-163, I-3-164, I-3-166, I-3-198, I-3-200, I-3-262, I-3-298, I-3-311,1-3-321, I-3-331, I-3-334, I-3-369, I-3-407.

In this test, for example, the following compounds of the preparation examples show, at a concentration of 8 ppm, an efficacy of ≧80%: I-3-10, I-3-19, I-3-21, I-3-24.

In this test, for example, the following compounds of the preparation examples show, at a concentration of 4 ppm, an efficacy of ≧80%: I-3-1, I-3-29, I-3-30, I-3-99, I-3-193, I-3-194, I-3-227, I-3-230, I-3-275.

### Example 9

Nilaparvata lugens test (NILALU hydroponic treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

The preparation of active compound is pipetted into water. The stated concentration refers to the amount of active compound per volume unit of water (mg/l=ppm). The water is then infected with the brown planthopper (Nilaparvata lugens).

After the desired period of time, the effect in % is determined. 100% means that all planthoppers have been killed; 0% means that none of the planthoppers have been killed.

In this test, for example, the following compound of the preparation examples shows, at a concentration of 500 ppm, an efficacy of ≧80%: I-3-52.

### Example 10

Heliothis virescens test (HELIVI spray treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Soya bean leaves (Glycine max.) are sprayed with an active compound preparation of the desired concentration and, after they have dried, populated with eggs of the tobacco budworm (Heliothis virescens).

After the desired period of time, the effect in % is determined. 100% means that all eggs have been killed; 0% means that none of the eggs have been killed.

In this test, for example, the following compound of the preparation examples shows, at a concentration of 500 ppm, an efficacy of ≧80%: I-3-50.

### Example 11

Activity increase by ammonium/phosphonium salts in combination with penetrants

Myzus persicae test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration. If the addition of ammonium salts or ammonium salts and penetrants is required, these are in each case added in a concentration of 1000 ppm after dilution to the finished solution of the preparation.

Bell pepper plants (Capsicum annuum) which are heavily infested by the green peach aphid (Myzus persicaei) are treated by being sprayed with the active compound preparation at the desired concentration.

After the desired period of time, the kill in % is determined. 100% means that all animals have been killed; 0% means that none of the animals have been killed.

In this test, for example, the following compound of the preparation examples shows good efficacy: see table

TABLE

| | Concen- | | | | |
|---|---|---|---|---|---|
| Active | tration | Kill (%) | +AS | +RME | +AS + RME |
| compound | (ppm) | after 6 d | 1000 ppm | 1000 pm | 1000 ppm each |
| I-3-31 | 100 | 40 | 50 | 98 | 99 |
| | 20 | 0 | 0 | 0 | 70 |

### Example 12

Activity increase by ammonium/phosphonium salts in combination with penetrants

Aphis gossypii test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration. If the addition of ammonium salts or ammonium salts and penetrants is required, these are in each case added in a concentration of 1000 ppm after dilution to the finished solution of the preparation.

Cotton plants (Gossypium hirsutum) which are heavily infested by the cotton aphid (Aphis gossypii) are treated by being sprayed to runoff point with the active compound preparation at the desired concentration.

After the desired period of time, the kill in % is determined. 100% means that all aphids have been killed; 0% means that none of the aphids have been killed.

In this test, for example, the following compound of the preparation examples shows good efficacy: see table

TABLE

| | Concen- | | | | |
|---|---|---|---|---|---|
| Active | tration | Kill (%) | +AS | +RME | +AS + RME |
| compound | (ppm) | after 6 d | 1000 ppm | 1000 pm | 1000 ppm each |
| I-3-323 | 100 | 90 | 95 | 99 | 99 |
| | 20 | 10 | 10 | 75 | 90 |

1. A method for controlling animal pests comprising using at least one heterocyclic carboxylic acid derivative of the formula (I) 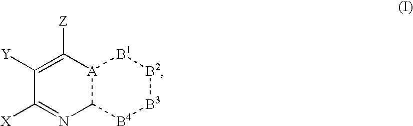
in which
A represents a nitrogen atom, in which case the bond in the pyrimidine ring is a single bond, or represents a carbon atom, in which case the bond in the pyridine ring is a double bond,
B¹ represents a nitrogen atom —N═ or represents the fragment 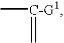
B² represents a nitrogen atom —N═ or represents the fragment 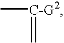
B³ represents a nitrogen atom —N═ or represents the fragment 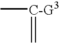
or represents a bond,
and
B⁴ represents the fragment 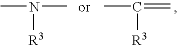
where the dashed bonds may be single bonds, double bonds or aromatic bonds;
X represents hydrogen, halogen, cyano, hydroxyl, alkyl, alkoxy, phenyl, alkylthio, alkylsulphinyl or alkylsulphonyl;
Y represents aryl, heterocyclyl, alkyl, alkenyl, alkynyl, cycloalkyl, cycloalkenyl, arylalkyl, halogen, an amino group, (C₁-C₈)-alkoxy, (C₁-C₈)-alkylthio, (C₆-C₁₀)-aryloxy, (C₆-C₁₀)-arylthio, heterocyclyloxy, (C₆-C₁₀)-aryl-(C₁-C₄)-alkoxy, (C₆-C₁₀)-aryl-(C₁-C₄)-alkylthio, heterocyclyl-(C₁-C₄)-alkoxy, heterocyclyl-(C₁-C₄)-alkylthio, C(S)OR⁸, C(O)SR⁸ or C(S)SR⁸;
Z represents hydroxyl, halogen or alkoxy, alkylthio, alkylsulphonyl or the group 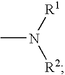
G¹, G² and G³ independently of one another represent hydrogen, halogen, alkyl or cycloalkyl, O—(C₁-C₄)-alkyl or S(O)₀₋₂(C₁-C₄)-alkyl;
L represents oxygen or sulphur;
R¹ represents hydrogen, alkyl, alkenyl, alkynyl, cycloalkyl or heterocyclyl, hydroxyl, alkoxy, amine, alkylamine or dialkylamine;
R² represents hydrogen or alkyl;
or
R¹ and R² together with the nitrogen atom to which they are attached represent a heterocyclic ring;
R³ represents 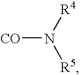
CO—OR⁶, CO—SR⁶, CS—OR⁶ or CS—SR⁶;
R⁴ represents hydrogen, alkyl, alkenyl or alkynyl, in each case saturated or unsaturated cycloalkyl which may optionally be interrupted by heteroatoms, phenyl, heteroaryl, arylalkyl or heteroarylalkyl or a cation such as, for example, a mono- or divalent metal atom or an ammonium ion;
R⁵ represents hydrogen, alkyl, alkoxy, alkenyl, alkenyloxy or alkynyl, represents the groups COR⁷, S(O)₁₋₂R⁷, cyano, COOR⁷, 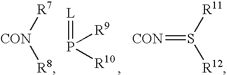
or saturated, partially or fully unsaturated or aromatic, 5- or 6-membered heterocyclyl which optionally contains one or up to three further heteroatoms selected from the group consisting of nitrogen, sulphur and oxygen atoms, where oxygen atoms must not be adjacent to one another;
or
R⁴ and R⁵ together with the nitrogen atom to which they are attached represent a saturated, unsaturated or aromatic ring which may optionally be interrupted by further heteroatoms;
R⁶ represents hydrogen, a cation, for example an ammonium ion, alkyl, alkenyl, alkynyl, cycloalkyl or cycloalkenyl which may optionally be interrupted by a heteroatom, cycloalkylalkyl or aryl, arylalkyl, heteroaryl or heteroarylalkyl;
R⁷ represents hydrogen, alkyl, alkenyl, alkynyl, represents cycloalkylalkyl, cycloalkyl or cycloalkenyl, aryl, heteroaryl, arylalkyl or heteroarylalkyl;
R⁸ represents hydrogen, alkyl, alkenyl, alkynyl, alkoxy or NH—R⁴; or
R⁴ and R⁷ together with the N—CO or N—S(O)₁₋₂ group to which they are attached form a 4- to 8-membered cycle which may contain one or more heteroatoms from the group consisting of sulphur, oxygen and nitrogen, where oxygen atoms must not be adjacent to one another; or
R⁷ and R⁸ together with the nitrogen atom to which they are attached represent an optionally substituted saturated or unsaturated ring which may optionally be interrupted by further heteroatoms;
R⁹ and R¹⁰ independently of one another represent alkyl, alkenyl, alkoxy, alkenyloxy, alkynyloxy, cycloalkoxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy or heteroarylalkylthio; or
R⁹ and R¹⁰ together with the phosphorus atom to which they are attached represent an optionally substituted 5- to 7-membered cycle which may be interrupted by one or two oxygen and/or sulphur atoms;
and
R¹¹ and R¹² independently of one another represent alkyl, alkenyl, alkynyl, phenyl or phenylalkyl;
or
of an agrochemically active salt thereof.

2. A method according to claim 1, where, if independently of one another,
X represents the radical alkyl, alkoxy, phenyl, alkylthio, alkylsulphinyl or alkylsulphonyl,
where the radicals mentioned above are unsubstituted or substituted; and/or
Y represents the radical aryl, heterocyclyl, alkyl, alkenyl, alkynyl, cycloalkyl, cycloalkenyl, arylalkyl, amino group, (C₁-C₈)-alkoxy, (C₁-C₈)-alkylthio, (C₆-C₁₀)-aryloxy, (C₆-C₁₀)-arylthio, heterocyclyloxy, (C₆-C₁₀)-aryl-(C₁-C₄)-alkoxy, (C₆-C₁₀)-aryl-(C₁-C₄)-alkylthio, heterocyclyl-(C₁-C₄)-alkoxy or heterocyclyl-(C₁-C₄)-alkylthio,
where the radicals mentioned above are unsubstituted or substituted; and/or
Y represents the radical alkyl, alkenyl, alkynyl, cycloalkyl or arylalkyl,
where the radicals mentioned above are unsubstituted or partially or fully halogenated and/or optionally carry one to three radicals R^{x}, where the R^{x} are identical or different and are selected from the group consisting of cyano, nitro, hydroxyl, C₁-C₆-alkyl, C₁-C₆-haloalkyl, C₃-C₆-cycloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylamino, di-C₁-C₆-alkylamino, C₂-C₆-alkenyl, C₂-C₆-alkenyloxy, C₂-C₆-alkynyl, C₃-C₆-alkynyloxy and optionally halogenated oxy-C₁-C₄-alkyl-C₁-C₄-alkenoxy, oxy-C₁-C₄-alkenyl-C₁-C₄-alkoxy and oxy-C₁-C₄-alkyl-C₁-C₄-alkyloxy; and/or
Y represents aryl,
where the radical mentioned above is unsubstituted or
mono- to tetrasubstituted by identical or different substituents from the group consisting of halogen, cyano, nitro, amino, hydroxyl, formyl, carboxy, carboxyalkyl, carbamoyl and thiocarb-amoyl;
in each case straight-chain or branched alkyl, alkoxy, alkylthio, alkylsulphinyl or alkylsulphonyl having in each case 1 to 6 carbon atoms;
in each case straight-chain or branched alkenyl, alkynyl, alkenyloxy or alkynyloxy having in each case 2 to 6 carbon atoms;
in each case straight-chain or branched haloalkyl, haloalkoxy, haloalkylthio, haloalkylsulphinyl or haloalkylsulphonyl having in each case 1 to 6 carbon atoms and 1 to 13 identical or different halogen atoms;
in each case straight-chain or branched haloalkenyl or haloalkenyloxy having in each case 2 to 6 carbon atoms and 1 to 11 identical or different halogen atoms;
in each case straight-chain or branched alkylamino, dialkylamino, alkylcarbonyl, alkylcarbonyl-oxy, alkoxycarbonyl, alkylsulphonyloxy, hydroximinoalkyl or alkoximinoalkyl having in each case 1 to 6 carbon atoms in the individual alkyl moieties;
cycloalkyl having 3 to 8 carbon atoms which is optionally mono- or disubstituted by fluorine, chlorine, C₁-C₃-alkyl or C₁-C₃-alkoxy; or
in each case 2,3- or 3,4-attached 1,3-propanediyl, 1,4-butanediyl, methylenedioxy (—O—CH₂—O—) or 1,2-ethylenedioxy (—O—CH₂—CH₂—O—), where these radicals may be mono- or polysubstituted by identical or different substituents from the group consisting of halogen, alkyl having 1 to 4 carbon atoms and haloalkyl having 1 to 4 carbon atoms and 1 to 9 identical or different halogen atoms; and/or
Y represents heterocyclyl,
where the radical mentioned above is unsubstituted or mono- or disubstituted by identical or different substituents from the group consisting of halogen, alkyl having 1 to 4 carbon atoms, alkoxy having 1 to 4 carbon atoms, alkylthio having 1 to 4 carbon atoms, haloalkoxy having 1 to 4 carbon atoms, haloalkylthio having 1 to 4 carbon atoms, hydroxyl, mercapto, cyano, nitro and cycloalkyl having 3 to 6 carbon atoms and carboxyalkyl; and/or
Z represents the radical alkoxy, alkylthio or alkylsulphonyl,
where the radicals mentioned above are unsubstituted or substituted, preferably unsubstituted or substituted by one to seven halogen atoms; and/or
G¹, G² and G³ independently of one another represent the radical alkyl, cycloalkyl O—(C₁-C₄)-alkyl or S(O)₀₋₂(C₁-C₄)-alkyl,
where the radicals mentioned above independently of one another are unsubstituted or substituted, preferably independently of one another unsubstituted or substituted by one or more halogen atoms; and/or
R¹ represents the radical alkyl, alkenyl, alkynyl, cycloalkyl, heterocyclyl, alkoxy, alkylamine or dialkylamine,
where the radicals mentioned above are unsubstituted or substituted; and/or
R¹ represents alkyl,
where the radical mentioned above is unsubstituted or mono- to pentasubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- and dialkylamino having in each case 1 to 4 carbon atoms; and/or
R¹ represents alkenyl,
where the radical mentioned above is unsubstituted or mono- to trisubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- and dialkylamino having in each case 1 to 4 carbon atoms; and/or
R¹ represents alkynyl,
where the radical mentioned above is unsubstituted or mono- to trisubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- and dialkylamino having in each case 1 to 4 carbon atoms; and/or
R¹ represents cycloalkyl,
where the radical mentioned above is unsubstituted or mono- to trisubstituted by identical or different substituents from the group consisting of halogen, alkyl and haloalkyl having in each case 1 to 4 carbon atoms; and/or
R¹ represents heterocyclyl,
where the radical mentioned above is unsubstituted or mono- or polysubstituted by identical or different substituents from the group consisting of halogen, alkyl having 1 to 4 carbon atoms, alkoxy having 1 to 4 carbon atoms, cyano, nitro, cycloalkyl having 3 to 6 carbon atoms, hydroxyl, alkoxy, alkenyloxy, alkynyloxy having 1 to 6 carbon atoms and mercapto; and/or
R¹ and R² together with the nitrogen atom to which they are attached represent a heterocyclic ring,
where the heterocyclic ring mentioned above is unsubstituted or substituted, by identical or different substituents from the group consisting of fluorine, chlorine, bromine, alkyl having 1 to 4 carbon atoms, haloalkyl having 1 to 4 carbon atoms and 1 to 9 fluorine and/or chlorine atoms, hydroxyl, alkoxy having 1 to 4 carbon atoms, haloalkoxy having 1 to 4 carbon atoms and 1 to 9 fluorine and/or chlorine atoms, mercapto, thioalkyl having 1 to 4 carbon atoms and haloalkylthio having 1 to 4 carbon atoms and 1 to 9 fluorine and/or chlorine atoms; and/or
R⁴ represents the radical alkyl, alkenyl, alkynyl, saturated or unsaturated cycloalkyl which may optionally be interrupted by heteroatoms, phenyl, heteroaryl, arylalkyl, heteroarylalkyl or an ammonium ion,
where the radicals mentioned above are unsubstituted or substituted, or R⁴ is selected from the group consisting of alkylammonium, in particular mono-(C₁-C₁₀)-alkylammonium, di-(C₁-C₁₀)-alkylammonium, tri-(C₁-C₁₀)-alkylammonium and tetra-(C₁-C₁₀)-alkylammonium, where the alkyl radicals of the ammonium ions may be substituted by aryl or hydroxyl, alkyl, alkenyl and alkynyl, each of which is mono- or polysubstituted by identical or different substituents from the group consisting of fluorine, chlorine, C₁-C₄-alkoxy, C₁-C₄-alkylthio, cyano, CO₂—H and CO—O—C₁-C₄-alkyl, C₃-C₈-cycloalkyl and C₅-C₈-cycloalkenyl, each of which is mono- or polysubstituted by identical or different substituents from the group consisting of fluorine, chlorine, C₁-C₄-alkyl, C₁-C₄-haloalkyl, C₁-C₄-alkoxy, CN, CO₂H and CO—O—C₁-C₄-alkyl and each of which may optionally be interrupted by heteroatoms, phenyl, heteroaryl, arylalkyl and heteroarylalkyl, each of which is mono- to trisubstituted by identical or different substituents from the group consisting of fluorine, chlorine, bromine C₁-C₄-alkyl, C₁-C₄-haloalkyl, C₁-C₄-alkoxy, C₁-C₄-haloalkoxy, nitro and cyano; and/or
R⁵ represents the radical alkyl, alkoxy, alkenyl, alkenyloxy, alkynyl or saturated, partially or fully unsaturated or aromatic 5- or 6-membered heterocyclyl which optionally contains one or up to three further heteroatoms selected from the group consisting of nitrogen, sulphur and oxygen atoms, where oxygen atoms must not be adjacent to one another,
where the radicals mentioned above are unsubstituted or substituted, or R⁵ is selected from the group consisting of alkyl, alkoxy, alkenyl, alkenyloxy, alkynyl, each of which is mono- or polysubstituted by identical or different substituents from the group consisting of fluorine and chlorine and heterocyclyl which is mono- or disubstituted by identical or different substituents from the group consisting of fluorine, chlorine, bromine, C₁-C₄-alkyl, C₁-C₄-alkoxy, cyano and nitro; and/or
R⁴ and R⁵ together with the nitrogen atom to which they are attached represent a saturated, unsaturated or aromatic ring which may optionally be interrupted by further heteroatoms,
where the ring mentioned above is unsubstituted or substituted, or mono- or polysubstituted by identical or different substituents from the group consisting of C₁-C₄-alkyl; and/or
R⁶ represents an ammonium ion, alkyl, alkenyl, alkynyl, cycloalkyl or cycloalkenyl which may optionally be interrupted by a heteroatom, cycloalkylalkyl, aryl, arylalkyl, heteroaryl or heteroarylalkyl,
where the radicals mentioned above are unsubstituted or substituted, or R⁶ is selected from the group consisting of alkylammonium, in particular mono-(C₁-C₁₀)-alkylammonium, di-(C₁-C₁₀)-alkylammonium, tri-(C₁-C₁₀)-alkylammonium and tetra-(C₁-C₁₀)-alkylammonium, where the alkyl radicals of the ammonium ions may be substituted by identical or different substituents from the group consisting of aryl and hydroxyl, alkyl, alkenyl and alkynyl, each of which is mono- or polysubstituted by identical of different substituents from the group consisting of fluorine, chlorine, C₁-C₄-alkoxy, C₁-C₄-alkylthio, cyano, CO₂—H and CO—O—C₁-C₄-alkyl, cycloalkyl, cycloalkylalkyl and cycloalkenyl, each of which is mono- or polysubstituted by identical of different substituents from the group consisting of fluorine, chlorine, C₁-C₄-alkyl, C₁-C₄-haloalkyl, C₁-C₄-alkoxy, CN, CO₂H and CO—O—C₁-C₄-alkyl and each of which may optionally be interrupted by a heteroatom, and aryl, arylalkyl, heteroaryl and heteroarylalkyl, each of which is mono- to trisubstituted by identical of different substituents from the group consisting of fluorine, chlorine, bromine, C₁-C₄-alkyl, C₁-C₄-haloalkyl, C₁-C₄-alkoxy, C₁-C₄-haloalkoxy, nitro and cyano; and/or
R⁷ represents the radicals alkyl, alkenyl, alkynyl, cycloalkylalkyl, cycloalkyl, cycloalkenyl, aryl, heteroaryl, arylalkyl or heteroarylalkyl,
where the radicals mentioned above are unsubstituted or substituted, preferably unsubstituted, or R⁷ is selected from the group consisting of alkyl, alkenyl and alkynyl, each of which is mono- or polysubstituted by identical or different substituents from the group consisting of fluorine, chlorine, C₁-C₄-alkoxy, C₁-C₄-alkylthio, cyano, CO₂-H and CO—O—C₁-C₄-alkyl, cycloalkyl, cycloalkylalkyl and cycloalkenyl, each of which is mono- or polysubstituted by identical or different substituents from the group consisting of fluorine, chlorine, C₁-C₄-alkyl, C₁-C₄-haloalkyl, C₁-C₄-alkoxy, CN, CO₂H and CO—O—C₁-C₄-alkyl, aryl, heteroaryl, arylalkyl and heteroarylalkyl, each of which is mono- to trisubstituted by identical or different substituents from the group consisting of fluorine, chlorine, bromine, C₁-C₄-alkyl, C₁-C₄-haloalkyl, C₁-C₄-alkoxy, C₁-C₄-haloalkoxy, nitro and cyano; and/or
R⁸ represents the radicals alkyl, alkenyl, alkynyl or alkoxy,
where the radicals mentioned above are unsubstituted or substituted, or R⁸ is selected from the group consisting of alkyl, alkenyl, alkynyl and alkoxy, each of which is mono- or polysubstituted by identical or different substituents from the group consisting of fluorine and chlorine; and/or
R⁷ and R⁸ together with the nitrogen atom to which they are attached represent a saturated or unsaturated ring which may optionally be interrupted by further heteroatoms,
where the ring mentioned above is unsubstituted or substituted, or mono- or polysubstituted by identical or different substituents from the group consisting of C₁-C₄-alkyl; and/or
R⁹ and R¹⁰ independently of one another represent alkyl, alkenyl, alkoxy, alkenyloxy, alkynyloxy, cycloalkoxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy or heteroarylalkylthio,
where the radicals mentioned above are unsubstituted or substituted, or R⁹ and R¹⁰ independently of one another are selected from the group consisting of alkyl, alkenyl, alkoxy, alkenyloxy, alkynyloxy, alkylthio and alkenylthio, each of which is mono- or polysubstituted by identical or different substituents from the group consisting of fluorine and chlorine, cycloalkoxy, cycloalkenyloxy and cycloalkylalkoxy, each of which is mono- or disubstituted by identical or different substituents from the group consisting of fluorine, chlorine, C₁-C₄-alkyl, C₁-C₄-haloalkyl and C₁-C₄-alkoxy, phenoxy, phenylthio, benzyloxy, benzylthio, heteroaryloxy, heteroarylthio, heteroarylalkoxy and heteroarylalkylthio, each of which is mono- to trisubstituted by identical or different substituents from the group consisting of fluorine, chlorine, bromine, C₁-C₆-alkyl-C₁-C₄-haloalkyl, C₁-C₆-alkoxy, C₁-C₄-haloalkoxy, C₁-C₆-alkylthio, cyano and nitro; and/or
R⁹ and R¹⁰ together with the phosphorus atom to which they are attached represent a 5- to 7-membered cycle which may be interrupted by one or two oxygen and/or sulphur atoms,
where the cycle mentioned above is unsubstituted or substituted, or mono- to trisubstituted by identical or different substituents from the group consisting of fluorine, chlorine, C₁-C₄-alkyl, C₁-C₄-alkoxy and C₁-C₄-haloalkyl; and/or
R¹¹ and R¹² independently of one another represent the radicals alkyl, alkenyl, alkynyl, phenyl or phenylalkyl,
where the radicals mentioned above are unsubstituted or substituted, or R¹¹ and R¹² independently of one another are selected from the group consisting of alkyl, alkenyl and alkynyl, each of which is mono- to trisubstituted by identical or different substituents from the group consisting of fluorine and chlorine, and phenyl and phenylalkyl, each of which is mono- or disubstituted by identical or different substituents from the group consisting of fluorine, chlorine, bromine, C₁-C₄-alkyl, C₁-C₄ haloalkyl, C₁-C₄-alkoxy, C₁-C₄-haloalkoxy, cyano and nitro.

3. A method according to claim 1, where
A represents a nitrogen atom, in which case the bond in the pyrimidine ring is a single bond, or represents a carbon atom, in which case the bond in the pyridine ring is a double bond,
B¹ represents a nitrogen atom —N═ or represents the fragment 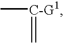
B² represents a nitrogen atom —N═ or represents the fragment 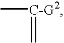
B³ represents a nitrogen atom —N═ or represents the fragment 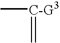
or represents a bond,
and
B⁴ represents the fragment 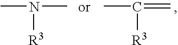
where the dashed bonds may be single bonds, double bonds or aromatic bonds;
X represents hydrogen, fluorine, chlorine, bromine, cyano, hydroxyl, alkoxy having 1 to 4 carbon atoms or alkylthio having 1 to 4 carbon atoms, C₁-C₆-alkyl or C₁-C₄-haloalkyl;
Y represents C₁-C₁₀-alkyl, C₂-C₁₀-alkenyl, C₂-C₁₀-alkynyl, C₃-C₈-cycloalkyl, phenyl-C₁-C₁₀-alkyl, where Y is unsubstituted or partially or fully halogenated and/or optionally carries one to three radicals R^{x}, or represents C₁-C₁₀-haloalkyl, which optionally carries one to three radicals R^{x}, where the R^{x} are identical or different and selected from the group consisting of cyano, nitro, hydroxyl, C₁-C₆-alkyl, C₁-C₆-haloalkyl, C₃-C₆-cycloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylamino, di-C₁-C₆-alkylamino, C₂-C₆-alkenyl, C₂-C₆-alkenyloxy, C₂-C₆-alkynyl, C₃-C₆-alkynyloxy and optionally halogenated oxy-C₁-C₄-alkyl-C₁-C₄-alkenoxy, oxy-C₁-C₄-alkenyl-C₁-C₄-alkoxy and oxy-C₁-C₄-alkyl-C₁-C₄-alkyloxy;
or
Y represents phenyl which may optionally be mono- to tetrasubstituted by identical or different substituents selected from the group consisting of halogen, cyano, nitro, amino, hydroxyl, formyl, carboxy, carboxyalkyl, carbamoyl, thiocarbamoyl;
in each case straight-chain or branched alkyl, alkoxy, alkylthio, alkylsulphinyl or alkylsulphonyl having in each case 1 to 6 carbon atoms;
in each case straight-chain or branched alkenyl, alkynyl, alkynyloxy or alkenyloxy having in each case 2 to 6 carbon atoms;
in each case straight-chain or branched haloalkyl, haloalkoxy, haloalkylthio, haloalkylsulphinyl or haloalkylsulphonyl having in each case 1 to 6 carbon atoms and 1 to 13 identical or different halogen atoms;
in each case straight-chain or branched haloalkenyl or haloalkenyloxy having in each case 2 to 6 carbon atoms and 1 to 11 identical or different halogen atoms;
in each case straight-chain or branched alkylamino, dialkylamino, alkylcarbonyl, alkylcarbonyl-oxy, alkoxycarbonyl, alkylsulphonyloxy, hydroximinoalkyl or alkoximinoalkyl having in each case 1 to 6 carbon atoms in the individual alkyl moieties;
cycloalkyl having 3 to 8 carbon atoms which is optionally mono- or disubstituted by fluorine, chlorine, C₁-C₃-alkyl or C₁-C₃-alkoxy; or
2,3- or 3,4-attached 1,3-propanediyl, 1,4-butanediyl, methylenedioxy (—O—CH₂—O—) or 1,2-ethylenedioxy (—O—CH₂—CH₂—O—), where these radicals may be mono- or polysubstituted by identical or different substituents from the group consisting of halogen, alkyl having 1 to 4 carbon atoms and haloalkyl having 1 to 4 carbon atoms and 1 to 9 identical or different halogen atoms;
or
Y represents saturated or fully or partially unsaturated or aromatic heterocyclyl having 3 to 8 ring members and 1 to 3 heteroatoms from the group consisting of nitrogen, oxygen and sulphur, where the heterocyclyl may be mono- or disubstituted by halogen, alkyl having 1 to 4 carbon atoms, alkoxy having 1 to 4 carbon atoms, alkylthio having 1 to 4 carbon atoms, haloalkoxy having 1 to 4 carbon atoms, haloalkylthio having 1 to 4 carbon atoms, hydroxyl, mercapto, cyano, nitro and/or cycloalkyl having 3 to 6 carbon atoms and/or carboxyalkyl;
Z represents hydroxyl, chlorine, bromine, straight-chain or branched C₁-C₆-alkoxy, C₁-C₆-alkylthio or C₁-C₆-alkylsulphonyl, each of which is optionally substituted by 1 to 7 halogen atoms, or the group 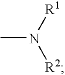
G¹, G² and G³ independently of one another represent hydrogen, halogen, C₁-C₄-alkyl which is optionally substituted by one or more halogen atoms, C₃-C₆-cycloalkyl which is optionally substituted by one or more halogen atoms, SCH₃, SC₂H₅, SOCH₃, SOC₂H₅, SO₂CH₃, SO₂C₂H₅, OCH₃ or OC₂H₅;
L represents oxygen or sulphur;
R¹ represents hydrogen, alkyl having 1 to 10 carbon atoms which is unsubstituted or mono- to pentasubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms;
or
R¹ represents alkenyl having 3 to 10 carbon atoms which is unsubstituted or mono- to trisubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms;
or
R¹ represents alkynyl having 3 to 10 carbon atoms which is unsubstituted or mono- to trisubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms;
or
R¹ represents cycloalkyl having 3 to 10 carbon atoms which is unsubstituted or mono- to trisubstituted by identical or different substituents from the group consisting of halogen and alkyl, haloalkyl having in each case 1 to 4 carbon atoms;
or
R¹ represents saturated or unsaturated heterocyclyl having 3 to 10 ring members and 1 to 3 heteroatoms, such as nitrogen, oxygen and/or sulphur, where the heterocyclyl is unsubstituted or mono- or polysubstituted by halogen, alkyl having 1 to 4 carbon atoms, alkoxy having 1 to 4 carbon atoms, cyano, nitro, cycloalkyl having 3 to 6 carbon atoms, hydroxyl, alkoxy, alkenyloxy, alkynyloxy having 1 to 6 carbon atoms or mercapto;
R² represents hydrogen or alkyl having 1 to 6 carbon atoms;
or
R¹ and R² together with the nitrogen atom to which they are attached represent a saturated or unsaturated heterocyclic ring having 3 to 8 ring members, where the heterocycle optionally contains a further nitrogen, oxygen or sulphur atom as ring member and where the heterocycle may be unsubstituted or up to trisubstituted by fluorine, chlorine, bromine, alkyl having 1 to 4 carbon atoms, haloalkyl having 1 to 4 carbon atoms and 1 to 9 fluorine and/or chlorine atoms, hydroxyl, alkoxy having 1 to 4 carbon atoms, haloalkoxy having 1 to 4 carbon atoms and 1 to 9 fluorine and/or chlorine atoms, mercapto, thioalkyl having 1 to 4 carbon atoms and/or haloalkylthio having 1 to 4 carbon atoms and 1 to 9 fluorine and/or chlorine atoms;
R³ represents 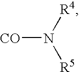
CO—OR⁶, CO—SR⁶, CS—OR⁶ or CS—SR⁶;
R⁴ represents hydrogen, cations from the group of the alkali metals or alkaline earth metals or ammonium ions NH₄, mono-(C₁-C₁₀)-alkylammonium, di-(C₁-C₁₀)-alkylammonium, tri-(C₁-C₁₀)-alkylammonium, tetra-(C₁-C₁₀)-alkylammonium, where the alkyl radicals of the ammonium ions may be substituted by aryl or hydroxyl, or cholinium,
represents C₁-C₁₀-alkyl, C₃-C₁₀-alkenyl or C₃-C₁₀-alkynyl, each of which is optionally mono- or polysubstituted by fluorine, chlorine, C₁-C₄-alkoxy, C₁-C₄-alkylthio, cyano, CO₂-H or CO—O—C₁-C₄-alkyl, represents C₃-C₈-cycloalkyl or C₅-C₈-cycloalkenyl, each of which is optionally mono- or polysubstituted by fluorine, chlorine, C₁-C₄-alkyl, C₁-C₄-haloalkyl, C₁-C₄-alkoxy, CN, CO₂H or CO—O—C₁-C₄-alkyl and each of which may optionally be interrupted by an oxygen or sulphur atom,
represents phenyl, pyridyl, thiazolyl, pyrimidyl, benzyl, phenethyl, pyridinylmethyl or thiazolylmethyl, each of which is optionally mono- to trisubstituted by fluorine, chlorine, bromine, C₁-C₄-alkyl, C₁-C₄-halo alkyl, C₁-C₄-alkoxy, C₁-C₄-haloalkoxy, nitro or cyano;
R⁵ represents hydrogen, represents C₁-C₁₀-alkyl, C₁-C₁₀-alkoxy, C₃-C₁₀-alkenyl, C₃-C₁₀-alkenyloxy or C₃-C₁₀-alkynyl, each of which is optionally mono- or polysubstituted by fluorine and/or chlorine; represents the groups CO—R⁷, S(O)₁₋₂R⁷, cyano, COOR⁷, 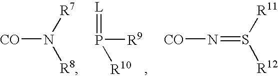
or represents pyridine, pyrimidine, triazine, thiazole or pyrazole, each of which is optionally mono- or disubstituted by fluorine, chlorine, bromine, C₁-C₄-alkyl, C₁-C₄-alkoxy, cyano or nitro;
or
R⁴ and R⁵ together with the nitrogen atom to which they are attached represent a saturated or unsaturated 5- or 6-membered ring which is optionally mono- or polysubstituted by C₁-C₄-alkyl and which may optionally be interrupted by oxygen or sulphur;
R⁶ represents hydrogen, cations from the group of the alkali metals or alkaline earth metals or ammonium ions NH₄, mono-(C₁-C₁₀)-alkylammonium, di-(C₁-C₁₀)-alkylammonium, tri-(C₁-C₁₀)-alkylammonium, tetra-(C₁-C₁₀)-alkylammonium, where the alkyl radicals of the ammonium ions may be substituted by aryl or hydroxyl, or cholinium,
represents C₁-C₁₀-alkyl, C₃-C₁₀-alkenyl or C₃-C₁₀-alkynyl, each of which is optionally mono- or polysubstituted by fluorine, chlorine, C₁-C₄-alkoxy, C₁-C₄-alkylthio, cyano, CO₂-H or CO—O—C₁-C₄-alkyl, represents C₃-C₈-cycloalkyl, C₃-C₆-cycloalkyl-C₁-C₂-alkyl or C₅-C₈-cycloalkenyl, each of which is optionally mono- or polysubstituted by fluorine, chlorine, C₁-C₄-alkyl, C₁-C₄-haloalkyl, C₁-C₄-alkoxy, CN, CO₂H or CO—O—C₁-C₄-alkyl and each of which may optionally be interrupted by an oxygen or sulphur atom,
represents phenyl, pyridyl, thiazolyl, pyrimidyl, benzyl, phenethyl, pyridinylmethyl or thiazolylmethyl, each of which is optionally mono- to trisubstituted by fluorine, chlorine, bromine, C₁-C₄-alkyl, C₁-C₄-haloalkyl, C₁-C₄-alkoxy, C₁-C₄-haloalkoxy, nitro or cyano;
R⁷ represents hydrogen, represents C₁-C₁₀-alkyl, C₃-C₁₀-alkenyl or C₃-C₁₀-alkynyl, each of which is optionally mono- or polysubstituted by fluorine, chlorine, C₁-C₄-alkoxy, C₁-C₄-alkylthio, cyano, CO₂—H or CO—O—C₁-C₄-alkyl, represents C₃-C₈-cycloalkyl, C₃-C₆-cycloalkyl-C₁-C₂-alkyl or C₅-C₈-cycloalkenyl, each of which is optionally mono- or polysubstituted by fluorine, chlorine, C₁-C₄-alkyl, C₁-C₄-haloalkyl, C₁-C₄-alkoxy, CN, CO₂H or CO—O—C₁-C₄-alkyl and each of which may optionally be interrupted by an oxygen or sulphur atom,
represents phenyl, pyridyl, thiazolyl, pyrimidyl, benzyl, phenethyl, pyridinylmethyl or thiazolylmethyl, each of which is optionally mono- to trisubstituted by fluorine, chlorine, bromine, C₁-C₄-alkyl, C₁-C₄-haloalkyl, C₁-C₄-alkoxy, C₁-C₄-haloalkoxy, nitro or cyano;
R⁸ represents hydrogen, represents C₁-C₆-alkyl, C₃-C₆-alkenyl, C₃-C₆-alkynyl or C₁-C₆-alkoxy, each of which is optionally mono- or polysubstituted by fluorine and/or chlorine;
or
R⁴ and R⁷ together with the N—CO or N—S(O)₁₋₂ group to which they are attached form a 4- to 8-membered cycle which may contain one or more heteroatoms from the group consisting of sulphur, oxygen and nitrogen, where oxygen atoms must not be adjacent to one another;
or
R⁷ and R⁸ together with the nitrogen atom to which they are attached represent a saturated or unsaturated 5- or 6-membered ring which is optionally mono- or polysubstituted by C₁-C₄-alkyl and which may optionally be interrupted by oxygen or sulphur;
R⁹ and R¹⁰ independently of one another represent C₁-C₈-alkyl, C₃-C₈-alkenyl, C₁-C₈-alkoxy, C₃-C₈-alkenyloxy, C₃-C₈-alkynyloxy, C₁-C₆-alkylthio, C₃-C₆-alkenylthio, each of which is optionally mono- or polysubstituted by fluorine and/or chlorine, C₃-C₈-cycloalkoxy, C₄-C₈-cycloalkenyloxy or C₃-C₈-cycloalkyl-C₁-C₂-alkoxy, each of which is optionally mono- or disubstituted by fluorine, chlorine, C₁-C₄-alkyl, C₁-C₄ haloalkyl, C₁-C₄-alkoxy, represent phenoxy, phenylthio, benzyloxy, benzylthio, pyridinyloxy, pyrazolyloxy, pyridinylthio, pyrimidylthio, thiazolylthio, pyridyl-C₁-C₂-alkoxy, pyrimidyl-C₁-C₂-alkyloxy, thiazolyl-C₁-C₂-alkyloxy, pyridyl-C₁-C₂-alkylthio, thiazolyl-C₁-C₂-alkylthio or pyrimidyl-C₁-C₂-alkylthio, each of which is optionally mono- to trisubstituted by fluorine, chlorine, bromine, C₁-C₆-alkyl-C₁-C₄-haloalkyl, C₁-C₆-alkoxy, C₁-C₄-haloalkoxy, C₁-C₆-alkylthio, cyano or nitro;
or
R⁹ and R¹⁰ together with the phosphorus atom to which they are attached represent a five- to seven-membered cycle which is optionally mono- to trisubstituted by fluorine, chlorine, C₁-C₄-alkyl, C₁-C₄-alkoxy or C₁-C₄-haloalkyl and which may be interrupted by one or two oxygen and/or sulphur atoms;
and
R¹¹ and R¹² independently of one another represent C₁-C₈-alkyl, C₃-C₈-alkenyl or C₃-C₈-alkynyl, each of which is optionally mono- to trisubstituted by fluorine or chlorine, represent phenyl or benzyl, each of which is optionally mono- or disubstituted by fluorine, chlorine, bromine, C₁-C₄-alkyl, C₁-C₄ haloalkyl, C₁-C₄-alkoxy, C₁-C₄-haloalkoxy, cyano or nitro.

4. A method according to claim 1, where
A represents a nitrogen atom, in which case the bond in the pyrimidine ring is a single bond, or represents a carbon atom, in which case the bond in the pyridine ring is a double bond,
B¹ represents a nitrogen atom —N═ or represents the fragment 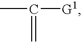
B² represents a nitrogen atom —N═ or represents the fragment 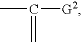
B³ represents a nitrogen atom —N═ or represents the fragment 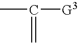
or represents a bond,
and
B⁴ represents the fragment 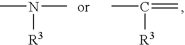
where the dashed bonds may be single bonds, double bonds or aromatic bonds;
X represents hydrogen, fluorine, chlorine, cyano, C₁-C₄-alkyl or C₁-C₂-haloalkyl which may be substituted by one to five fluorine and/or chlorine atoms;
Y represents C₁-C₈-alkyl, C₂-C₆-alkenyl, C₂-C₆-alkynyl or C₃-C₈-cycloalkyl, each of which is optionally substituted by one to five fluorine- and/or chlorine atoms;
or
Y represents phenyl which may optionally be mono- to trisubstituted by identical or different substituents from the group consisting of fluorine, chlorine, bromine, iodine, cyano, nitro, formyl, methyl, ethyl, n- or i-propyl, n-, s- or t-butyl, vinyl, 1-propenyl, ethynyl, 1-propynyl, allyl, propargyl, methoxy, ethoxy, n- or i-propoxy, methylthio, ethylthio, n- or i-propylthio, methylsulphinyl, ethylsulphinyl, methylsulphonyl, ethylsulphonyl, allyloxy, pro-pargyloxy, trifluoromethyl, trifluoroethyl, difluoromethoxy, trifluoromethoxy, difluorochloro-methoxy, trifluoroethoxy, difluoromethylthio, difluorochloromethylthio, trifluoromethylthio, trifluoromethylsulphinyl, trifluoromethylsulphonyl, trichloroethynyloxy, trifluoroethynyloxy, chloroallyloxy, iodopropargyloxy, methylamino, ethylamino, n- or i-propylamino, dimethyl-amino, diethylamino, acetyl, propionyl, acetyloxy, methoxycarbonyl, ethoxycarbonyl, hydrox-iminomethyl, hydroximinoethyl, methoximinomethyl, ethoximinomethyl, methoximinoethyl, ethoximinoethyl, cyclopropyl, cyclobutyl, cyclopentyl and cyclohexyl; or by
2,3- or 3,4-attached 1,3-propanediyl, 1,4-butanediyl, methylenedioxy (—O—CH₂—O—) or 1,2-ethylenedioxy (—O—CH₂—CH₂—O—), where these radicals may be mono- or polysubstituted by identical or different substituents from the group consisting of fluorine, chlorine, methyl, ethyl, n-propyl, i-propyl, trifluoromethyl, carboxyl and carboxymethyl;
or
Y represents pyridyl which is attached in the 2- or 4-position and may be mono- to tetrasubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluor-omethyl, carboxyl and carboxymethyl; or
thiazolyl which is attached in the 2-, 4- or 5-position and may be mono- or disubstituted by identical or different substituents from the group consisting of fluorine, chlorine, bromine, cyano, nitro, hydroxyl, mercapto, methyl, ethyl, methoxy, methylthio, hydroximinomethyl, hydroximinoethyl, methoximinomethyl, methoximinoethyl, trifluoromethyl, carboxyl and carboxymethyl; or
pyrimidyl which is attached in the 2- or 4-position and may be mono- to trisubstituted by identical or different substituents from the group consisting of fluorine, chlorine, bromine, cyano, nitro, hydroxyl, mercapto, methyl, ethyl, methoxy, methylthio, hydroximinomethyl, hydroximinoethyl, methoximinomethyl, methoximinoethyl, trifluoromethyl, carboxyl and carboxymethyl; or
thienyl which is attached in the 2- or 4-position and may be mono- to trisubstituted by identical or different substituents from the group consisting of fluorine, chlorine, bromine, cyano, nitro, hydroxyl, mercapto, methyl, ethyl, methoxy, methylthio, hydroximinomethyl, hydroximinoethyl, methoximinomethyl, methoximinoethyl, trifluoromethyl, carboxyl and carboxymethyl;
Z represents chlorine, bromine, represents straight-chain or branched C₁-C₄-alkoxy, C₁-C₄-alkylthio, each of which is optionally substituted by 1 to 3 halogen atoms, or represents the group 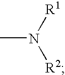
G¹, G² and G³ independently of one another represent hydrogen, halogen, (C₁-C₄)-alkyl which is optionally substituted by one or more halogen atoms, cyclopropyl which is optionally substituted by one or more halogen atoms; SCH₃, SC₂H₅, SOCH₃, SOC₂H₅, SO₂CH₃, SO₂C₂H₅, OCH₃ or OC₂H₅;
L represents oxygen or sulphur;
R¹ represents hydrogen, alkyl having 1 to 8 carbon atoms which is unsubstituted or mono- to trisubstituted by identical or different substituents from the group consisting of fluorine, chlorine, cyano, alkoxy having 1 to 3 carbon atoms, cycloalkyl having 3 to 6 carbon atoms, alkylthio having 1 to 3 carbon atoms;
or
R¹ represents alkenyl having 3 to 8 carbon atoms which is unsubstituted or mono- to trisubstituted by identical or different substituents from the group consisting of fluorine, chlorine, cyano, alkoxy having 1 to 3 carbon atoms, cycloalkyl having 3 to 6 carbon atoms, alkylthio having 1 to 3 carbon atoms;
or
R¹ represents alkynyl having 3 to 8 carbon atoms which is unsubstituted or mono- to trisubstituted by identical or different substituents from the group consisting of fluorine, chlorine, cyano, alkoxy having 1 to 3 carbon atoms, cycloalkyl having 3 to 6 carbon atoms, alkylthio having 1 to 3 carbon atoms;
or
R¹ represents cycloalkyl having 3 to 8 carbon atoms which is unsubstituted or mono- to trisubstituted by identical or different substituents from the group consisting of fluorine, chlorine and alkyl, haloalkyl, alkoxy having in each case 1 to 2 carbon atoms;
or
R¹ represents saturated or unsaturated heterocyclyl having 3 to 8 ring members and 1 to 2 heteroatoms, such as nitrogen, oxygen and/or sulphur, where the heterocyclyl is unsubstituted or mono- or disubstituted by hydroxyl, fluorine, chlorine, alkyl having 1 to 3 carbon atoms, alkoxy having 1 to 3 carbon atoms, cyano, nitro or cycloalkyl having 3 to 6 carbon atoms;
R² represents hydrogen or alkyl having 1 to 6 carbon atoms;
or
R¹ and R² together with the nitrogen atom to which they are attached represent a saturated or unsaturated heterocyclic ring having 3 to 6 ring members, where the heterocycle optionally contains a further nitrogen, oxygen or sulphur atom as ring member and where the heterocycle may be unsubstituted or up to trisubstituted by fluorine, chlorine, bromine, alkyl having 1 to 3 carbon atoms, haloalkyl having 1 to 3 carbon atoms and 1 to 5 fluorine and/or chlorine atoms, alkoxy having 1 to 3 carbon atoms, haloalkoxy having 1 to 3 carbon atoms and 1 to 5 fluorine and/or chlorine atoms, thioalkyl having 1 to 3 carbon atoms and/or haloalkylthio having 1 to 3 carbon atoms and 1 to 5 fluorine and/or chlorine atoms;
R³ represents 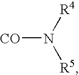
CO—OR⁶, CO—SR⁶, CS—OR⁶ or CS—SR⁶;
R⁴ represents hydrogen, Na⁺, K⁺, ½ Ca²⁺, ½ Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺, N(CH₃)₃—H₂—C₆H₅⁺, 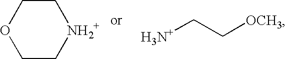
represents C₁-C₈-alkyl, C₃-C₈-alkenyl or C₃-C₈-alkynyl, each of which is optionally mono- to trisubstituted by fluorine, chlorine, C₁-C₃-alkoxy, C₁-C₃-alkylthio, cyano, CO₂H or CO—O—C₁-C₃-alkyl, represents C₃-C₆-cycloalkyl, C₃-C₆-cycloalkylmethyl or C₅-C₆-cycloalkenyl, each of which is optionally mono- to trisubstituted by fluorine, chlorine, C₁-C₃-alkyl, C₁-C₂-haloalkyl, C₁-C₃-alkoxy, CN, CO₂H or CO—O—C₁-C₃-alkyl and each of which may optionally be interrupted by an oxygen or sulphur atom,
represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, C₁-C₃-alkyl, C₁-C₃-haloalkyl, C₁-C₃-alkoxy, C₁-C₃-haloalkoxy, nitro or cyano;
R⁵ represents hydrogen, represents C₁-C₈-alkyl, C₁-C₈-alkoxy, C₃-C₈-alkenyl, C₃-C₈-alkenyloxy or C₃-C₈-alkynyl, each of which is optionally mono- to trisubstituted by fluorine and/or chlorine; represents the groups COR⁷, S(O)₁₋₂R⁷, cyano, COOR⁷, 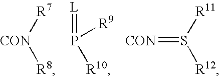
or represents pyridine, pyrimidine, triazine, thiazole, pyrazole, oxazole or triazole, each of which is optionally mono- or disubstituted by fluorine, chlorine, bromine, C₁-C₃-alkyl, C₁-C₃-alkoxy, cyano, hydroxyl or nitro;
or
R⁴ and R⁵ together with the nitrogen atom to which they are attached represent a saturated or unsaturated 5- or 6-membered ring which is optionally mono- or disubstituted by C₁-C₂-alkyl and which may optionally be interrupted by oxygen or sulphur;
R⁶ represents hydrogen, Na⁺, K⁺, ½ Ca²⁺, ½ Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺ or N(CH₃)₃—H₂—C₆H₅⁺,
represents C₁-C₈-alkyl, C₁-C₈-alkenyl or C₁-C₈-alkynyl, each of which is optionally mono- to trisubstituted by fluorine, chlorine, C₁-C₃-alkoxy, C₁-C₃-alkylthio, cyano, CO₂H or CO—O—C₁-C₃-alkyl, represents C₃-C₆-cycloalkyl, C₃-C₆-cycloalkylmethyl or C₅-C₆-cycloalkenyl, each of which is optionally mono- to trisubstituted by fluorine, chlorine, C₁-C₃-alkyl, C₁-C₃-haloalkyl, C₁-C₃-alkoxy, CN, CO₂H or CO—O—C₁-C₃-alkyl and each of which may optionally be interrupted by an oxygen or sulphur atom,
represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, C₁-C₄-alkyl, C₁-C₄-haloalkyl, C₁-C₄-alkoxy, C₁-C₄-haloalkoxy, nitro or cyano;
R⁷ represents hydrogen, represents C₁-C₈-alkyl, C₃-C₈-alkenyl or C₃-C₈-alkynyl, each of which is optionally mono- to pentasubstituted by fluorine, chlorine, C₁-C₃-alkoxy, C₁-C₃-alkylthio, cyano, CO₂H or CO—O—C₁-C₃-alkyl, represents C₃-C₆-cycloalkyl, C₃-C₆-cycloalkylmethyl or C₅-C₆-cycloalkenyl, each of which is optionally mono- to trisubstituted by fluorine, chlorine, C₁-C₄-alkyl, C₁-C₂-haloalkyl, C₁-C₂-alkoxy, CN, CO₂H or CO—O—C₁-C₂-alkyl and each of which may optionally be interrupted by an oxygen or sulphur atom,
represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, C₁-C₃-alkyl, C₁-C₃-haloalkyl, C₁-C₃-alkoxy, C₁-C₃-haloalkoxy, nitro or cyano;
R⁸ represents hydrogen, C₁-C₄-alkyl, C₃-C₄-alkenyl, C₃-C₄-alkynyl or C₁-C₄-alkoxy, each of which is optionally mono- to trisubstituted by fluorine and/or chlorine;
or
R⁴ and R⁷ together with the N—CO or N—S(O)₁₋₂ group to which they are attached form a 4- to 6-membered cycle which may contain one or more heteroatoms from the group consisting of sulphur, oxygen and nitrogen, where oxygen atoms must not be adjacent to one another;
or
R⁷ and R⁸ together with the nitrogen atom to which they are attached represent a saturated or unsaturated 5- or 6-membered ring which is optionally mono- or disubstituted by C₁-C₂-alkyl and which may optionally be interrupted by oxygen or sulphur;
R⁹ and R¹⁰ independently of one another represent C₁-C₆-alkyl, C₃-C₆-alkenyl, C₁-C₆-alkoxy, C₃-C₆-alkenyloxy, C₃-C₆-alkynyloxy, C₁-C₄-alkylthio, C₃-C₄-alkenylthio, each of which is optionally mono- to trisubstituted by fluorine and/or chlorine, C₃-C₆-cycloalkoxy, C₄-C₆-cycloalkenyloxy or C₃-C₆-cycloalkyl-C₁-C₂-alkyloxy, each of which is optionally mono- or disubstituted by fluorine, chlorine, C₁-C₂-alkyl, C₁-C₂ haloalkyl, C₁-C₂-alkoxy, represent phenoxy, phenylthio, benzyloxy, benzylthio, pyridinyloxy, pyrazolyloxy, pyridinylthio, pyrimidylthio, thiazolylthio, pyridylmethyloxy, pyrimidylmethyloxy, thiazolylmethyloxy, pyridylmethylthio, thiazolylmethylthio or pyrimidylmethylthio, each of which is optionally mono- or disubstituted by fluorine, chlorine, bromine, C₁-C₄-alkyl-C₁-C₂-haloalkyl, C₁-C₄-alkoxy, C₁-C₂-haloalkoxy, C₁-C₄ alkylthio, cyano or nitro;
or
R⁹ and R¹⁰ together with the phosphorus atom to which they are attached represent a five- or six-membered cycle which is optionally mono- or disubstituted by fluorine, chlorine, C₁-C₂-alkyl, C₁-C₂-alkoxy or C₁-C₂-haloalkyl and which may be interrupted by one or two oxygen and/or sulphur atoms;
and
R^{ll} and R¹² independently of one another represent C₁-C₆-alkyl, C₃-C₆-alkenyl or C₃-C₆-alkynyl, each of which is optionally mono- to trisubstituted by fluorine or chlorine.

5. A method according to claim 1, where for A, B¹, B², B³ and B⁴ either
a) 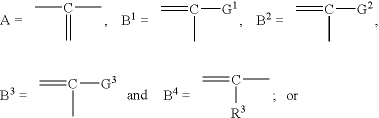
b) 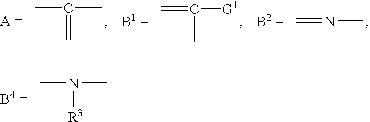
and B³=a bond; or
c) 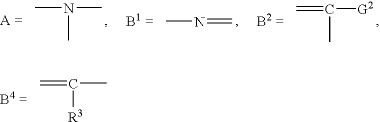
and B³=a bond;
and
X represents hydrogen, chlorine, methyl or trifluoromethyl;
Y represents (C₁-C₆)-alkyl, (C₃-₆)-alkenyl, (C₃-C₆)-alkynyl or (C₃-C₆)-cycloalkyl, where Y is unsubstituted or substituted by one to three fluorine or chlorine atoms;
or
Y represents mono- to trisubstituted phenyl having substituents from the group consisting of fluorine, chlorine, bromine, cyano, nitro, methyl, ethyl, tert-butyl, methoxy, ethoxy, trifluoromethyl, difluoromethoxy and trifluoromethoxy;
or
Y represents pyridyl which is attached in the 2- or 4-position and which may be mono- or disubstituted by identical or different substituents from the group consisting of fluorine, chlorine, bromine, cyano, methyl, ethyl, methoxy, methylthio and trifluoromethyl; or
pyrimidyl which is attached in the 4-position and which may be mono- to trisubstituted by identical or different substituents from the group consisting of fluorine, chlorine, bromine, cyano, methyl, ethyl, methoxy, methylthio and trifluoromethyl; or
thienyl which is attached in the 2- or 3-position and which may be mono- or disubstituted by identical or different substituents from the group consisting of fluorine, chlorine, bromine, cyano, nitro, methyl, ethyl, methoxy and trifluoromethyl;
Z represents chlorine, methoxy, ethoxy, methylthio, ethythio or the group 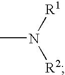
G¹, G² and G³ independently of one another represent hydrogen, chlorine, bromine, methyl, ethyl, trifluoromethyl or cyclopropyl;
L represents oxygen or sulphur;
R¹ represents hydrogen, alkyl having 1 to 6 carbon atoms which is unsubstituted or mono- to trisubstituted by identical or different substituents from the group consisting of fluorine, chlorine, methoxy, ethoxy, cyclopropyl, cyclopentyl, cyclohexyl, methylthio and ethylthio;
or
R¹ represents alkenyl having 3 to 6 carbon atoms which is unsubstituted or mono- to trisubstituted by identical or different substituents from the group consisting of fluorine, chlorine, methoxy, ethoxy, methylthio and ethylthio;
or
R¹ represents alkynyl having 3 to 6 carbon atoms which is unsubstituted or mono- to trisubstituted by identical or different substituents from the group consisting of fluorine, chlorine, methoxy, ethoxy, methylthio and ethylthio;
or
R¹ represents cycloalkyl having 3 to 6 carbon atoms which is unsubstituted or mono- to trisubstituted by identical or different substituents from the group consisting of fluorine, chlorine, methyl, ethyl, methoxy and trifluoromethyl;
R² represents hydrogen, methyl or ethyl;
or
R¹ and R² together with the nitrogen atom to which they are attached represent a saturated or unsaturated heterocyclic ring having 3 to 6 ring members, where the heterocycle optionally contains a further nitrogen, oxygen or sulphur atom as ring member and where the heterocycle may be unsubstituted or up to disubstituted by fluorine, chlorine, methyl, ethyl, trifluoromethyl, methoxy or ethoxy;
R³ represents 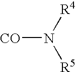
or CO—OR⁶;
R⁴ represents hydrogen, Na⁺, K⁺, ½ Ca²⁺, ½ Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺ or N(CH₃)₃—H₂—C₆H₅⁺,
represents C₁-C₆-alkyl, C₃-C₆-alkenyl or C₃-C₆-alkynyl, each of which is optionally mono- to trisubstituted by fluorine, chlorine, methoxy, ethoxy, methylthio, ethylthio, cyano, CO—O-methyl or CO—O-ethyl, represents C₃-C₆-cycloalkyl which is optionally mono- to trisubstituted by fluorine, chlorine, methyl, ethyl, trifluoromethyl, methoxy, ethoxy, cyano, CO—O-methyl or CO—O-ethyl;
R⁵ represents hydrogen, represents C₁-C₆-alkyl, C₁-C₆-alkoxy, C₃-C₆-alkenyl, C₃-C₆-alkenyloxy or C₃-C₆-alkynyl, each of which is optionally mono- to trisubstituted by fluorine and/or chlorine, or represents the groups COR⁷, S(O)₁₋₂R⁷, cyano, COOR⁷, 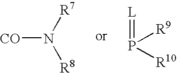
or
R⁴ and R⁵ together with the nitrogen atom to which they are attached represent a saturated 5- or 6-membered ring which may optionally be interrupted by oxygen or sulphur;
R⁶ represents hydrogen, Na⁺, K⁺, ½ Ca²⁺, ½ Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺ or N(CH₃)₃—H₂—C₆H₅⁺,
represents C₁-C₆-alkyl, C₃-C₆-alkenyl or C₃-C₆-alkynyl, each of which is optionally mono- to trisubstituted by fluorine, chlorine, methoxy, ethoxy, methylthio, ethylthio, cyano, CO₂H, CO—O-methyl or CO—O-ethyl;
R⁷ represents hydrogen, represents C₁-C₆-alkyl, C₃-C₆-alkenyl or C₃-C₆-alkynyl, each of which is optionally mono- to trisubstituted by fluorine, chlorine, methoxy, ethoxy, methylthio, ethylthio, cyano, CO—O-methyl or CO—O-ethyl, represents C₃-C₆-cycloalkyl, C₃-C₆-cycloalkylmethyl or C₅-C₆-cycloalkenyl, each of which is optionally monosubstituted by fluorine, chlorine, methyl, ethyl, methoxy, trifluoromethyl, cyano, CO₂H, CO—O-methyl or CO—O-ethyl and each of which may optionally be interrupted by an oxygen or sulphur atom, represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, methyl, trifluoromethyl, methoxy;
R⁸ represents hydrogen, methyl, ethyl, propyl, isopropyl, allyl, propargyl, C₃-C₄-alkenyl, methoxy or ethoxy;
or
R⁷ and R⁸ together with the nitrogen atom to which they are attached represent a saturated 5- or 6-membered ring which may optionally be interrupted by oxygen or sulphur;
and
R⁹ and R¹⁰ independently of one another represent methyl, ethyl, methoxy, ethoxy, propoxy, i-propoxy, butoxy, alkyloxy, methylthio, ethylthio, propylthio, butylthio, i-propylthio, sec-butylthio, allylthio, each of which is optionally mono- to trisubstituted by fluorine and/or chlorine.

6. A method according to claim 1, where for A, B¹, B², B³ and B⁴ 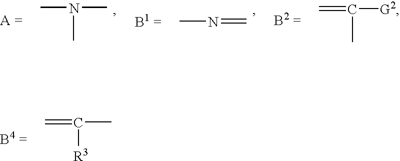
and B³=a bond;
and
X represents hydrogen, chlorine, methyl or trifluoromethyl;
Y represents (C₁-C₆)-alkyl, (C₃-₆)-alkenyl, (C₃-C₆)-alkynyl or (C₃-C₆)-cycloalkyl, where Y is unsubstituted or substituted by one to three fluorine or chlorine atoms;
or
Y represents mono- to trisubstituted phenyl having substituents from the group consisting of fluorine, chlorine, bromine, cyano, nitro, methyl, ethyl, tert-butyl, methoxy, ethoxy, trifluoromethyl, difluoromethoxy and trifluoromethoxy;
or
Y represents pyridyl which is attached in the 2- or 4-position and may be mono- or disubstituted by identical or different substituents from the group consisting of fluorine, chlorine, bromine, cyano, methyl, ethyl, methoxy, methylthio and trifluoromethyl; or
pyrimidyl which is attached in the 4-position and may be mono- to trisubstituted by identical or different substituents from the group consisting of fluorine, chlorine, bromine, cyano, methyl, ethyl, methoxy, methylthio and trifluoromethyl; or
thienyl which is attached in the 2- or 3-position and may be mono- or disubstituted by identical or different substituents from the group consisting of fluorine, chlorine, bromine, cyano, nitro, methyl, ethyl, methoxy and trifluoromethyl;
Z represents chlorine, methoxy, ethoxy, methylthio, ethythio or the group 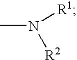
G¹, G² and G³ independently of one another represent hydrogen, chlorine, bromine, methyl, ethyl, trifluoromethyl or cyclopropyl;
L represents oxygen or sulphur;
R¹ represents hydrogen, alkyl having 1 to 6 carbon atoms which is unsubstituted or mono- to trisubstituted by identical or different substituents from the group consisting of fluorine, chlorine, methoxy, ethoxy, cyclopropyl, cyclopentyl, cyclohexyl, methylthio and ethylthio;
or
R¹ represents alkenyl having 3 to 6 carbon atoms which is unsubstituted or mono- to trisubstituted by identical or different substituents from the group consisting of fluorine, chlorine, methoxy, ethoxy, methylthio and ethylthio;
or
R¹ represents alkynyl having 3 to 6 carbon atoms which is unsubstituted or mono- to trisubstituted by identical or different substituents from the group consisting of fluorine, chlorine, methoxy, ethoxy, methylthio and ethylthio;
or
R¹ represents cycloalkyl having 3 to 6 carbon atoms which is unsubstituted or mono- to trisubstituted by identical or different substituents from the group consisting of fluorine, chlorine, methyl, ethyl, methoxy and trifluoromethyl;
R² represents hydrogen, methyl or ethyl;
or
R¹ and R² together with the nitrogen atom to which they are attached represent a saturated or unsaturated heterocyclic ring having 3 to 6 ring members, where the heterocycle optionally contains a further nitrogen, oxygen or sulphur atom as ring member and where the heterocycle may be unsubstituted or up to disubstituted by fluorine, chlorine, methyl, ethyl, trifluoromethyl, methoxy or ethoxy;
R³ represents 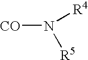
or CO—OR⁶;
R⁴ represents hydrogen,
represents C₁-C₆-alkyl, C₃-C₆-alkenyl or C₃-C₆-alkynyl, each of which is optionally mono- to trisubstituted by fluorine, chlorine, methoxy, ethoxy, methylthio, ethylthio, cyano, CO—O-methyl or CO—O-ethyl, represents C₃-C₆-cycloalkyl which is optionally mono- to trisubstituted by fluorine, chlorine, methyl, ethyl, trifluoromethyl, methoxy, ethoxy, cyano, CO—O-methyl or CO—O-ethyl;
R⁵ represents hydrogen, represents C₁-C₆-alkyl, C₁-C₆-alkoxy, C₃-C₆-alkenyl, C₃-C₆-alkenyloxy or C₃-C₆-alkynyl, each of which is optionally mono- to trisubstituted by fluorine and/or chlorine, or represents the groups COR⁷, S(O)₁₋₂R⁷, cyano, COOR⁷, 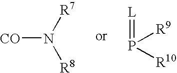
or
R⁴ and R⁵ together with the nitrogen atom to which they are attached represent a saturated 5- or 6-membered ring which may optionally be interrupted by oxygen or sulphur;
R⁶ represents hydrogen,
represents C₁-C₆-alkyl, C₃-C₆-alkenyl or C₃-C₆-alkynyl, each of which is optionally mono- to trisubstituted by fluorine, chlorine, methoxy, ethoxy, methylthio, ethylthio, cyano, CO₂H, CO—O-methyl or CO—O-ethyl;
R⁷ represents hydrogen, represents C₁-C₆-alkyl, C₃-C₆-alkenyl or C₃-C₆-alkynyl, each of which is optionally mono- to trisubstituted by fluorine, chlorine, methoxy, ethoxy, methylthio, ethylthio, cyano, CO—O-methyl or CO—O-ethyl, represents C₃-C₆-cycloalkyl, C₃-C₆-cycloalkylmethyl or C₅-C₆-cycloalkenyl, each of which is optionally monosubstituted by fluorine, chlorine, methyl, ethyl, methoxy, trifluoromethyl, cyano, CO₂H, CO—O-methyl or CO—O-ethyl and each of which may optionally be interrupted by an oxygen or sulphur atom, represents phenyl, pyridyl, thiazolyl, pyrimidyl, benzyl, phenethyl, pyridinylmethyl or thiazolylmethyl, each of which is optionally mono- to disubstituted by fluorine, chlorine, bromine, methyl, trifluoromethyl, methoxy;
R⁸ represents hydrogen, methyl, ethyl, propyl, isopropyl, allyl, propargyl, C₃-C₄-alkenyl, methoxy or ethoxy;
or
R⁷ and R⁸ together with the nitrogen atom to which they are attached represent a saturated 5- or 6-membered ring which may optionally be interrupted by oxygen or sulphur;
and
R⁹ and R¹⁰ independently of one another represent methyl, ethyl, methoxy, ethoxy, propoxy, i-propoxy, butoxy, alkyloxy, methylthio, ethylthio, propylthio, butylthio, i-propylthio, sec-butylthio, allylthio, each of which is optionally mono- to trisubstituted by fluorine and/or chlorine.

7. A mixture for controlling animal pests, said mixture comprising at least one heterocyclic carboxylic acid derivative of the formula (I) 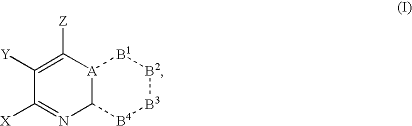
in which
A represents a nitrogen atom, in which case the bond in the pyrimidine ring is a single bond, or represents a carbon atom, in which case the bond in the pyridine ring is a double bond,
B¹ represents a nitrogen atom —N═ or represents the fragment 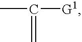
B² represents a nitrogen atom —N═ or represents the fragment 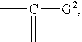
B³ represents a nitrogen atom —N═ or represents the fragment 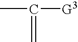
or represents a bond,
and
B⁴ represents the fragment 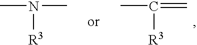
where the dashed bonds may be single bonds, double bonds or aromatic bonds;
X represents hydrogen, halogen, cyano, hydroxyl, alkyl, alkoxy, phenyl, alkylthio, alkylsulphinyl or alkylsulphonyl;
Y represents aryl, heterocyclyl, alkyl, alkenyl, alkynyl, cycloalkyl, cycloalkenyl, arylalkyl, halogen, an amino group, (C₁-C₈)-alkoxy, (C₁-C₈)-alkylthio, (C₆-C₁₀)-aryloxy, (C₆-C₁₀)-arylthio, heterocyclyloxy, (C₆-C₁₀)-aryl-(C₁-C₄)-alkoxy, (C₆-C₁₀)-aryl-(C₁-C₄)-alkylthio, heterocyclyl-(C₁-C₄)-alkoxy, heterocyclyl-(C₁-C₄)-alkylthio, C(S)OR⁸, C(O)SR⁸ or C(S)SR⁸;
Z represents hydroxyl, halogen or alkoxy, alkylthio, alkylsulphonyl or the group 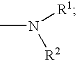
G¹, G² and G³ independently of one another represent hydrogen, halogen, alkyl or cycloalkyl, O—(C₁-C₄)-alkyl or S(O)₀₋₂(C₁-C₄)-alkyl;
L represents oxygen or sulphur;
R¹ represents hydrogen, alkyl, alkenyl, alkynyl, cycloalkyl or heterocyclyl, hydroxyl, alkoxy, amine, alkylamine or dialkylamine;
R² represents hydrogen or alkyl;
or
R¹ and R² together with the nitrogen atom to which they are attached represent a heterocyclic ring;
R³ represents 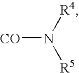
CO—OR⁶, CO—SR⁶, CS—OR⁶ or CS—SR⁶;
R⁴ represents hydrogen, alkyl, alkenyl or alkynyl, in each case saturated or unsaturated cycloalkyl which may optionally be interrupted by heteroatoms, phenyl, heteroaryl, arylalkyl or heteroarylalkyl or a cation such as, for example, a mono- or divalent metal atom or an ammonium ion;
R⁵ represents hydrogen, alkyl, alkoxy, alkenyl, alkenyloxy or alkynyl, represents the groups COR⁷, S(O)₁₋₂R⁷, cyano, COOR⁷, 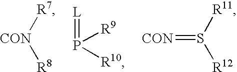
or saturated, partially or fully unsaturated or aromatic, 5- or 6-membered heterocyclyl which optionally contains one or up to three further heteroatoms selected from the group consisting of nitrogen, sulphur and oxygen atoms, where oxygen atoms must not be adjacent to one another;
or
R⁴ and R⁵ together with the nitrogen atom to which they are attached represent a saturated, unsaturated or aromatic ring which may optionally be interrupted by further heteroatoms;
R⁶ represents hydrogen, a cation, for example an ammonium ion, alkyl, alkenyl, alkynyl, cycloalkyl or cycloalkenyl which may optionally be interrupted by a heteroatom, cycloalkylalkyl or aryl, arylalkyl, heteroaryl or heteroarylalkyl;
R⁷ represents hydrogen, alkyl, alkenyl, alkynyl, represents cycloalkylalkyl, cycloalkyl or cycloalkenyl, aryl, heteroaryl, arylalkyl or heteroarylalkyl;
R⁸ represents hydrogen, alkyl, alkenyl, alkynyl, alkoxy or NH—R⁴;
or
R⁴ and R⁷ together with the N—CO or N—S(O)₁₋₂ group to which they are attached form a 4- to 8-membered cycle which may contain one or more heteroatoms from the group consisting of sulphur, oxygen and nitrogen, where oxygen atoms must not be adjacent to one another;
or
R⁷ and R⁸ together with the nitrogen atom to which they are attached represent an optionally substituted saturated or unsaturated ring which may optionally be interrupted by further heteroatoms;
R⁹ and R¹⁰ independently of one another represent alkyl, alkenyl, alkoxy, alkenyloxy, alkynyloxy, cycloalkoxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy or heteroarylalkylthio;
or
R⁹ and R¹⁰ together with the phosphorus atom to which they are attached represent an optionally substituted 5- to 7-membered cycle which may be interrupted by one or two oxygen and/or sulphur atoms;
and
R¹¹ and R¹² independently of one another represent alkyl, alkenyl, alkynyl, phenyl or phenylalkyl;
and/or at least one of the agrochemically active salts thereof and a further active compound selected from the group consisting of insecticides, attractants, sterilants, bactericides, acaricides, nematicides, fungicides, growth regulators, herbicides, safeners, fertilizers and semiochemicals.

8. A method according to claim 1, where the animal pests are selected from the group consisting of insects, parasites of the subclass of the Acari (Acarina), mites, spider mites, ticks, and nematodes.

9. A method according to claim 1 wherein said animal pests being treated are in or on plants or in or on seed of plants.

10. A method according to claim 9, where the plants are selected from the group consisting of transgenic plants.

11. Composition for controlling animal pests in and/or on plants or in and/or on seed of plants, where the composition comprises at least one heterocyclic carboxylic acid derivative of the formula (I) 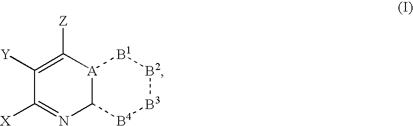
in which
A represents a nitrogen atom, in which case the bond in the pyrimidine ring is a single bond, or represents a carbon atom, in which case the bond in the pyridine ring is a double bond,
B¹ represents a nitrogen atom —N═ or represents the fragment 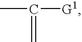
B² represents a nitrogen atom —N═ or represents the fragment 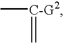
B³ represents a nitrogen atom —N═ or represents the fragment 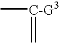
or represents a bond,
and
B⁴ represents the fragment 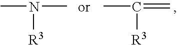
where the dashed bonds may be single bonds, double bonds or aromatic bonds;
X represents hydrogen, halogen, cyano, hydroxyl, alkyl, alkoxy, phenyl, alkylthio, alkylsulphinyl or alkylsulphonyl;
Y represents aryl, heterocyclyl, alkyl, alkenyl, alkynyl, cycloalkyl, cycloalkenyl, arylalkyl, halogen, an amino group, (C₁-C₈)-alkoxy, (C₁-C₈)-alkylthio, (C₆-C₁₀)-aryloxy, (C₆-C₁₀)-arylthio, heterocyclyloxy, (C₆-C₁₀)-aryl-(C₁-C₄)-alkoxy, (C₆-C₁₀)-aryl-(C₁-C₄)-alkylthio, heterocyclyl-(C₁-C₄)-alkoxy, heterocyclyl-(C₁-C₄)-alkylthio, C(S)OR⁸, C(O)SR⁸ or C(S)SR⁸;
Z represents hydroxyl, halogen or alkoxy, alkylthio, alkylsulphonyl or the group 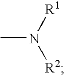
G¹, G² and G³ independently of one another represent hydrogen, halogen, alkyl or cycloalkyl, O—(C₁-C₄)-alkyl or S(O)₀₋₂(C₁-C₄)-alkyl;
L represents oxygen or sulphur;
R¹ represents hydrogen, alkyl, alkenyl, alkynyl, cycloalkyl or heterocyclyl, hydroxyl, alkoxy, amine, alkylamine or dialkylamine;
R² represents hydrogen or alkyl;
or
R¹ and R² together with the nitrogen atom to which they are attached represent a heterocyclic ring;
R³ represents 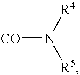
CO—OR⁶, CO—SR⁶, CS—OR⁶ or CS—SR⁶;
R⁴ represents hydrogen, alkyl, alkenyl or alkynyl, in each case saturated or unsaturated cycloalkyl which may optionally be interrupted by heteroatoms, phenyl, heteroaryl, arylalkyl or heteroarylalkyl or a cation such as, for example, a mono- or divalent metal atom or an ammonium ion;
R⁵ represents hydrogen, alkyl, alkoxy, alkenyl, alkenyloxy or alkynyl, represents the groups COR⁷, S(O)₁₋₂R⁷, cyano, COOR⁷, 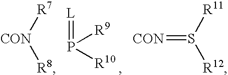
or saturated, partially or fully unsaturated or aromatic, 5- or 6-membered heterocyclyl which optionally contains one or up to three further heteroatoms selected from the group consisting of nitrogen, sulphur and oxygen atoms, where oxygen atoms must not be adjacent to one another.
or
R⁴ and R⁵ together with the nitrogen atom to which they are attached represent a saturated, unsaturated or aromatic ring which may optionally be interrupted by further heteroatoms;
R⁶ represents hydrogen, a cation, for example an ammonium ion, alkyl, alkenyl, alkynyl, cycloalkyl or cycloalkenyl which may optionally be interrupted by a heteroatom, cycloalkylalkyl or aryl, arylalkyl, heteroaryl or heteroarylalkyl;
R⁷ represents hydrogen, alkyl, alkenyl, alkynyl, represents cycloalkylalkyl, cycloalkyl or cycloalkenyl, aryl, heteroaryl, arylalkyl or heteroarylalkyl;
R⁸ represents hydrogen, alkyl, alkenyl, alkynyl, alkoxy or NH—R⁴;
or
R⁴ and R⁷ together with the N—CO or N—S(O)₁₋₂ group to which they are attached form a 4- to 8-membered cycle which may contain one or more heteroatoms from the group consisting of sulphur, oxygen and nitrogen, where oxygen atoms must not be adjacent to one another;
or
R⁷ and R⁸ together with the nitrogen atom to which they are attached represent an optionally substituted saturated or unsaturated ring which may optionally be interrupted by further heteroatoms;
R⁹ and R¹⁰ independently of one another represent alkyl, alkenyl, alkoxy, alkenyloxy, alkynyloxy, cycloalkoxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy or heteroarylalkylthio;
or
R⁹ and R¹⁰ together with the phosphorus atom to which they are attached represent an optionally substituted 5- to 7-membered cycle which may be interrupted by one or two oxygen and/or sulphur atoms;
and
R¹¹ and R¹² independently of one another represent alkyl, alkenyl, alkynyl, phenyl or phenylalkyl;
and/or at least one agrochemically active salt thereof and an agrochemically customary auxiliary and/or additive.

12. Composition according to claim 11 where the auxiliary and/or additive is selected from the group consisting of extenders and/or surfactants.

13. Composition for controlling animal pests in and/or on plants or in and/or on seed of plants, where the composition comprises at least one compound heterocyclic carboxylic acid derivative of the formula (I) 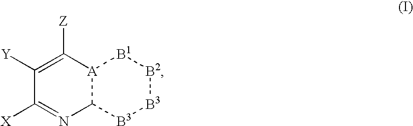
in which
A represents a nitrogen atom, in which case the bond in the pyrimidine ring is a single bond, or represents a carbon atom, in which case the bond in the pyridine ring is a double bond,
B¹ represents a nitrogen atom —N═ or represents the fragment 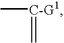
B² represents a nitrogen atom —N═ or represents the fragment 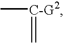
B³ represents a nitrogen atom —N═ or represents the fragment 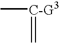
or represents a bond,
and
B⁴ represents the fragment 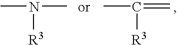
where the dashed bonds may be single bonds, double bonds or aromatic bonds;
X represents hydrogen, halogen, cyano, hydroxyl, alkyl, alkoxy, phenyl, alkylthio, alkylsulphinyl or alkylsulphonyl;
Y represents aryl, heterocyclyl, alkyl, alkenyl, alkynyl, cycloalkyl, cycloalkenyl, arylalkyl, halogen, an amino group, (C₁-C₈)-alkoxy, (C₁-C₈)-alkylthio, (C₆-C₁₀)-aryloxy, (C₆-C₁₀)-arylthio, heterocyclyloxy, (C₆-C₁₀)-aryl-(C₁-C₄)-alkoxy, (C₆-C₁₀)-aryl-(C₁-C₄)-alkylthio, heterocyclyl-(C₁-C₄)-alkoxy, heterocyclyl-(C₁-C₄)-alkylthio, C(S)OR⁸, C(O)SR⁸ or C(S)SR⁸;
Z represents hydroxyl, halogen or alkoxy, alkylthio, alkylsulphonyl or the group 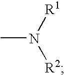
G¹, G² and G³ independently of one another represent hydrogen, halogen, alkyl or cycloalkyl, O—(C₁-C₄)-alkyl or S(O)₀₋₂(C₁-C₄)-alkyl;
L represents oxygen or sulphur;
R¹ represents hydrogen, alkyl, alkenyl, alkynyl, cycloalkyl or heterocyclyl, hydroxyl, alkoxy, amine, alkylamine or dialkylamine;
R² represents hydrogen or alkyl;
or
R¹ and R² together with the nitrogen atom to which they are attached represent a heterocyclic ring;
R³ represents 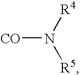
CO—OR⁶, CO—SR⁶, CS—OR⁶ or CS—SR⁶;
R⁴ represents hydrogen, alkyl, alkenyl or alkynyl, in each case saturated or unsaturated cycloalkyl which may optionally be interrupted by heteroatoms, phenyl, heteroaryl, arylalkyl or heteroarylalkyl or a cation such as, for example, a mono- or divalent metal atom or an ammonium ion;
R⁵ represents hydrogen, alkyl, alkoxy, alkenyl, alkenyloxy or alkynyl, represents the groups COR⁷, S(O)₁₋₂R⁷, cyano, COOR⁷, 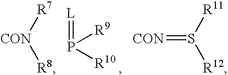
or saturated, partially or fully unsaturated or aromatic, 5- or 6-membered heterocyclyl which optionally contains one or up to three further heteroatoms selected from the group consisting of nitrogen, sulphur and oxygen atoms, where oxygen atoms must not be adjacent to one another;
or
R⁴ and R⁵ together with the nitrogen atom to which they are attached represent a saturated, unsaturated or aromatic ring which may optionally be interrupted by further heteroatoms;
R⁶ represents hydrogen, a cation, for example an ammonium ion, alkyl, alkenyl, alkynyl, cycloalkyl or cycloalkenyl which may optionally be interrupted by a heteroatom, cycloalkylalkyl or aryl, arylalkyl, heteroaryl or heteroarylalkyl;
R⁷ represents hydrogen, alkyl, alkenyl, alkynyl, represents cycloalkylalkyl, cycloalkyl or cycloalkenyl, aryl, heteroaryl, arylalkyl or heteroarylalkyl;
R⁸ represents hydrogen, alkyl, alkenyl, alkynyl, alkoxy or NH—R⁴;
or
R⁴ and R⁷ together with the N—CO or N—S(O)₁₋₂ group to which they are attached form a 4- to 8-membered cycle which may contain one or more heteroatoms from the group consisting of sulphur, oxygen and nitrogen, where oxygen atoms must not be adjacent to one another;
or
R⁷ and R⁸ together with the nitrogen atom to which they are attached represent an optionally substituted saturated or unsaturated ring which may optionally be interrupted by further heteroatoms;
R⁹ and R¹⁰ independently of one another represent alkyl, alkenyl, alkoxy, alkenyloxy, alkynyloxy, cycloalkoxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy or heteroarylalkylthio;
or
R⁹ and R¹⁰ together with the phosphorus atom to which they are attached represent an optionally substituted 5- to 7-membered cycle which may be interrupted by one or two oxygen and/or sulphur atoms;
and
R¹¹ and R¹² independently of one another represent alkyl, alkenyl, alkynyl, phenyl or phenylalkyl;
and/or at least one of its agrochemically active salts and at least one salt of the formula (II) 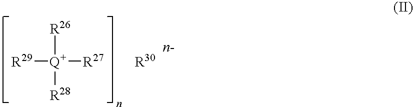
in which
Q represents nitrogen or phosphorus,
R²⁶, R²⁷, R²⁸ and R²⁹ independently of one another represent hydrogen or in each case optionally substituted C₁-C₈-alkyl or mono- or polyunsaturated, optionally substituted C₁-C₈-alkylene, where the substituents may be selected from the group consisting of halogen, nitro and cyano,
n represents 1, 2, 3 or 4,
R³⁰ represents an inorganic or organic anion.

14. Composition according to claim 13, wherein the content of said compound of formula I and/or the content of the salt of formula II is from 0.5 to 50% by weight.

15. Composition according to claim 13 wherein the content of ammonium or phosphonium salt is from 0.5 to 80 mmol/l.

16. Composition according to claim 13 wherein Q represents nitrogen.

17. Composition according to claim 16, wherein R³⁰ represents bicarbonate, tetraborate, fluoride, bromide, iodide, chloride, monohydrogenphosphate, dihydrogenphosphate, hydrogensulphate, tartrate, sulphate, nitrate, thiosulphate, thiocyanate, formate, lactate, acetate, propionate, butyrate, pentanoate, citrate or oxalate.

18. Composition according to claim 16, wherein R³⁰ represents carbonate, pentaborate, sulphite, benzoate, hydrogenoxalate, hydrogencitrate, methylsulphate or tetrafluoroborate.

19. Composition according to claim 16, wherein R³⁰ represents lactate, sulphate, nitrate, thiosulphate, thiocyanate, citrate, oxalate or formate.

20. Composition according to claim 16, wherein R³⁰ represents thiocyanate, dihydrogenphosphate, monohydrogenphosphate or sulphate.

21. Composition according to claim 13 further comprising at least one penetrant.

22. Composition according to claim 21, wherein the penetrant is a fatty alcohol alkoxylate of the formula (III)
R—O—(-AO)ᵥ₋R′ (III)
in which
R represents straight-chain or branched alkyl having 4 to 20 carbon atoms,
R′ represents hydrogen, methyl, ethyl, n-propyl, i-propyl, n-butyl, i-butyl, t-butyl, n-pentyl or n-hexyl,
AO represents an ethylene oxide radical, a propylene oxide radical, a butylene oxide radical or represents mixtures of ethylene oxide and propylene oxide radicals or butylene oxide radicals and
v represents numbers from 2 to 30,
or a mineral or vegetable oil or the ester of a mineral or vegetable oil.

23. Composition according to claim 21, wherein the penetrant is the ester of a vegetable oil.

24. Composition according to claim 21, wherein the penetrant is rapeseed oil methyl ester.

25. Composition according to claim 21 wherein the content of penetrant is from 1 to 95% by weight.

26. A Method for controlling animal pests in and/or on plants or in and/or on seed of plants, which comprises bringing the pests mentioned into direct or indirect contact with a composition as defined in claim 11.

27. Method according to claim 26, where
(i) the animal pests are selected from the group consisting of insects, parasites of the subclass of the Acari (Acarina), mites, spider mites, ticks, and nematodes;
and/or
(ii) the plants are selected from the group consisting of transgenic plants.

28. Process for preparing a composition as defined in claim 11, which comprises mixing said at least one at least one heterocyclic carboxylic acid derivative with at least one customary agrochemical auxiliary and/or additive.

29. Process according to claim 28, where the auxiliary ies and/or additive is selected from the group consisting of extenders and/or surfactants.

30. Seed treated with at least one heterocyclic carboxylic acid derivative compound as defined in claim 1.

31. Seed according to claim 30, where the seed is selected from seed of transgenic plants.
